(19) United States
(12) Patent Application Publication
(10) Pub. No.: US 2012/0207743 A1
(43) Pub. Date: Aug. 16, 2012

(54) Inhibiting Aberrant Blood Vessel Formation Using Retargeted Endopeptidases

(75) Inventors: Birgitte P.S. Jacky, Orange, CA (US); Patton E. Garay, Long Beach, CA (US); Yanira Molina, Tustin, CA (US); Joseph Francis, Languna Niguel, CA (US); Lance E. Steward, Irvine, CA (US); Sanjiv Ghanshani, Irvine, CA (US); Terrence J. Hunt, Corona, CA (US); Kei Roger Aoki, Coto de Caza, CA (US); Ester Fernandez-Salas, Fullerton, CA (US)

(73) Assignee: ALLERGAN, INC., Irvine, CA (US)

(21) Appl. No.: 13/372,018

(22) Filed: Feb. 13, 2012

Related U.S. Application Data
(60) Provisional application No. 61/442,769, filed on Feb. 14, 2011.
(60) Provisional application No. 61/445,030, filed on Feb. 21, 2011.

(51) Int. Cl. A61K 38/48; A61P 9/10; A61P 27/02; A61P 35/00; A61P 29/00; A61P 19/02; A61P 17/06; C12N 9/52; A61P 9/00

(52) U.S. Cl. 424/94.67; 435/220

(57) ABSTRACT

The present specification discloses TVEMPs, compositions comprising such TVEMPs and methods of treating a disease or disorder associated with aberrant new blood vessel formation in a mammal using such TVEMP compositions.

This application claims priority pursuant to 35 U.S.C. §119(e) to U.S. Ser. No. 61/442,769, filed Feb. 14, 2011; and U.S. Ser. No. 61/445,030, filed Feb. 21, 2011, both incorporated entirely by reference.

Mammalian cells require oxygen and nutrients for their survival and are therefore located within 100 to 200 mm of blood vessels; the diffusion limit for oxygen. For multicellular organisms to grow beyond this size, they must recruit new blood vessels by vasculogenesis and angiogenesis. In general terms, angiogenesis is the physiological process involving the growth of new blood vessels from pre-existing vessels and includes both sprouting angiogenesis and splitting angiogenesis (intussusception). In contrast, vasculogenesis is the process of blood vessel formation occurring by a de novo production of endothelial cells, i.e., the formation of new blood vessels when there are no pre-existing ones. Both angiogenesis and vasculogenesis occur in several biological processes under normal physiologic conditions. For example, vasculogenesis is critical during development as the embryo forms its circulatory and lymphatic systems. Angiogenesis is important during embryogenesis as well as in several processes in the adult, including the ovarian/menstrual cycle, growth, wound healing, and in granulation tissue.

It is believed that sprouting angiogenesis is initiated when biological signals known as angiogenic factors activate receptors present on endothelial cells of pre-existing blood vessels. In response to this signal, activated endothelial cells release proteases that degrade the basement membrane which allows endothelial cells to escape from the original (parent) vessel walls. The endothelial cells then proliferate into the stromal space surrounding matrix and form solid sprouts connecting neighboring vessels and migrate in response to an angiogenic stimulus. These sprouts then form loops to become a full-fledged vessel lumen as cells migrate to the site of angiogenesis and capillary tubes develop with formation of tight junctions and deposition of new basement membrane. Maturation of nascent vessels involves formation of a new basement membrane and investment of new vessels with pericytes and smooth muscle cells. Maintenance of new vessels depends on the survival of endothelial cells.

In splitting angiogenesis, the capillary wall extends into the lumen to split a single vessel in two. First, the two opposing capillary walls establish a zone of contact. Next, the endothelial cell junctions are reorganized and the vessel bilayer is perforated to allow growth factors and cells to penetrate into the lumen. A core is then formed between the two new vessels at the zone of contact that is filled with pericytes and myofibroblasts. These cells begin laying collagen fibers into the core to provide an extracellular matrix for growth of the vessel lumen. Finally, the core is fleshed out with no alterations to the basic structure. Intussusception is important because it is a reorganization of existing cells. It allows a vast increase in the number of capillaries without a corresponding increase in the number of endothelial cells. As such, sprouting angiogenesis is markedly different from splitting angiogenesis, however, because it forms entirely new vessels as opposed to splitting existing vessels.

Vasculogenesis occurs when endothelial precursor cells (angioblasts) migrate and differentiate in response to local cues (such as growth factors and extracellular matrix) to form new blood vessels. These vascular trees are then pruned and extended through angiogenesis. Although once thought to only occur during embryogenesis, vasculogenesis can also occur in the adult organism. Circulating endothelial progenitor cells (derivatives of stem cells) contribute to neovascularization, such as during tumor growth, retinopathies, and/or to the revascularization process following trauma, e.g., after cardiac ischemia.

Angiogenesis is determined by the balance between angiogenic and angiostatic signals. Angiogenic signals stimulate endothelial proliferation, migration and assembly into vessels and exert there effects primarily through endothelial-specific cell signaling systems including, e.g., vascular endothelial growth factor (VEGF), ephrin (Eph), fibroblast growth factor (FGF), platelet-derived growth factor (PDGF), transforming growth factor-α (TGF-α), transforming growth factor-13 (TGF-13), tumor necrosis factor-α (TNF-α), interleukin (IL), and other chemokines. On the other hand, angiogenesis is negatively regulated by the signals evoked by angiostatic factors such as thrombospondin, angiostatin, endostatin and vasohibin. Angiopoietin (Ang) 1/Tie2 signal is known to regulate both vascular quiescence and angiogenesis. The binding of these angiogenic factors to receptors in endothelial cells leads to a cascade of different signaling pathways resulting in the up- or down-regulation of genes involved in regulating the proliferation and migration of endothelial cells and promoting their survival and vascular permeability. Angiogenesis also depends on the survival of endothelial cells and this is supported by both autocrine and paracrine interactions in which pro-survival signals are secreted by endothelial cells, pericytes, and endothelial precursors.

Dysregulated blood vessel formation contributes to the pathogenesis of many diseases including retinopathy, macula degeneration, atherosclerosis, endometriosis, idiopathic pulmonary fibrosis, chronic inflammatory/fibroproliferative disorder, rheumatoid arthritis, psoriasis, and tumor progression. Both angiogenesis and vasculogenesis are increasingly being recognized for their role in promoting the pathogenesis of these diseases. In these pathological states, there is an imbalance between endogenous angiogenic and angiostatic signals, leading to an “angiogenic switch” which results in aberrant new blood vessel formation. For example, rheumatoid arthritis is associated with the unrestrained proliferation of fibroblasts and capillary blood vessels that leads to the formation of the pannus and destruction of joint spaces. Psoriasis is a well known angiogenesis-dependent skin disorder that is characterized by marked dermal neovascularization. The pathogenesis of coronary atherosclerotic plaque formation is a complex process that demonstrates features of exaggerated injury and repair including recruitment of mononuclear cells, fibroproliferation, deposition of extracellular matrix, and aberrant angiogenesis, which lead to progressive fibrosis, calcification, and eventual luminal occlusion. Idiopathic pulmonary fibrosis (IPF) is a chronic and often fatal pulmonary fibroproliferative disorder. The pathogenesis of IPF that ultimately leads to end-stage fibrosis demonstrates features of dysregulated/abnormal repair with exaggerated neovascularization/vascular remodeling, fibroproliferation, and deposition of extracellular matrix, leading to progressive fibrosis and loss of lung function. Tumors require a vascular supply to grow and aberrant blood vessel formation is associated with tumor growth and/or metastasis to another organ. Aberrant angiogenesis associated with chronic inflammation/fibroproliferative disorders has been shown to be analogous to neovascularization of tumorigenesis of cancer.

As a pathological state develops, the deregulated endothelial cells produce a wider array of angiogenic molecules having redundant functions. As such, if only one molecule (for example, VEGF) is blocked, the pathological process can switch to another molecule (for example, FGF-1 or IL-8). The field of anti-angiogenic therapy is now facing the challenge of overcoming resistance to factor-specific-targeted therapy. One approach is to administer a cocktail of different drugs to treat pathological angiogenesis. However, compounds and methods that can target aberrant new blood vessel formation using a single therapeutic molecule would be highly desirable. The present specification provides an alternative approach to resolve this issue of therapeutic resistance based on redundancy by providing molecules that can target several angiogenic pathways simultaneously thereby eliminating both the primary and redundant signals.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea) BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc. San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

A Clostridial toxin treatment inhibits neurotransmitter release by disrupting the exocytotic process used to secret the neurotransmitter into the synaptic cleft. This disruption is ultimately accomplished by intracellular delivery of a Clostridial toxin light chain comprising an enzymatic domain where it cleaves a SNARE protein essential for the exocytotic process. There is a great desire by the pharmaceutical industry to expand the use of Clostridial toxin therapies beyond its current myo-relaxant applications to treat other ailments, such a s, e.g., various kinds of sensory nerve-based ailments like chronic pain, neurogenic inflammation and urogentital disorders, as well as non-nerve-based disorders, such as, e.g., pancreatitis and cancer. One approach that is currently being exploited to expand Clostridial toxin-based therapies involves modifying a Clostridial toxin so that the modified toxin has an altered cell targeting capability for a non-Clostridial toxin target cell. This re-targeted capability is achieved by replacing a naturally-occurring targeting domain of a Clostridial toxin with a targeting domain showing a selective binding activity for a non-Clostridial toxin receptor present in a non-Clostridial toxin target cell. Such modifications to a targeting domain result in a modified toxin that is able to selectively bind to a non-Clostridial toxin receptor (target receptor) present on a non-Clostridial toxin target cell (re-targeted). A modified Clostridial toxin with a targeting activity for a non-Clostridial toxin target cell can bind to a receptor present on the non-Clostridial toxin target cell, translocate into the cytoplasm, and exert its proteolytic effect on the SNARE complex of the non-Clostridial toxin target cell. In essence, a Clostridial toxin light chain comprising an enzymatic domain is intracellularly delivered to any desired cell by selecting the appropriate targeting domain.

The present specification discloses a class of modified Clostridial toxins retargeted to a non-Clostridial toxin receptor called Targeted Vesicular Exocytosis Modulating Proteins (TVEMPs), compositions comprising TVEMPs, and methods for treating an individual suffering from disease or disorder associated with aberrant new blood vessel formation. A TVEMP is a recombinantly produced protein that comprises a targeting domain, and a Clostridial toxin translocation domain and a Clostridial toxin enzymatic domain. The targeting domain is selected for its ability to bind to a receptor present on a target cell of interest involved in neovascularization or angiogenesis like endothelial cells, endothelial progenitor cells, tip cells, stalk cells, phalanx cells, mural cells, pericytes, or macrophages. The Clostridial toxin translocation domain and enzymatic domain serve to deliver the enzymatic domain into the cytoplasm of the target cell where it cleaves its cognate SNARE substrate. SNARE protein cleavage disrupts exocytosis, the process of cellular secretion or excretion in which substances contained in intracellular vesicles are discharged from the cell by fusion of the vesicular membrane with the outer cell membrane. This disruption prevents many fundamental processes of the cell, including, without limitation, insertion of transmembrane proteins including cell-surface receptors and signal transduction proteins; transportation of extracellular matrix proteins into the extracellular space; secretion of proteins including growth factors, angiogenic factors, neurotransmitters, hormones, and any other molecules involved in cellular communication; and expulsion of material including waste products, metabolites, and other unwanted or detrimental molecules. As such, exocytosis disruption severely affects cellular metabolism and ultimately cell viability. Thus a therapeutic molecule that reduces or inhibits exocytosis of a cell decreases the ability of a cell to proliferate, migrate, and/or survive. Based on this premise, the TVEMPs disclosed herein are designed to target disease or disorders associated with aberrant new blood vessel formation, where subsequent translocation of the enzymatic domain disrupts exocytosis by SNARE protein cleavage, thereby reducing the ability of a cell to survive or promote aberrant new blood vessel formation.

Thus, aspects of the present invention provide a TVEMP comprising a targeting domain, a Clostridial toxin translocation domain and a Clostridial toxin enzymatic domain. A targeting domain may be an apelin targeting domain, an anthrax protective antigen (APA) targeting domain, an angiogenic factor with G patch and FHA domain 1 (AGGF1) targeting domain, an angiopoietin targeting domain, an angiostatin targeting domain, an angiogenin targeting domain, an endothelin targeting domain, an endothelial cell-specific molecule (ECSM) targeting domain, an ephrin targeting domain, an erythropoietin targeting domain, a hepatocyte growth factor (HGF) targeting domain, a netrin targeting domain, a brain specific angiogenesis inhibitor (BSAI) targeting domain, a Delta-like (DII) targeting domain, a jagged targeting domain, a semaphorin (Sema) targeting domain, a thrombospondin targeting domain, a chondromodulin targeting domain, a 16 kDa prolactin fragment targeting domain, a granulocyte macrophage-colony stimulating factor (GM-CSF) targeting domain, an Interferon-α (IFN-α) targeting domain, a pigment epithelium-derived factor (PEDF) targeting domain, a troponin targeting domain, a Vasohibin (VASH) targeting domain, a Urocortin (Ucn) targeting domain, a C-motif cytokine ligand targeting domain, a C—C-motif cytokine ligand targeting domain, a C—X—C-motif cytokine ligand targeting domain, and an endothelial-monocyte activating polypeptide (EMAP) targeting domain. A Clostridial toxin translocation domain may be a Clostridium botulinum toxin (BoNT) translocation domain, a Clostridium tetani toxin (TeNT) translocation domain, a Clostridium baratii toxin (BaNT) translocation domain, or a Clostridium butyricum toxin (BuNT) translocation domain. A BoNT translocation domain may be a BoNT/A translocation domain, a BoNT/B translocation domain, a BoNT/C1 translocation domain, a BoNT/D translocation domain, a BoNT/E translocation domain, a BoNT/F translocation domain, or a BoNT/G translocation domain. A Clostridial toxin enzymatic domain may be a BoNT enzymatic domain, a TeNT enzymatic domain, a BaNT enzymatic domain, or a BuNT enzymatic domain. A BoNT enzymatic domain may be a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, or a BoNT/G enzymatic domain. A TVEMP may further comprise an exogenous protease cleavage site. An exogenous protease cleavage site may be a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

Other aspects of the present invention provide a composition comprising a TVEMP comprising a targeting domain, a Clostridial toxin translocation domain and a Clostridial toxin enzymatic domain. A composition comprising a TVEMP can be a pharmaceutical composition. Such a pharmaceutical composition can comprise, in addition to a TVEMP, a pharmaceutical carrier, a pharmaceutical component, or both.

Yet other aspects of the present invention provide a method of treating a disease or disorder associated with aberrant new blood vessel formation in a mammal, the method comprising the step of administering to the mammal in need thereof a therapeutically effective amount of a composition including a TVEMP comprising a targeting domain, a Clostridial toxin translocation domain and a Clostridial toxin enzymatic domain, wherein administration of the composition reduces a symptom associated with aberrant new blood vessel formation. Useful TVEMPs include the ones disclosed herein. The disclosed methods provide a safe, inexpensive, out patient-based treatment for the treatment of diseases or disorders caused or affected by aberrant new blood vessel formation.

Yet other aspects of the present invention provide a method of treating a disease or disorder associated with aberrant new blood vessel formation in a mammal, the method comprising the step of administering to the mammal in need thereof a therapeutically effective amount of a composition including a TVEMP comprising a targeting domain, a Clostridial toxin translocation domain, a Clostridial toxin enzymatic domain, and an exogenous protease cleavage site, wherein administration of the composition reduces a symptom of a disease or disorder associated with aberrant new blood vessel formation. Useful TVEMPs include the ones disclosed herein.

Still other aspects of the present invention provide a use of a TVEMP in the manufacturing a medicament for treating a disease or disorder associated with aberrant new blood vessel formation in a mammal in need thereof, wherein the TVEMP comprising a targeting domain, a Clostridial toxin translocation domain and a Clostridial toxin enzymatic domain. Useful TVEMPs include the ones disclosed herein.

Still other aspects of the present invention provide a use of a TVEMP in the treatment of a disease or disorder associated with aberrant new blood vessel formation in a mammal in need thereof, the use comprising the step of administering to the mammal a therapeutically effective amount of the TVEMP, wherein the TVEMP comprising a targeting domain, a Clostridial toxin translocation domain, a Clostridial toxin enzymatic domain and wherein administration of the TVEMP reduces a symptom of a disease or disorder associated with aberrant new blood vessel formation. Useful TVEMPs include the ones disclosed herein.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the HN domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a targeting domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the HCC region of the Clostridial toxin binding domain is depicted. This region comprises the 13-trefoil domain which comprises in an amino to carboxyl linear organization an α-fold, a β4/β5 hairpin turn, a β-fold, a β8/β9 hairpin turn and a y-fold.

FIG. 3 shows TVEMPs with a targeting domain located at the amino terminus. FIG. 3A depicts the single-chain polypeptide form of a TVEMP with an amino to carboxyl linear organization comprising a targeting domain, a translocation domain, a di-chain loop region comprising an exogenous protease cleavage site (P), and an enzymatic domain. Upon proteolytic cleavage with a P protease, the single-chain form of the toxin is converted to the di-chain form. FIG. 3B depicts the single polypeptide form of a TVEMP with an amino to carboxyl linear organization comprising a targeting domain, an enzymatic domain, a di-chain loop region comprising an exogenous protease cleavage site (P), and a translocation domain. Upon proteolytic cleavage with a P protease, the single-chain form of the toxin is converted to the di-chain form.

FIG. 4 shows TVEMPs with a targeting domain located between the other two domains. FIG. 4A depicts the single polypeptide form of a TVEMP with an amino to carboxyl linear organization comprising an enzymatic domain, a di-chain loop region comprising an exogenous protease cleavage site (P), a targeting domain, and a translocation domain. Upon proteolytic cleavage with a P protease, the single-chain form of the toxin is converted to the di-chain form. FIG. 4B depicts the single polypeptide form of a TVEMP with an amino to carboxyl linear organization comprising a translocation domain, a di-chain loop region comprising an exogenous protease cleavage site (P), a targeting domain, and an enzymatic domain. Upon proteolytic cleavage with a P protease, the single-chain form of the toxin is converted to the di-chain form. FIG. 4C depicts the single polypeptide form of a TVEMP with an amino to carboxyl linear organization comprising an enzymatic domain, a targeting domain, a di-chain loop region comprising an exogenous protease cleavage site (P), and a translocation domain. Upon proteolytic cleavage with a P protease, the single-chain form of the toxin is converted to the di-chain form. FIG. 4D depicts the single polypeptide form of a TVEMP with an amino to carboxyl linear organization comprising a translocation domain, a targeting domain, a di-chain loop region comprising an exogenous protease cleavage site (P), and an enzymatic domain. Upon proteolytic cleavage with a P protease, the single-chain form of the toxin is converted to the di-chain form.

FIG. 5 shows TVEMPs with a targeting domain located at the carboxyl terminus. FIG. 5A depicts the single polypeptide form of a TVEMP with an amino to carboxyl linear organization comprising an enzymatic domain, a di-chain loop region comprising an exogenous protease cleavage site (P), a translocation domain, and a targeting domain. Upon proteolytic cleavage with a P protease, the single-chain form of the toxin is converted to the di-chain form. FIG. 5B depicts the single polypeptide form of a TVEMP with an amino to carboxyl linear organization comprising a translocation domain, a di-chain loop region comprising an exogenous protease cleavage site (P), an enzymatic domain, and a targeting domain. Upon proteolytic cleavage with a P protease, the single-chain form of the toxin is converted to the di-chain form.

### DETAILED DESCRIPTION

Excessive blood vessel formation in a repairing or otherwise metabolically active tissue results in inadequate delivery of oxygen, nutrients, and other substances necessary to establish essential physiological functions to the area and promote wound healing. The formation of blood vessels within a tissue may occur by angiogenesis and/or vasculogenesis. As used herein, the term “angiogenesis” refers to a physiological process involving the growth of new blood vessels from pre-existing vessels and includes sprouting angiogenesis, the formation of new blood vessel by sprouting off existing ones, and splitting angiogenesis (intussusception), the formation of new blood vessel by splitting off existing ones. As used herein, the term “vasculogenesis” refers to a physiological process involving the de novo production of new blood-vessels by proliferating endothelial stem cells, and as such, the formation of new blood vessels when there were no pre-existing ones.

Blood vessel formation, whether angiogenesis or vasculogenesis, requires signals from growth factors and other proteins that direct and control the process, such as, e.g., fibroblast growth factors (like FGF-1 and FGF-2), vascular endothelial growth factors (like VEGF-A and VEGF-C), angiopoietins (like Ang-1 and Ang-2), ephrin (Eph), platelet derived growth factor (PDGF), tumor necrosis factor-α (TNF-α), interleukin (IL), monocyte chemotactic protein-1 (MCP-1) (also known as chemokine (C—C motif) ligand 2 (CCL-2)), transforming growth factor-α (TGF-α), transforming growth factor-βs (like TGF-β1, TGF-β2, TGF-β3, and TGF-β4), chemokines, thrombospondin, angiostatin, endostatin, vasohibin, vascular cell adhesion molecules (like VCAM-1), matrix metalloproteinases (like MMP-2 and MPP-9), integrins, cadherins, plasminogen activators, plasminogen activator inhibitors, and ephrin.

The TVEMPs and compositions comprising such TVEMPs disclosed herein, reduce or otherwise inhibit aberrant blood vessel formation mediated by angiogenesis and/or vasculogenesis by inhibiting or reducing exocytosis of a target cell participating in this pathologic process. Without wishing to be bound by any particular theory, the compounds, compositions and methods disclosed herein target cells involved in the primary and/or redundant cell signaling systems that stimulate aberrant new blood vessel formation. The TVEMPs and methods disclosed herein disrupt the ability of the target cell from participating in the multiple signaling cascades necessary for the proliferation, migration and/or survival of endothelial cells. This disruption causes the endothelial cells to become quiescent and/or die. For example, disruption of exocytosis in the target cell will stop the incorporation of receptors into its plasma membrane thereby preventing the target cell from binding and transducing signals from the pro-angiogenic molecules secreted by other cells or present in the extracellular matrix. In a similar manner, the lack of receptors that initiate the maintenance or survival cues will result in the generation of apoptotic signals involved in programmed cell death. Alternatively, disruption of exocytosis in a target cell will stop the release of pro-angiogenic molecules secreted by the target cells, thereby eliminating the stimulatory signals necessary to begin blood vessel formation. Likewise, inhibition or reduction of paracrine and/or autocrine loops by blocking exocytosis and the secretion of the pro-survival signals will eliminate the signals necessary for endothelial cell maintenance and survival. Moreover, migration of endothelial cells and recruitment of pericytes and endothelial precursors rely on migration factor gradients surrounding endothelial cells. Migratory signals can also be disrupted as the secretion of these factors will be inhibited by exocytotic disruption. As such, unlike current approaches that administer a cocktail of different drugs to resolve this issue of therapeutic resistance based on redundancy, the present specification discloses TVEMPs that provide an alternative approach by providing one molecule that simultaneously targets multiple pathways responsible for the primary and redundant signals necessary for aberrant new blood vessel formation.

Aspects of the present invention provide, in part, a TVEMP. As used herein, a “TVEMP” means any molecule comprising a targeting domain, a Clostridial toxin translocation domain and a Clostridial toxin enzymatic domain.

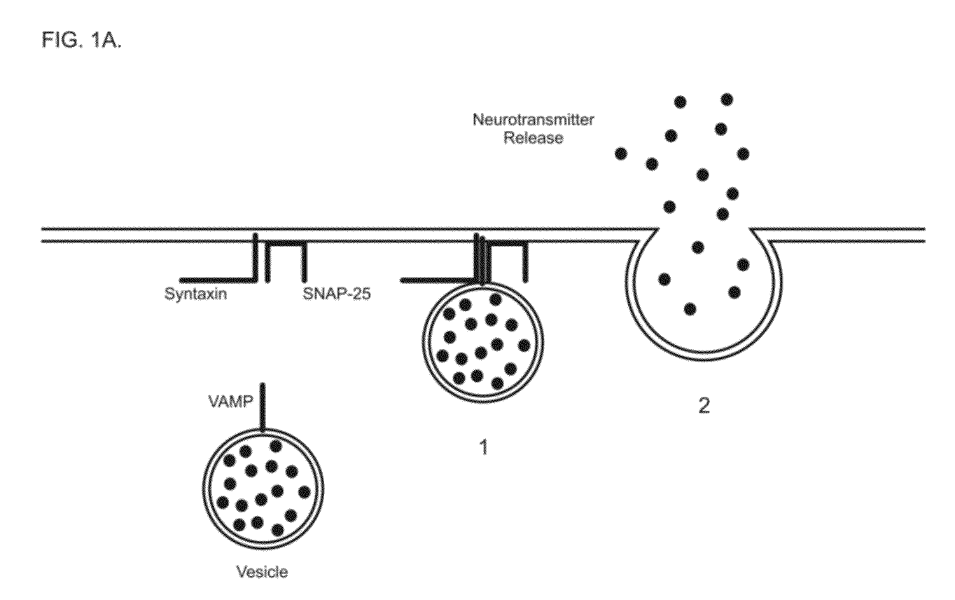

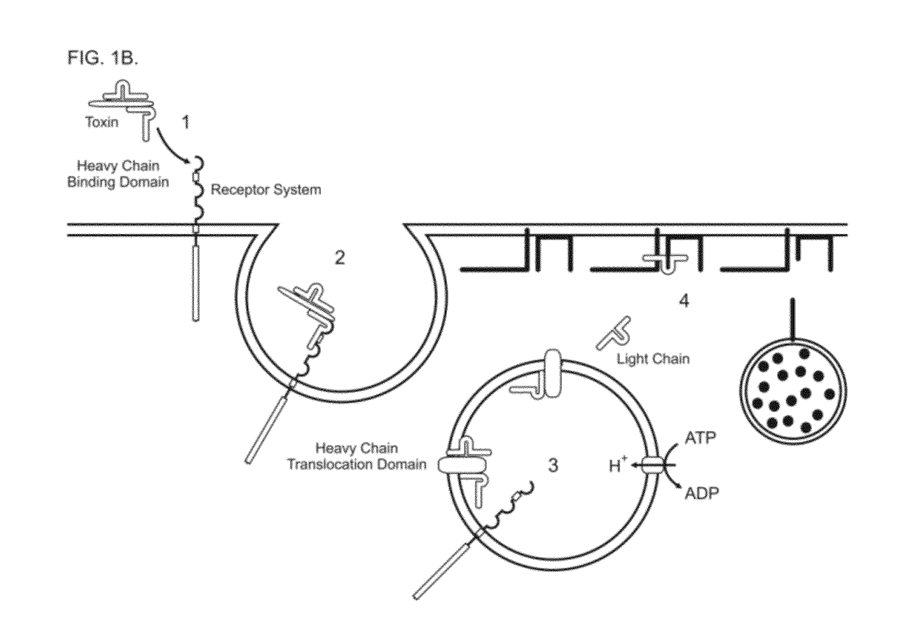

Clostridial toxins are each translated as a single chain polypeptide of approximately 150 kDa that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC(H_{N}) that facilitates release of the LC from intracellular vesicles into the cytoplasm of the target cell; and 3) a binding domain found within the carboxyl-terminal half of the HC(H_{C}) that determines the binding activity and binding specificity of the toxin to the receptor complex located at the surface of the target cell. D. B. Lacy and R. C. Stevens, Sequence Homology and Structural Analysis of the Clostridial Neurotoxins, J. Mol. Biol. 291: 1091-1104 (1999). The H_{C} domain comprises two distinct structural features of roughly equal size, separated by an α-helix, designated the H_{CN} and H_{CC} subdomains. Table 1 gives approximate boundary regions for each domain and subdomain found in exemplary Clostridial toxins.

TABLE 1
Clostridial Toxin Reference Sequences and Regions

| | SEQ ID | | Di-Chain | | H_{C} | | |
|---|---|---|---|---|---|---|---|
| Toxin | NO: | LC | Loop | H_{N} | H_{CN} | α-Linker | H_{CC} |
| BoNT/A | 1 | M1/P2-L429 | C430-C454 | I455-I873 | I874-N1080 | E1081-Q1091 | S1092-L1296 |
| BoNT/B | 6 | M1/P2-M436 | C437-C446 | I447-I860 | L861-S1067 | Q1068-Q1078 | S1079-E1291 |
| BoNT/C1 | 11 | M1/P2-F436 | C437-C453 | R454-I868 | N869-D1081 | G1082-L1092 | Q1093-E1291 |
| BoNT/D | 13 | M1/T2-V436 | C437-C450 | I451-I864 | N865-S1069 | N1069-Q1079 | I1080-E1276 |
| BoNT/E | 15 | M1/P2-F411 | C412-C426 | I427-I847 | K848-D1055 | E1056-E1066 | P1067-K1252 |
| BoNT/F | 18 | M1/P2-F428 | C429-C445 | I446-I865 | K866-D1075 | K1076-E1086 | P1087-E1274 |
| BoNT/G | 21 | M1/P2-M435 | C436-C450 | I451-I865 | S866-N1075 | A1076-Q1086 | S1087-E1297 |
| TeNT | 22 | M1/P2-L438 | C439-C467 | I468-L881 | K882-N1097 | P1098-Y1108 | L1109-D1315 |
| BaNT | 23 | M1/P2-L420 | C421-C435 | I436-I857 | I858-D1064 | K1065-E1075 | P1076-E1268 |
| BuNT | 24 | M1/P2-F411 | C412-C426 | I427-I847 | K848-D1055 | E1056-E1066 | P1067-K1251 |

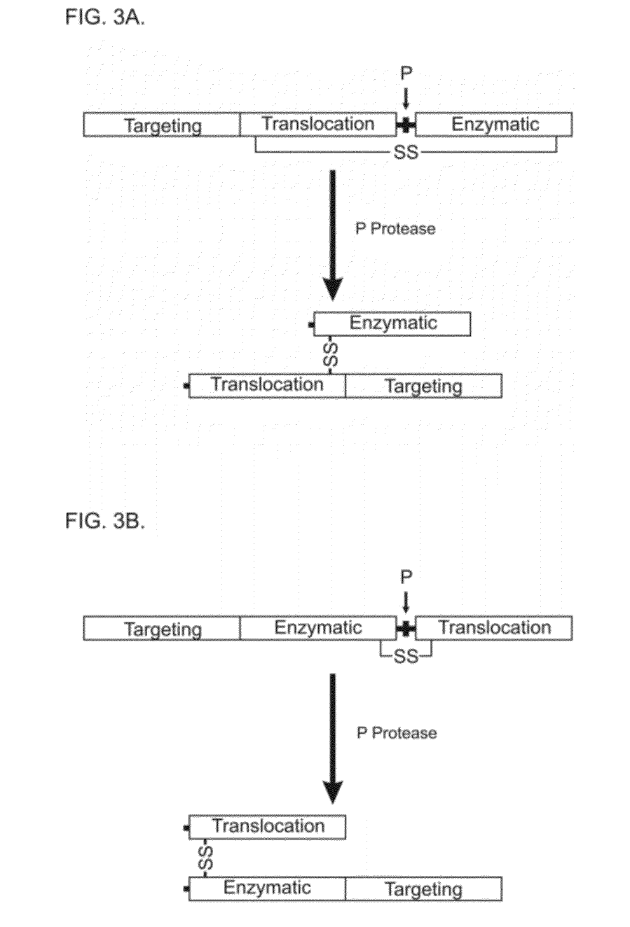

The binding, translocation, and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although the applicants have no wish to be limited by the following description, the intoxication mechanism can be described as comprising at least four steps: 1) receptor binding, 2) complex internalization, 3) light chain translocation, and 4) enzymatic target modification (FIG. 3). The process is initiated when the H_{C} domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

Aspects of the present specification provide, in part, a TVEMP comprising a Clostridial toxin enzymatic domain. As used herein, the term “Clostridial toxin enzymatic domain” refers to any Clostridial toxin polypeptide that can execute the enzymatic target modification step of the intoxication process. Thus, a Clostridial toxin enzymatic domain specifically targets a Clostridial toxin substrate and encompasses the proteolytic cleavage of a Clostridial toxin substrate, such as, e.g., SNARE proteins like a SNAP-25 substrate, a VAMP substrate, and a Syntaxin substrate. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; and non-naturally occurring Clostridial toxin enzymatic domain variants, such as, e.g., conservative Clostridial toxin enzymatic domain variants, non-conservative Clostridial toxin enzymatic domain variants, active Clostridial toxin enzymatic domain fragments thereof, or any combination thereof.

As used herein, the term “Clostridial toxin enzymatic domain variant,” whether naturally-occurring or non-naturally-occurring, refers to a Clostridial toxin enzymatic domain that has at least one amino acid change from the corresponding region of the disclosed reference sequences (Table 1) and can be described in percent identity to the corresponding region of that reference sequence. Unless expressly indicated, Clostridial toxin enzymatic domain variants useful to practice disclosed embodiments are variants that execute the enzymatic target modification step of the intoxication process. As non-limiting examples, a BoNT/A enzymatic domain variant will have at least one amino acid difference, such as, e.g., an amino acid substitution, deletion or addition, as compared to amino acids ½-429 of SEQ ID NO: 1; a BoNT/B enzymatic domain variant will have at least one amino acid difference, such as, e.g., an amino acid substitution, deletion or addition, as compared to amino acids ½-436 of SEQ ID NO: 6; a BoNT/C1 enzymatic domain variant will have at least one amino acid difference, such as, e.g., an amino acid substitution, deletion or addition, as compared to amino acids ½-436 of SEQ ID NO: 11; a BoNT/D enzymatic domain variant will have at least one amino acid difference, such as, e.g., an amino acid substitution, deletion or addition, as compared to amino acids ½-436 of SEQ ID NO: 13; a BoNT/E enzymatic domain variant will have at least one amino acid difference, such as, e.g., an amino acid substitution, deletion or addition, as compared to amino acids ½-411 of SEQ ID NO: 15; a BoNT/F enzymatic domain variant will have at least one amino acid difference, such as, e.g., an amino acid substitution, deletion or addition, as compared to amino acids ½-428 of SEQ ID NO: 18; a BoNT/G enzymatic domain variant will have at least one amino acid difference, such as, e.g., an amino acid substitution, deletion or addition, as compared to amino acids ½-438 of SEQ ID NO: 21; a TeNT enzymatic domain variant will have at least one amino acid difference, such as, e.g., an amino acid substitution, deletion or addition, as compared to amino acids ½-438 of SEQ ID NO: 22; a BaNT enzymatic domain variant will have at least one amino acid difference, such as, e.g., an amino acid substitution, deletion or addition, as compared to amino acids ½-420 of SEQ ID NO: 23; and a BuNT enzymatic domain variant will have at least one amino acid difference, such as, e.g., an amino acid substitution, deletion or addition, as compared to amino acids ½-411 of SEQ ID NO: 24.

It is recognized by those of skill in the art that within each serotype of Clostridial toxin there can be naturally occurring Clostridial toxin enzymatic domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific enzymatic domain subtypes showing about 80% to 95% amino acid identity when compared to the BoNT/A enzymatic domain of SEQ ID NO: 1. As used herein, the term “naturally occurring Clostridial toxin enzymatic domain variant” refers to any Clostridial toxin enzymatic domain produced by a naturally-occurring process, including, without limitation, Clostridial toxin enzymatic domain isoforms produced from alternatively-spliced transcripts, Clostridial toxin enzymatic domain isoforms produced by spontaneous mutation and Clostridial toxin enzymatic domain subtypes. A naturally occurring Clostridial toxin enzymatic domain variant can function in substantially the same manner as the reference Clostridial toxin enzymatic domain on which the naturally occurring Clostridial toxin enzymatic domain variant is based, and can be substituted for the reference Clostridial toxin enzymatic domain in any aspect of the present specification.

A non-limiting examples of a naturally occurring Clostridial toxin enzymatic domain variant is a Clostridial toxin enzymatic domain isoform such as, e.g., a BoNT/A enzymatic domain isoform, a BoNT/B enzymatic domain isoform, a BoNT/C1 enzymatic domain isoform, a BoNT/D enzymatic domain isoform, a BoNT/E enzymatic domain isoform, a BoNT/F enzymatic domain isoform, a BoNT/G enzymatic domain isoform, a TeNT enzymatic domain isoform, a BaNT enzymatic domain isoform, and a BuNT enzymatic domain isoform. Another non-limiting examples of a naturally occurring Clostridial toxin enzymatic domain variant is a Clostridial toxin enzymatic domain subtype such as, e.g., an enzymatic domain from subtype BoNT/A1, BoNT/A2, BoNT/A3, BoNT/A4, or BoNT/A5; an enzymatic domain from subtype BoNT/B1, BoNT/B2, BoNT/Bbv, or BoNT/Bnp; an enzymatic domain from subtype BoNT/C1-1 or BoNT/C1-2; an enzymatic domain from subtype BoNT/E1, BoNT/E2 and BoNT/E3; an enzymatic domain from subtype BoNT/F1, BoNT/F2, or BoNT/F3; and an enzymatic domain from subtype BuNT-1 or BuNT-2.

As used herein, the term “non-naturally occurring Clostridial toxin enzymatic domain variant” refers to any Clostridial toxin enzymatic domain produced with the aid of human manipulation, including, without limitation, Clostridial toxin enzymatic domains produced by genetic engineering using random mutagenesis or rational design and Clostridial toxin enzymatic domains produced by chemical synthesis. Non-limiting examples of non-naturally occurring Clostridial toxin enzymatic domain variants include, e.g., conservative Clostridial toxin enzymatic domain variants, non-conservative Clostridial toxin enzymatic domain variants, Clostridial toxin enzymatic domain chimeric variants, and active Clostridial toxin enzymatic domain fragments.

As used herein, the term “conservative Clostridial toxin enzymatic domain variant” refers to a Clostridial toxin enzymatic domain that has at least one amino acid substituted by another amino acid or an amino acid analog that has at least one property similar to that of the original amino acid from the reference Clostridial toxin enzymatic domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin enzymatic domain variant can function in substantially the same manner as the reference Clostridial toxin enzymatic domain on which the conservative Clostridial toxin enzymatic domain variant is based, and can be substituted for the reference Clostridial toxin enzymatic domain in any aspect of the present specification. Non-limiting examples of a conservative Clostridial toxin enzymatic domain variant include, e.g., conservative BoNT/A enzymatic domain variants, conservative BoNT/B enzymatic domain variants, conservative BoNT/C1 enzymatic domain variants, conservative BoNT/D enzymatic domain variants, conservative BoNT/E enzymatic domain variants, conservative BoNT/F enzymatic domain variants, conservative BoNT/G enzymatic domain variants, conservative TeNT enzymatic domain variants, conservative BaNT enzymatic domain variants, and conservative BuNT enzymatic domain variants.

As used herein, the term “non-conservative Clostridial toxin enzymatic domain variant” refers to a Clostridial toxin enzymatic domain in which 1) at least one amino acid is deleted from the reference Clostridial toxin enzymatic domain on which the non-conservative Clostridial toxin enzymatic domain variant is based; 2) at least one amino acid added to the reference Clostridial toxin enzymatic domain on which the non-conservative Clostridial toxin enzymatic domain is based; or 3) at least one amino acid is substituted by another amino acid or an amino acid analog that does not share any property similar to that of the original amino acid from the reference Clostridial toxin enzymatic domain sequence (Table 1). A non-conservative Clostridial toxin enzymatic domain variant can function in substantially the same manner as the reference Clostridial toxin enzymatic domain on which the non-conservative Clostridial toxin enzymatic domain variant is based, and can be substituted for the reference Clostridial toxin enzymatic domain in any aspect of the present specification. Non-limiting examples of a non-conservative Clostridial toxin enzymatic domain variant include, e.g., non-conservative BoNT/A enzymatic domain variants, non-conservative BoNT/B enzymatic domain variants, non-conservative BoNT/C1 enzymatic domain variants, non-conservative BoNT/D enzymatic domain variants, non-conservative BoNT/E enzymatic domain variants, non-conservative BoNT/F enzymatic domain variants, non-conservative BoNT/G enzymatic domain variants, and non-conservative TeNT enzymatic domain variants, non-conservative BaNT enzymatic domain variants, and non-conservative BuNT enzymatic domain variants.

As used herein, the term “active Clostridial toxin enzymatic domain fragment” refers to any of a variety of Clostridial toxin fragments comprising the enzymatic domain can be useful in aspects of the present specification with the proviso that these enzymatic domain fragments can specifically target the core components of the neurotransmitter release apparatus and thus participate in executing the overall cellular mechanism whereby a Clostridial toxin proteolytically cleaves a substrate. The enzymatic domains of Clostridial toxins are approximately 420-460 amino acids in length and comprise an enzymatic domain (Table 1). Research has shown that the entire length of a Clostridial toxin enzymatic domain is not necessary for the enzymatic activity of the enzymatic domain. As a non-limiting example, the first eight amino acids of the BoNT/A enzymatic domain are not required for enzymatic activity. As another non-limiting example, the first eight amino acids of the TeNT enzymatic domain are not required for enzymatic activity. Likewise, the carboxyl-terminus of the enzymatic domain is not necessary for activity. As a non-limiting example, the last 32 amino acids of the BoNT/A enzymatic domain are not required for enzymatic activity. As another non-limiting example, the last 31 amino acids of the TeNT enzymatic domain are not required for enzymatic activity. Thus, aspects of this embodiment include Clostridial toxin enzymatic domains comprising an enzymatic domain having a length of, e.g., at least 350, 375, 400, 425, or 450 amino acids. Other aspects of this embodiment include Clostridial toxin enzymatic domains comprising an enzymatic domain having a length of, e.g., at most 350, 375, 400, 425, or 450 amino acids.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cedric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

The present specification describes various polypeptide variants where one amino acid is substituted for another, such as, e.g., Clostridial toxin enzymatic domain variants, Clostridial toxin translocation domain variants, targeting domain variants, and protease cleavage site variants, A substitution can be assessed by a variety of factors, such as, e.g., the physic properties of the amino acid being substituted (Table 2) or how the original amino acid would tolerate a substitution (Table 3). The selections of which amino acid can be substituted for another amino acid in a polypeptide are known to a person of ordinary skill in the art.

TABLE 2
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 3
Amino Acid Substitutions

| Amino | Favored | | |
|---|---|---|---|
| Acid | Substitution | Neutral Substitutions | Disfavored substitution |
| A | G, S, T | C, E, I, K, M, L, P, Q, R, V | D, F, H, N, Y, W |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, Y |
| F | M, L, W, Y | C, I, V | A, D, E, G, H, K, N, P, Q, R, S, T |
| G | A, S | D, K, N, P, Q, R | C, E, F, H, I, L, M, T, V, W, Y |
| H | N, Y | C, D, E, K, Q, R, S, T, W | A, F, G, I, L, M, P, V |
| I | V, L, M | A, C, T, F, Y | D, E, G, H, K, N, P, Q, R, S, W |
| K | Q, E, R | A, D, G, H, M, N, P, S, T | C, F, I, L, V, W, Y |
| L | F, I, M, V | A, C, W, Y | D, E, G, H, K, N, P, Q, R, S, T |
| M | F, I, L, V | A, C, R, Q, K, T, W, Y | D, E, G, H, N, P, S |
| N | D, H, S | E, G, K, Q, R, T | A, C, F, I, L, M, P, V, W, Y |
| P | — | A, D, E, G, K, Q, R, S, T | C, F, H, I, L, M, N, V, W, Y |
| Q | E, K, R | A, D, G, H, M, N, P, S, T | C, F, I, L, V, W, Y |
| R | K, Q | A, D, E, G, H, M, N, P, S, T | C, F, I, L, V, W, Y |
| S | A, N, T | C, D, E, G, H, K, P, Q, R, T | F, I, L, M, V, W, Y |
| T | S | A, C, D, E, H, I, K, M, N, P, | F, G, L, W, Y |
| | | Q, R, V | |
| V | I, L, M | A, C, F, T, Y | D, E, G, H, K, N, P, Q, R, S, W |
| W | F, Y | H, L, M | A, C, D, E, G, I, K, N, P, Q, R, S, |
| | | | T, V |
| Y | F, H, W | C, I, L, M, V | A, D, E, G, K, N, P, Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

Thus, in an embodiment, a TVEMP disclosed herein comprises a Clostridial toxin enzymatic domain. In an aspect of this embodiment, a Clostridial toxin enzymatic domain comprises a naturally occurring Clostridial toxin enzymatic domain variant, such as, e.g., a Clostridial toxin enzymatic domain isoform or a Clostridial toxin enzymatic domain subtype. In another aspect of this embodiment, a Clostridial toxin enzymatic domain comprises a non-naturally occurring Clostridial toxin enzymatic domain variant, such as, e.g., a conservative Clostridial toxin enzymatic domain variant, a non-conservative Clostridial toxin enzymatic domain variant, an active Clostridial toxin enzymatic domain fragment, or any combination thereof.

In another embodiment, a hydrophic amino acid at one particular position in the polypeptide chain of the Clostridial toxin enzymatic domain can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin enzymatic domain can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin enzymatic domain can be substituted with another aromatic amino acid. Examples of aromatic amino acids include, e.g., F, H, W and Y. In still another aspect of this embodiment, a stacking amino acid at one particular position in the polypeptide chain of the Clostridial toxin enzymatic domain can be substituted with another stacking amino acid. Examples of stacking amino acids include, e.g., F, H, W and Y. In a further aspect of this embodiment, a polar amino acid at one particular position in the polypeptide chain of the Clostridial toxin enzymatic domain can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin enzymatic domain can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin enzymatic domain can be substituted with another positive charged amino acid. Examples of positive charged amino acids include, e.g., K, R, and H. In a still further aspect of this embodiment, a negative charged amino acid at one particular position in the polypeptide chain of the Clostridial toxin enzymatic domain can be substituted with another negative charged amino acid. Examples of negative charged amino acids include, e.g., D and E. In another aspect of this embodiment, a small amino acid at one particular position in the polypeptide chain of the Clostridial toxin enzymatic domain can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin enzymatic domain can be substituted with another C-beta branching amino acid. Examples of C-beta branching amino acids include, e.g., I, T and V.

In another embodiment, a Clostridial toxin enzymatic domain comprises a BoNT/A enzymatic domain. In an aspect of this embodiment, a BoNT/A enzymatic domain comprises the enzymatic domains of SEQ ID NO: 1, SEQ ID NO: 2, SEQ ID NO: 3, SEQ ID NO: 4, or SEQ ID NO: 5. In other aspects of this embodiment, a BoNT/A enzymatic domain comprises amino acids ½-429 of SEQ ID NO: 1. In another aspect of this embodiment, a BoNT/A enzymatic domain comprises a naturally occurring BoNT/A enzymatic domain variant, such as, e.g., an enzymatic domain from a BoNT/A isoform or an enzymatic domain from a BoNT/A subtype. In another aspect of this embodiment, a BoNT/A enzymatic domain comprises a naturally occurring BoNT/A enzymatic domain variant of SEQ ID NO: 1, SEQ ID NO: 2, SEQ ID NO: 3, SEQ ID NO: 4, or SEQ ID NO: 5, such as, e.g., a BoNT/A isoform enzymatic domain or a BoNT/A subtype enzymatic domain. In another aspect of this embodiment, a BoNT/A enzymatic domain comprises amino acids ½-429 of a naturally occurring BoNT/A enzymatic domain variant of SEQ ID NO: 1, such as, e.g., a BoNT/A isoform enzymatic domain or a BoNT/A subtype enzymatic domain. In still another aspect of this embodiment, a BoNT/A enzymatic domain comprises a non-naturally occurring BoNT/A enzymatic domain variant, such as, e.g., a conservative BoNT/A enzymatic domain variant, a non-conservative BoNT/A enzymatic domain variant, an active BoNT/A enzymatic domain fragment, or any combination thereof. In still another aspect of this embodiment, a BoNT/A enzymatic domain comprises the enzymatic domain of a non-naturally occurring BoNT/A enzymatic domain variant of SEQ ID NO: 1, SEQ ID NO: 2, SEQ ID NO: 3, SEQ ID NO: 4, or SEQ ID NO: 5, such as, e.g., a conservative BoNT/A enzymatic domain variant, a non-conservative BoNT/A enzymatic domain variant, an active BoNT/A enzymatic domain fragment, or any combination thereof. In still another aspect of this embodiment, a BoNT/A enzymatic domain comprises amino acids ½-429 of a non-naturally occurring BoNT/A enzymatic domain variant of SEQ ID NO: 1, such as, e.g., a conservative BoNT/A enzymatic domain variant, a non-conservative BoNT/A enzymatic domain variant, an active BoNT/A enzymatic domain fragment, or any combination thereof.

In other aspects of this embodiment, a BoNT/A enzymatic domain comprises a polypeptide having an amino acid identity of, e.g., at least 70%, at least 75%, at least 80%, at least 85%, at least 90%, or at least 95% to the enzymatic domain of SEQ ID NO: 1, SEQ ID NO: 2, SEQ ID NO: 3, SEQ ID NO: 4, or SEQ ID NO: 5; or at most 70%, at most 75%, at most 80%, at most 85%, at most 90%, or at most 95% to the enzymatic domain of SEQ ID NO: 1, SEQ ID NO: 2, SEQ ID NO: 3, SEQ ID NO: 4, or SEQ ID NO: 5. In yet other aspects of this embodiment, a BoNT/A enzymatic domain comprises a polypeptide having an amino acid identity of, e.g., at least 70%, at least 75%, at least 80%, at least 85%, at least 90%, or at least 95% to amino acids ½-429 of SEQ ID NO: 1; or at most 70%, at most 75%, at most 80%, at most 85%, at most 90%, or at most 95% to amino acids ½-429 of SEQ ID NO: 1.

In other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5. In yet other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-429 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids ½-429 of SEQ ID NO: 1. In still other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5. In further other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-429 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids ½-429 of SEQ ID NO: 1.

In another embodiment, a Clostridial toxin enzymatic domain comprises a BoNT/B enzymatic domain. In an aspect of this embodiment, a BoNT/B enzymatic domain comprises the enzymatic domains of SEQ ID NO: 6, SEQ ID NO: 7, SEQ ID NO: 8, SEQ ID NO: 9, or SEQ ID NO: 10. In other aspects of this embodiment, a BoNT/B enzymatic domain comprises amino acids ½-436 of SEQ ID NO: 6. In another aspect of this embodiment, a BoNT/B enzymatic domain comprises a naturally occurring BoNT/B enzymatic domain variant, such as, e.g., an enzymatic domain from a BoNT/B isoform or an enzymatic domain from a BoNT/B subtype. In another aspect of this embodiment, a BoNT/B enzymatic domain comprises a naturally occurring BoNT/B enzymatic domain variant of SEQ ID NO: 6, SEQ ID NO: 7, SEQ ID NO: 8, SEQ ID NO: 9, or SEQ ID NO: 10, such as, e.g., a BoNT/B isoform enzymatic domain or a BoNT/B subtype enzymatic domain. In another aspect of this embodiment, a BoNT/B enzymatic domain comprises amino acids ½-436 of a naturally occurring BoNT/B enzymatic domain variant of SEQ ID NO: 6, such as, e.g., a BoNT/B isoform enzymatic domain or a BoNT/B subtype enzymatic domain. In still another aspect of this embodiment, a BoNT/B enzymatic domain comprises a non-naturally occurring BoNT/B enzymatic domain variant, such as, e.g., a conservative BoNT/B enzymatic domain variant, a non-conservative BoNT/B enzymatic domain variant, an active BoNT/B enzymatic domain fragment, or any combination thereof. In still another aspect of this embodiment, a BoNT/B enzymatic domain comprises the enzymatic domain of a non-naturally occurring BoNT/B enzymatic domain variant of SEQ ID NO: 6, SEQ ID NO: 7, SEQ ID NO: 8, SEQ ID NO: 9, or SEQ ID NO: 10, such as, e.g., a conservative BoNT/B enzymatic domain variant, a non-conservative BoNT/B enzymatic domain variant, an active BoNT/B enzymatic domain fragment, or any combination thereof. In still another aspect of this embodiment, a BoNT/B enzymatic domain comprises amino acids ½-436 of a non-naturally occurring BoNT/B enzymatic domain variant of SEQ ID NO: 6, such as, e.g., a conservative BoNT/B enzymatic domain variant, a non-conservative BoNT/B enzymatic domain variant, an active BoNT/B enzymatic domain fragment, or any combination thereof.

In other aspects of this embodiment, a BoNT/B enzymatic domain comprises a polypeptide having an amino acid identity of, e.g., at least 70%, at least 75%, at least 80%, at least 85%, at least 90%, or at least 95% to the enzymatic domain of SEQ ID NO: 6, SEQ ID NO: 7, SEQ ID NO: 8, SEQ ID NO: 9, or SEQ ID NO: 10; or at most 70%, at most 75%, at most 80%, at most 85%, at most 90%, or at most 95% to the enzymatic domain of SEQ ID NO: 6, SEQ ID NO: 7, SEQ ID NO: 8, SEQ ID NO: 9, or SEQ ID NO: 10. In yet other aspects of this embodiment, a BoNT/B enzymatic domain comprises a polypeptide having an amino acid identity of, e.g., at least 70%, at least 75%, at least 80%, at least 85%, at least 90%, or at least 95% to amino acids ½-436 of SEQ ID NO: 6; or at most 70%, at most 75%, at most 80%, at most 85%, at most 90%, or at most 95% to amino acids ½-436 of SEQ ID NO: 6.

In other aspects of this embodiment, a BoNT/B enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10. In yet other aspects of this embodiment, a BoNT/B enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 6. In still other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10. In further other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 6; or at most 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 6.

In another embodiment, a Clostridial toxin enzymatic domain comprises a BoNT/C1 enzymatic domain. In an aspect of this embodiment, a BoNT/C1 enzymatic domain comprises the enzymatic domains of SEQ ID NO: 11 or SEQ ID NO: 12. In other aspects of this embodiment, a BoNT/C1 enzymatic domain comprises amino acids ½-436 of SEQ ID NO: 11. In another aspect of this embodiment, a BoNT/C1 enzymatic domain comprises a naturally occurring BoNT/C1 enzymatic domain variant, such as, e.g., an enzymatic domain from a BoNT/C1 isoform or an enzymatic domain from a BoNT/C1 subtype. In another aspect of this embodiment, a BoNT/C1 enzymatic domain comprises a naturally occurring BoNT/C1 enzymatic domain variant of SEQ ID NO: 11 or SEQ ID NO: 12, such as, e.g., a BoNT/C1 isoform enzymatic domain or a BoNT/C1 subtype enzymatic domain. In another aspect of this embodiment, a BoNT/C1 enzymatic domain comprises amino acids ½-436 of a naturally occurring BoNT/C1 enzymatic domain variant of SEQ ID NO: 11, such as, e.g., a BoNT/C1 isoform enzymatic domain or a BoNT/C1 subtype enzymatic domain. In still another aspect of this embodiment, a BoNT/C1 enzymatic domain comprises a non-naturally occurring BoNT/C1 enzymatic domain variant, such as, e.g., a conservative BoNT/C1 enzymatic domain variant, a non-conservative BoNT/C1 enzymatic domain variant, an active BoNT/C1 enzymatic domain fragment, or any combination thereof. In still another aspect of this embodiment, a BoNT/C1 enzymatic domain comprises the enzymatic domain of a non-naturally occurring BoNT/C1 enzymatic domain variant of SEQ ID NO: 11 or SEQ ID NO: 12, such as, e.g., a conservative BoNT/C1 enzymatic domain variant, a non-conservative BoNT/C1 enzymatic domain variant, an active BoNT/C1 enzymatic domain fragment, or any combination thereof. In still another aspect of this embodiment, a BoNT/C1 enzymatic domain comprises amino acids ½-436 of a non-naturally occurring BoNT/C1 enzymatic domain variant of SEQ ID NO: 11, such as, e.g., a conservative BoNT/C1 enzymatic domain variant, a non-conservative BoNT/C1 enzymatic domain variant, an active BoNT/C1 enzymatic domain fragment, or any combination thereof.

In other aspects of this embodiment, a BoNT/C1 enzymatic domain comprises a polypeptide having an amino acid identity of, e.g., at least 70%, at least 75%, at least 80%, at least 85%, at least 90%, or at least 95% to the enzymatic domain of SEQ ID NO: 11 or SEQ ID NO: 12; or at most 70%, at most 75%, at most 80%, at most 85%, at most 90%, or at most 95% to the enzymatic domain of SEQ ID NO: 11 or SEQ ID NO: 12. In yet other aspects of this embodiment, a BoNT/C1 enzymatic domain comprises a polypeptide having an amino acid identity of, e.g., at least 70%, at least 75%, at least 80%, at least 85%, at least 90%, or at least 95% to amino acids ½-436 of SEQ ID NO: 11; or at most 70%, at most 75%, at most 80%, at most 85%, at most 90%, or at most 95% to amino acids ½-436 of SEQ ID NO: 11.

In other aspects of this embodiment, a BoNT/C1 enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 11 or SEQ ID NO: 12; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 11 or SEQ ID NO: 12. In yet other aspects of this embodiment, a BoNT/C1 enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 11; or at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 11. In still other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 11 or SEQ ID NO: 12; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 11 or SEQ ID NO: 12. In further other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 11; or at most 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 11.

In another embodiment, a Clostridial toxin enzymatic domain comprises a BoNT/D enzymatic domain. In an aspect of this embodiment, a BoNT/D enzymatic domain comprises the enzymatic domains of SEQ ID NO: 13 or SEQ ID NO: 14. In other aspects of this embodiment, a BoNT/D enzymatic domain comprises amino acids ½-436 of SEQ ID NO: 13. In another aspect of this embodiment, a BoNT/D enzymatic domain comprises a naturally occurring BoNT/D enzymatic domain variant, such as, e.g., an enzymatic domain from a BoNT/D isoform or an enzymatic domain from a BoNT/D subtype. In another aspect of this embodiment, a BoNT/D enzymatic domain comprises a naturally occurring BoNT/D enzymatic domain variant of SEQ ID NO: 13 or SEQ ID NO: 14, such as, e.g., a BoNT/D isoform enzymatic domain or a BoNT/D subtype enzymatic domain. In another aspect of this embodiment, a BoNT/D enzymatic domain comprises amino acids ½-436 of a naturally occurring BoNT/D enzymatic domain variant of SEQ ID NO: 13, such as, e.g., a BoNT/D isoform enzymatic domain or a BoNT/D subtype enzymatic domain. In still another aspect of this embodiment, a BoNT/D enzymatic domain comprises a non-naturally occurring BoNT/D enzymatic domain variant, such as, e.g., a conservative BoNT/D enzymatic domain variant, a non-conservative BoNT/D enzymatic domain variant, an active BoNT/D enzymatic domain fragment, or any combination thereof. In still another aspect of this embodiment, a BoNT/D enzymatic domain comprises the enzymatic domain of a non-naturally occurring BoNT/D enzymatic domain variant of SEQ ID NO: 13 or SEQ ID NO: 14, such as, e.g., a conservative BoNT/D enzymatic domain variant, a non-conservative BoNT/D enzymatic domain variant, an active BoNT/D enzymatic domain fragment, or any combination thereof. In still another aspect of this embodiment, a BoNT/D enzymatic domain comprises amino acids ½-436 of a non-naturally occurring BoNT/D enzymatic domain variant of SEQ ID NO: 13, such as, e.g., a conservative BoNT/D enzymatic domain variant, a non-conservative BoNT/D enzymatic domain variant, an active BoNT/D enzymatic domain fragment, or any combination thereof.

In other aspects of this embodiment, a BoNT/D enzymatic domain comprises a polypeptide having an amino acid identity of, e.g., at least 70%, at least 75%, at least 80%, at least 85%, at least 90%, or at least 95% to the enzymatic domain of SEQ ID NO: 13 or SEQ ID NO: 14; or at most 70%, at most 75%, at most 80%, at most 85%, at most 90%, or at most 95% to the enzymatic domain of SEQ ID NO: 13 or SEQ ID NO: 14. In yet other aspects of this embodiment, a BoNT/D enzymatic domain comprises a polypeptide having an amino acid identity of, e.g., at least 70%, at least 75%, at least 80%, at least 85%, at least 90%, or at least 95% to amino acids ½-436 of SEQ ID NO: 13; or at most 70%, at most 75%, at most 80%, at most 85%, at most 90%, or at most 95% to amino acids ½-436 of SEQ ID NO: 13.

In other aspects of this embodiment, a BoNT/D enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 13 or SEQ ID NO: 14; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 13 or SEQ ID NO: 14. In yet other aspects of this embodiment, a BoNT/D enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 13; or at most 1, 2, 3, 4, 5, 6, 7, 8, 9, 10, 20, 30, 40, 50, or 100 non-contiguous amino acid deletions, additions, and/or substitutions relative to amino acids ½-436 of SEQ ID NO: 13. In still other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 13 or SEQ ID NO: 14; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 13 or SEQ ID NO: 14. In further other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 13; or at most 1, 2, 3, 4, 5, 6, 7, 8, 9, 10, 20, 30, 40, 50, or 100 contiguous amino acid deletions, additions, and/or substitutions relative to amino acids ½-436 of SEQ ID NO: 13.

In another embodiment, a Clostridial toxin enzymatic domain comprises a BoNT/E enzymatic domain. In an aspect of this embodiment, a BoNT/E enzymatic domain comprises the enzymatic domains of SEQ ID NO: 15, SEQ ID NO: 16, or SEQ ID NO: 17. In other aspects of this embodiment, a BoNT/E enzymatic domain comprises amino acids ½-411 of SEQ ID NO: 15. In another aspect of this embodiment, a BoNT/E enzymatic domain comprises a naturally occurring BoNT/E enzymatic domain variant, such as, e.g., an enzymatic domain from a BoNT/E isoform or an enzymatic domain from a BoNT/E subtype. In another aspect of this embodiment, a BoNT/E enzymatic domain comprises a naturally occurring BoNT/E enzymatic domain variant of SEQ ID NO: 15, SEQ ID NO: 16, or SEQ ID NO: 17, such as, e.g., a BoNT/E isoform enzymatic domain or a BoNT/E subtype enzymatic domain. In another aspect of this embodiment, a BoNT/E enzymatic domain comprises amino acids ½-411 of a naturally occurring BoNT/E enzymatic domain variant of SEQ ID NO: 15, such as, e.g., a BoNT/E isoform enzymatic domain or a BoNT/E subtype enzymatic domain. In still another aspect of this embodiment, a BoNT/E enzymatic domain comprises a non-naturally occurring BoNT/E enzymatic domain variant, such as, e.g., a conservative BoNT/E enzymatic domain variant, a non-conservative BoNT/E enzymatic domain variant, an active BoNT/E enzymatic domain fragment, or any combination thereof. In still another aspect of this embodiment, a BoNT/E enzymatic domain comprises the enzymatic domain of a non-naturally occurring BoNT/E enzymatic domain variant of SEQ ID NO: 15, SEQ ID NO: 16, or SEQ ID NO: 17, such as, e.g., a conservative BoNT/E enzymatic domain variant, a non-conservative BoNT/E enzymatic domain variant, an active BoNT/E enzymatic domain fragment, or any combination thereof. In still another aspect of this embodiment, a BoNT/E enzymatic domain comprises amino acids ½-411 of a non-naturally occurring BoNT/E enzymatic domain variant of SEQ ID NO: 15, such as, e.g., a conservative BoNT/E enzymatic domain variant, a non-conservative BoNT/E enzymatic domain variant, an active BoNT/E enzymatic domain fragment, or any combination thereof.

In other aspects of this embodiment, a BoNT/E enzymatic domain comprises a polypeptide having an amino acid identity of, e.g., at least 70%, at least 75%, at least 80%, at least 85%, at least 90%, or at least 95% to the enzymatic domain of SEQ ID NO: 15, SEQ ID NO: 16, or SEQ ID NO: 17; or at most 70%, at most 75%, at most 80%, at most 85%, at most 90%, or at most 95% to the enzymatic domain of SEQ ID NO: 15, SEQ ID NO: 16, or SEQ ID NO: 17. In yet other aspects of this embodiment, a BoNT/E enzymatic domain comprises a polypeptide having an amino acid identity of, e.g., at least 70%, at least 75%, at least 80%, at least 85%, at least 90%, or at least 95% to amino acids ½-411 of SEQ ID NO: 15; or at most 70%, at most 75%, at most 80%, at most 85%, at most 90%, or at most 95% to amino acids ½-411 of SEQ ID NO: 15.

In other aspects of this embodiment, a BoNT/E enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 15, SEQ ID NO: 16, or SEQ ID NO: 17; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 15, SEQ ID NO: 16, or SEQ ID NO: 17. In yet other aspects of this embodiment, a BoNT/E enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-411 of SEQ ID NO: 15; or at most 1, 2, 3, 4, 5, 6, 7, 8, 9, 10, 20, 30, 40, 50, or 100 non-contiguous amino acid deletions, additions, and/or substitutions relative to amino acids ½-411 of SEQ ID NO: 15. In still other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 15, SEQ ID NO: 16, or SEQ ID NO: 17; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 15, SEQ ID NO: 16, or SEQ ID NO: 17. In further other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-411 of SEQ ID NO: 15; or at most 1, 2, 3, 4, 5, 6, 7, 8, 9, 10, 20, 30, 40, 50, or 100 contiguous amino acid deletions, additions, and/or substitutions relative to amino acids ½-411 of SEQ ID NO: 15.

In another embodiment, a Clostridial toxin enzymatic domain comprises a BoNT/F enzymatic domain. In an aspect of this embodiment, a BoNT/F enzymatic domain comprises the enzymatic domains of SEQ ID NO: 18, SEQ ID NO: 19, or SEQ ID NO: 20. In other aspects of this embodiment, a BoNT/F enzymatic domain comprises amino acids ½-428 of SEQ ID NO: 18. In another aspect of this embodiment, a BoNT/F enzymatic domain comprises a naturally occurring BoNT/F enzymatic domain variant, such as, e.g., an enzymatic domain from a BoNT/F isoform or an enzymatic domain from a BoNT/F subtype. In another aspect of this embodiment, a BoNT/F enzymatic domain comprises a naturally occurring BoNT/F enzymatic domain variant of SEQ ID NO: 18, SEQ ID NO: 19, or SEQ ID NO: 20, such as, e.g., a BoNT/F isoform enzymatic domain or a BoNT/F subtype enzymatic domain. In another aspect of this embodiment, a BoNT/F enzymatic domain comprises amino acids ½-428 of a naturally occurring BoNT/F enzymatic domain variant of SEQ ID NO: 18, such as, e.g., a BoNT/F isoform enzymatic domain or a BoNT/F subtype enzymatic domain. In still another aspect of this embodiment, a BoNT/F enzymatic domain comprises a non-naturally occurring BoNT/F enzymatic domain variant, such as, e.g., a conservative BoNT/F enzymatic domain variant, a non-conservative BoNT/F enzymatic domain variant, an active BoNT/F enzymatic domain fragment, or any combination thereof. In still another aspect of this embodiment, a BoNT/F enzymatic domain comprises the enzymatic domain of a non-naturally occurring BoNT/F enzymatic domain variant of SEQ ID NO: 18, SEQ ID NO: 19, or SEQ ID NO: 20, such as, e.g., a conservative BoNT/F enzymatic domain variant, a non-conservative BoNT/F enzymatic domain variant, an active BoNT/F enzymatic domain fragment, or any combination thereof. In still another aspect of this embodiment, a BoNT/F enzymatic domain comprises amino acids ½-428 of a non-naturally occurring BoNT/F enzymatic domain variant of SEQ ID NO: 18, such as, e.g., a conservative BoNT/F enzymatic domain variant, a non-conservative BoNT/F enzymatic domain variant, an active BoNT/F enzymatic domain fragment, or any combination thereof.

In other aspects of this embodiment, a BoNT/F enzymatic domain comprises a polypeptide having an amino acid identity of, e.g., at least 70%, at least 75%, at least 80%, at least 85%, at least 90%, or at least 95% to the enzymatic domain of SEQ ID NO: 18, SEQ ID NO: 19, or SEQ ID NO: 20; or at most 70%, at most 75%, at most 80%, at most 85%, at most 90%, or at most 95% to the enzymatic domain of SEQ ID NO: 18, SEQ ID NO: 19, or SEQ ID NO: 20. In yet other aspects of this embodiment, a BoNT/F enzymatic domain comprises a polypeptide having an amino acid identity of, e.g., at least 70%, at least 75%, at least 80%, at least 85%, at least 90%, or at least 95% to amino acids ½-428 of SEQ ID NO: 18; or at most 70%, at most 75%, at most 80%, at most 85%, at most 90%, or at most 95% to amino acids ½-428 of SEQ ID NO: 18.

In other aspects of this embodiment, a BoNT/F enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 18, SEQ ID NO: 19, or SEQ ID NO: 20; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 18, SEQ ID NO: 19, or SEQ ID NO: 20. In yet other aspects of this embodiment, a BoNT/F enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-428 of SEQ ID NO: 18; or at most 1, 2, 3, 4, 5, 6, 7, 8, 9, 10, 20, 30, 40, 50, or 100 non-contiguous amino acid deletions, additions, and/or substitutions relative to amino acids ½-428 of SEQ ID NO: 18. In still other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 18, SEQ ID NO: 19, or SEQ ID NO: 20; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 18, SEQ ID NO: 19, or SEQ ID NO: 20. In further other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-428 of SEQ ID NO: 18; or at most 1, 2, 3, 4, 5, 6, 7, 8, 9, 10, 20, 30, 40, 50, or 100 contiguous amino acid deletions, additions, and/or substitutions relative to amino acids ½-428 of SEQ ID NO: 18.

In another embodiment, a Clostridial toxin enzymatic domain comprises a BoNT/G enzymatic domain. In an aspect of this embodiment, a BoNT/G enzymatic domain comprises the enzymatic domains of SEQ ID NO: 21. In other aspects of this embodiment, a BoNT/G enzymatic domain comprises amino acids ½-4435 of SEQ ID NO: 21. In another aspect of this embodiment, a BoNT/G enzymatic domain comprises a naturally occurring BoNT/G enzymatic domain variant, such as, e.g., an enzymatic domain from a BoNT/G isoform or an enzymatic domain from a BoNT/G subtype. In another aspect of this embodiment, a BoNT/G enzymatic domain comprises a naturally occurring BoNT/G enzymatic domain variant of SEQ ID NO: 21, such as, e.g., a BoNT/G isoform enzymatic domain or a BoNT/G subtype enzymatic domain. In another aspect of this embodiment, a BoNT/G enzymatic domain comprises amino acids ½-4435 of a naturally occurring BoNT/G enzymatic domain variant of SEQ ID NO: 21, such as, e.g., a BoNT/G isoform enzymatic domain or a BoNT/G subtype enzymatic domain. In still another aspect of this embodiment, a BoNT/G enzymatic domain comprises a non-naturally occurring BoNT/G enzymatic domain variant, such as, e.g., a conservative BoNT/G enzymatic domain variant, a non-conservative BoNT/G enzymatic domain variant, an active BoNT/G enzymatic domain fragment, or any combination thereof. In still another aspect of this embodiment, a BoNT/G enzymatic domain comprises the enzymatic domain of a non-naturally occurring BoNT/G enzymatic domain variant of SEQ ID NO: 21, such as, e.g., a conservative BoNT/G enzymatic domain variant, a non-conservative BoNT/G enzymatic domain variant, an active BoNT/G enzymatic domain fragment, or any combination thereof. In still another aspect of this embodiment, a BoNT/G enzymatic domain comprises amino acids ½-4435 of a non-naturally occurring BoNT/G enzymatic domain variant of SEQ ID NO: 21, such as, e.g., a conservative BoNT/G enzymatic domain variant, a non-conservative BoNT/G enzymatic domain variant, an active BoNT/G enzymatic domain fragment, or any combination thereof.

In other aspects of this embodiment, a BoNT/G enzymatic domain comprises a polypeptide having an amino acid identity of, e.g., at least 70%, at least 75%, at least 80%, at least 85%, at least 90%, or at least 95% to the enzymatic domain of SEQ ID NO: 21; or at most 70%, at most 75%, at most 80%, at most 85%, at most 90%, or at most 95% to the enzymatic domain of SEQ ID NO: 21. In yet other aspects of this embodiment, a BoNT/G enzymatic domain comprises a polypeptide having an amino acid identity of, e.g., at least 70%, at least 75%, at least 80%, at least 85%, at least 90%, or at least 95% to amino acids ½-4435 of SEQ ID NO: 21; or at most 70%, at most 75%, at most 80%, at most 85%, at most 90%, or at most 95% to amino acids ½-4435 of SEQ ID NO: 21.

In other aspects of this embodiment, a BoNT/G enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 21; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 21. In yet other aspects of this embodiment, a BoNT/G enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-4435 of SEQ ID NO: 21; or at most 1, 2, 3, 4, 5, 6, 7, 8, 9, 10, 20, 30, 40, 50, or 100 non-contiguous amino acid deletions, additions, and/or substitutions relative to amino acids ½-4435 of SEQ ID NO: 21. In still other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 21; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 21. In further other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-4435 of SEQ ID NO: 21; or at most 1, 2, 3, 4, 5, 6, 7, 8, 9, 10, 20, 30, 40, 50, or 100 contiguous amino acid deletions, additions, and/or substitutions relative to amino acids ½-4435 of SEQ ID NO: 21.

In another embodiment, a Clostridial toxin enzymatic domain comprises a TeNT enzymatic domain. In an aspect of this embodiment, a TeNT enzymatic domain comprises the enzymatic domains of SEQ ID NO: 22. In other aspects of this embodiment, a TeNT enzymatic domain comprises amino acids ½-438 of SEQ ID NO: 22. In another aspect of this embodiment, a TeNT enzymatic domain comprises a naturally occurring TeNT enzymatic domain variant, such as, e.g., an enzymatic domain from a TeNT isoform or an enzymatic domain from a TeNT subtype. In another aspect of this embodiment, a TeNT enzymatic domain comprises a naturally occurring TeNT enzymatic domain variant of SEQ ID NO: 22, such as, e.g., a TeNT isoform enzymatic domain or a TeNT subtype enzymatic domain. In another aspect of this embodiment, a TeNT enzymatic domain comprises amino acids ½-438 of a naturally occurring TeNT enzymatic domain variant of SEQ ID NO: 22, such as, e.g., a TeNT isoform enzymatic domain or a TeNT subtype enzymatic domain. In still another aspect of this embodiment, a TeNT enzymatic domain comprises a non-naturally occurring TeNT enzymatic domain variant, such as, e.g., a conservative TeNT enzymatic domain variant, a non-conservative TeNT enzymatic domain variant, an active TeNT enzymatic domain fragment, or any combination thereof. In still another aspect of this embodiment, a TeNT enzymatic domain comprises the enzymatic domain of a non-naturally occurring TeNT enzymatic domain variant of SEQ ID NO: 22, such as, e.g., a conservative TeNT enzymatic domain variant, a non-conservative TeNT enzymatic domain variant, an active TeNT enzymatic domain fragment, or any combination thereof. In still another aspect of this embodiment, a TeNT enzymatic domain comprises amino acids ½-438 of a non-naturally occurring TeNT enzymatic domain variant of SEQ ID NO: 22, such as, e.g., a conservative TeNT enzymatic domain variant, a non-conservative TeNT enzymatic domain variant, an active TeNT enzymatic domain fragment, or any combination thereof.

In other aspects of this embodiment, a TeNT enzymatic domain comprises a polypeptide having an amino acid identity of, e.g., at least 70%, at least 75%, at least 80%, at least 85%, at least 90%, or at least 95% to the enzymatic domain of SEQ ID NO: 22; or at most 70%, at most 75%, at most 80%, at most 85%, at most 90%, or at most 95% to the enzymatic domain of SEQ ID NO: 22. In yet other aspects of this embodiment, a TeNT enzymatic domain comprises a polypeptide having an amino acid identity of, e.g., at least 70%, at least 75%, at least 80%, at least 85%, at least 90%, or at least 95% to amino acids ½-438 of SEQ ID NO: 22; or at most 70%, at most 75%, at most 80%, at most 85%, at most 90%, or at most 95% to amino acids ½-438 of SEQ ID NO: 22.

In other aspects of this embodiment, a TeNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 22; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 22. In yet other aspects of this embodiment, a TeNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-438 of SEQ ID NO: 22; or at most 1, 2, 3, 4, 5, 6, 7, 8, 9, 10, 20, 30, 40, 50, or 100 non-contiguous amino acid deletions, additions, and/or substitutions relative to amino acids ½-438 of SEQ ID NO: 22. In still other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 22; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 22. In further other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-438 of SEQ ID NO: 22; or at most 1, 2, 3, 4, 5, 6, 7, 8, 9, 10, 20, 30, 40, 50, or 100 contiguous amino acid deletions, additions, and/or substitutions relative to amino acids ½-438 of SEQ ID NO: 22.

In another embodiment, a Clostridial toxin enzymatic domain comprises a BaNT enzymatic domain. In an aspect of this embodiment, a BaNT enzymatic domain comprises the enzymatic domains of SEQ ID NO: 23. In other aspects of this embodiment, a BaNT enzymatic domain comprises amino acids ½-420 of SEQ ID NO: 23. In another aspect of this embodiment, a BaNT enzymatic domain comprises a naturally occurring BaNT enzymatic domain variant, such as, e.g., an enzymatic domain from a BaNT isoform or an enzymatic domain from a BaNT subtype. In another aspect of this embodiment, a BaNT enzymatic domain comprises a naturally occurring BaNT enzymatic domain variant of SEQ ID NO: 23, such as, e.g., a BaNT isoform enzymatic domain or a BaNT subtype enzymatic domain. In another aspect of this embodiment, a BaNT enzymatic domain comprises amino acids ½-420 of a naturally occurring BaNT enzymatic domain variant of SEQ ID NO: 23, such as, e.g., a BaNT isoform enzymatic domain or a BaNT subtype enzymatic domain. In still another aspect of this embodiment, a BaNT enzymatic domain comprises a non-naturally occurring BaNT enzymatic domain variant, such as, e.g., a conservative BaNT enzymatic domain variant, a non-conservative BaNT enzymatic domain variant, an active BaNT enzymatic domain fragment, or any combination thereof. In still another aspect of this embodiment, a BaNT enzymatic domain comprises the enzymatic domain of a non-naturally occurring BaNT enzymatic domain variant of SEQ ID NO: 23, such as, e.g., a conservative BaNT enzymatic domain variant, a non-conservative BaNT enzymatic domain variant, an active BaNT enzymatic domain fragment, or any combination thereof. In still another aspect of this embodiment, a BaNT enzymatic domain comprises amino acids ½-420 of a non-naturally occurring BaNT enzymatic domain variant of SEQ ID NO: 23, such as, e.g., a conservative BaNT enzymatic domain variant, a non-conservative BaNT enzymatic domain variant, an active BaNT enzymatic domain fragment, or any combination thereof.

In other aspects of this embodiment, a BaNT enzymatic domain comprises a polypeptide having an amino acid identity of, e.g., at least 70%, at least 75%, at least 80%, at least 85%, at least 90%, or at least 95% to the enzymatic domain of SEQ ID NO: 23; or at most 70%, at most 75%, at most 80%, at most 85%, at most 90%, or at most 95% to the enzymatic domain of SEQ ID NO: 23. In yet other aspects of this embodiment, a BaNT enzymatic domain comprises a polypeptide having an amino acid identity of, e.g., at least 70%, at least 75%, at least 80%, at least 85%, at least 90%, or at least 95% to amino acids ½-420 of SEQ ID NO: 23; or at most 70%, at most 75%, at most 80%, at most 85%, at most 90%, or at most 95% to amino acids ½-420 of SEQ ID NO: 23.

In other aspects of this embodiment, a BaNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 23; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 23. In yet other aspects of this embodiment, a BaNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-420 of SEQ ID NO: 23; or at most 1, 2, 3, 4, 5, 6, 7, 8, 9, 10, 20, 30, 40, 50, or 100 non-contiguous amino acid deletions, additions, and/or substitutions relative to amino acids ½-420 of SEQ ID NO: 23. In still other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 23; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 23. In further other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-420 of SEQ ID NO: 23; or at most 1, 2, 3, 4, 5, 6, 7, 8, 9, 10, 20, 30, 40, 50, or 100 contiguous amino acid deletions, additions, and/or substitutions relative to amino acids ½-420 of SEQ ID NO: 23.

In another embodiment, a Clostridial toxin enzymatic domain comprises a BuNT enzymatic domain. In an aspect of this embodiment, a BuNT enzymatic domain comprises the enzymatic domains of SEQ ID NO: 24 or SEQ ID NO: 25. In other aspects of this embodiment, a BuNT enzymatic domain comprises amino acids ½-411 of SEQ ID NO: 24. In another aspect of this embodiment, a BuNT enzymatic domain comprises a naturally occurring BuNT enzymatic domain variant, such as, e.g., an enzymatic domain from a BuNT isoform or an enzymatic domain from a BuNT subtype. In another aspect of this embodiment, a BuNT enzymatic domain comprises a naturally occurring BuNT enzymatic domain variant of SEQ ID NO: 24 or SEQ ID NO: 25, such as, e.g., a BuNT isoform enzymatic domain or a BuNT subtype enzymatic domain. In another aspect of this embodiment, a BuNT enzymatic domain comprises amino acids ½-411 of a naturally occurring BuNT enzymatic domain variant of SEQ ID NO: 24, such as, e.g., a BuNT isoform enzymatic domain or a BuNT subtype enzymatic domain. In still another aspect of this embodiment, a BuNT enzymatic domain comprises a non-naturally occurring BuNT enzymatic domain variant, such as, e.g., a conservative BuNT enzymatic domain variant, a non-conservative BuNT enzymatic domain variant, an active BuNT enzymatic domain fragment, or any combination thereof. In still another aspect of this embodiment, a BuNT enzymatic domain comprises the enzymatic domain of a non-naturally occurring BuNT enzymatic domain variant of SEQ ID NO: 24 or SEQ ID NO: 25, such as, e.g., a conservative BuNT enzymatic domain variant, a non-conservative BuNT enzymatic domain variant, an active BuNT enzymatic domain fragment, or any combination thereof. In still another aspect of this embodiment, a BuNT enzymatic domain comprises amino acids ½-411 of a non-naturally occurring BuNT enzymatic domain variant of SEQ ID NO: 24, such as, e.g., a conservative BuNT enzymatic domain variant, a non-conservative BuNT enzymatic domain variant, an active BuNT enzymatic domain fragment, or any combination thereof.

In other aspects of this embodiment, a BuNT enzymatic domain comprises a polypeptide having an amino acid identity of, e.g., at least 70%, at least 75%, at least 80%, at least 85%, at least 90%, or at least 95% to the enzymatic domain of SEQ ID NO: 24 or SEQ ID NO: 25; or at most 70%, at most 75%, at most 80%, at most 85%, at most 90%, or at most 95% to the enzymatic domain of SEQ ID NO: 24 or SEQ ID NO: 25. In yet other aspects of this embodiment, a BuNT enzymatic domain comprises a polypeptide having an amino acid identity of, e.g., at least 70%, at least 75%, at least 80%, at least 85%, at least 90%, or at least 95% to amino acids ½-411 of SEQ ID NO: 24 or SEQ ID NO: 25; or at most 70%, at most 75%, at most 80%, at most 85%, at most 90%, or at most 95% to amino acids ½-411 of SEQ ID NO: 24 or SEQ ID NO: 25.

In other aspects of this embodiment, a BuNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 24 OR SEQ ID NO: 25. In yet other aspects of this embodiment, a BuNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids ½-411 of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to amino acids ½-411 of SEQ ID NO: 24 or SEQ ID NO: 25. In still other aspects of this embodiment, a BuNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 24 or SEQ ID NO: 25. In further other aspects of this embodiment, a BuNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids ½-411 of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to amino acids ½-411 of SEQ ID NO: 24 or SEQ ID NO: 25.

The “translocation domain” comprises a portion of a Clostridial neurotoxin heavy chain having a translocation activity. By “translocation” is meant the ability to facilitate the transport of a polypeptide through a vesicular membrane, thereby exposing some or all of the polypeptide to the cytoplasm. In the various botulinum neurotoxins translocation is thought to involve an allosteric conformational change of the heavy chain caused by a decrease in pH within the endosome. This conformational change appears to involve and be mediated by the N terminal half of the heavy chain and to result in the formation of pores in the vesicular membrane; this change permits the movement of the proteolytic light chain from within the endosomal vesicle into the cytoplasm. See e.g., Lacy, et al., Nature Struct. Biol. 5:898-902 (October 1998).

The amino acid sequence of the translocation-mediating portion of the botulinum neurotoxin heavy chain is known to those of skill in the art; additionally, those amino acid residues within this portion that are known to be essential for conferring the translocation activity are also known. It would therefore be well within the ability of one of ordinary skill in the art, for example, to employ the naturally occurring N-terminal peptide half of the heavy chain of any of the various Clostridium tetanus or Clostridium botulinum neurotoxin subtypes as a translocation domain, or to design an analogous translocation domain by aligning the primary sequences of the N-terminal halves of the various heavy chains and selecting a consensus primary translocation sequence based on conserved amino acid, polarity, steric and hydrophobicity characteristics between the sequences.

Aspects of the present specification provide, in part, a TVEMP comprising a Clostridial toxin translocation domain. As used herein, the term “Clostridial toxin translocation domain” refers to any Clostridial toxin polypeptide that can execute the translocation step of the intoxication process that mediates Clostridial toxin light chain translocation. Thus, a Clostridial toxin translocation domain facilitates the movement of a Clostridial toxin light chain across a membrane and encompasses the movement of a Clostridial toxin light chain through the membrane an intracellular vesicle into the cytoplasm of a cell. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, active Clostridial toxin translocation domain fragments thereof, or any combination thereof.

As used herein, the term “Clostridial toxin translocation domain variant,” whether naturally-occurring or non-naturally-occurring, refers to a Clostridial toxin translocation domain that has at least one amino acid change from the corresponding region of the disclosed reference sequences (Table 1) and can be described in percent identity to the corresponding region of that reference sequence. Unless expressly indicated, Clostridial toxin translocation domain variants useful to practice disclosed embodiments are variants that execute the translocation step of the intoxication process that mediates Clostridial toxin light chain translocation. As non-limiting examples, a BoNT/A translocation domain variant will have at least one amino acid difference, such as, e.g., an amino acid substitution, deletion or addition, as compared to amino acids 455-873 of SEQ ID NO: 1; a BoNT/B translocation domain variant will have at least one amino acid difference, such as, e.g., an amino acid substitution, deletion or addition, as compared to amino acids 447-860 of SEQ ID NO: 6; a BoNT/C1 translocation domain variant will have at least one amino acid difference, such as, e.g., an amino acid substitution, deletion or addition, as compared to amino acids 454-868 of SEQ ID NO: 11; a BoNT/D translocation domain variant will have at least one amino acid difference, such as, e.g., an amino acid substitution, deletion or addition, as compared to amino acids 451-864 of SEQ ID NO: 13; a BoNT/E translocation domain variant will have at least one amino acid difference, such as, e.g., an amino acid substitution, deletion or addition, as compared to amino acids 427-847 of SEQ ID NO: 15; a BoNT/F translocation domain variant will have at least one amino acid difference, such as, e.g., an amino acid substitution, deletion or addition, as compared to amino acids 446-865 of SEQ ID NO: 18; a BoNT/G translocation domain variant will have at least one amino acid difference, such as, e.g., an amino acid substitution, deletion or addition, as compared to amino acids 451-865 of SEQ ID NO: 21; a TeNT translocation domain variant will have at least one amino acid difference, such as, e.g., an amino acid substitution, deletion or addition, as compared to amino acids 468-881 of SEQ ID NO: 22; a BaNT translocation domain variant will have at least one amino acid difference, such as, e.g., an amino acid substitution, deletion or addition, as compared to amino acids 436-857 of SEQ ID NO: 23; and a BuNT translocation domain variant will have at least one amino acid difference, such as, e.g., an amino acid substitution, deletion or addition, as compared to amino acids 427-847 of SEQ ID NO: 24.

It is recognized by those of skill in the art that within each serotype of Clostridial toxin there can be naturally occurring Clostridial toxin translocation domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific translocation domain subtypes showing about 85-87% amino acid identity when compared to the BoNT/A translocation domain subtype of SEQ ID NO: 1. As used herein, the term “naturally occurring Clostridial toxin translocation domain variant” refers to any Clostridial toxin translocation domain produced by a naturally-occurring process, including, without limitation, Clostridial toxin translocation domain isoforms produced from alternatively-spliced transcripts, Clostridial toxin translocation domain isoforms produced by spontaneous mutation and Clostridial toxin translocation domain subtypes. A naturally occurring Clostridial toxin translocation domain variant can function in substantially the same manner as the reference Clostridial toxin translocation domain on which the naturally occurring Clostridial toxin translocation domain variant is based, and can be substituted for the reference Clostridial toxin translocation domain in any aspect of the present specification.

A non-limiting examples of a naturally occurring Clostridial toxin translocation domain variant is a Clostridial toxin translocation domain isoform such as, e.g., a BoNT/A translocation domain isoform, a BoNT/B translocation domain isoform, a BoNT/C1 translocation domain isoform, a BoNT/D translocation domain isoform, a BoNT/E translocation domain isoform, a BoNT/F translocation domain isoform, a BoNT/G translocation domain isoform, a TeNT translocation domain isoform, a BaNT translocation domain isoform, and a BuNT translocation domain isoform. Another non-limiting examples of a naturally occurring Clostridial toxin translocation domain variant is a Clostridial toxin translocation domain subtype such as, e.g., a translocation domain from subtype BoNT/A1, BoNT/A2, BoNT/A3, BoNT/A4, and BoNT/A5; a translocation domain from subtype BoNT/B1, BoNT/B2, BoNT/B bivalent and BoNT/B nonproteolytic; a translocation domain from subtype BoNT/C1-1 and BoNT/C1-2; a translocation domain from subtype BoNT/E1, BoNT/E2 and BoNT/E3; a translocation domain from subtype BoNT/F1, BoNT/F2, BoNT/F3; and a translocation domain from subtype BuNT-1 and BuNT-2.

As used herein, the term “non-naturally occurring Clostridial toxin translocation domain variant” refers to any Clostridial toxin translocation domain produced with the aid of human manipulation, including, without limitation, Clostridial toxin translocation domains produced by genetic engineering using random mutagenesis or rational design and Clostridial toxin translocation domains produced by chemical synthesis. Non-limiting examples of non-naturally occurring Clostridial toxin translocation domain variants include, e.g., conservative Clostridial toxin translocation domain variants, non-conservative Clostridial toxin translocation domain variants, and active Clostridial toxin translocation domain fragments.

As used herein, the term “conservative Clostridial toxin translocation domain variant” refers to a Clostridial toxin translocation domain that has at least one amino acid substituted by another amino acid or an amino acid analog that has at least one property similar to that of the original amino acid from the reference Clostridial toxin translocation domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin translocation domain variant can function in substantially the same manner as the reference Clostridial toxin translocation domain on which the conservative Clostridial toxin translocation domain variant is based, and can be substituted for the reference Clostridial toxin translocation domain in any aspect of the present specification. Non-limiting examples of a conservative Clostridial toxin translocation domain variant include, e.g., conservative BoNT/A translocation domain variants, conservative BoNT/B translocation domain variants, conservative BoNT/C1 translocation domain variants, conservative BoNT/D translocation domain variants, conservative BoNT/E translocation domain variants, conservative BoNT/F translocation domain variants, conservative BoNT/G translocation domain variants, conservative TeNT translocation domain variants, conservative BaNT translocation domain variants, and conservative BuNT translocation domain variants.

As used herein, the term “non-conservative Clostridial toxin translocation domain variant” refers to a Clostridial toxin translocation domain in which 1) at least one amino acid is deleted from the reference Clostridial toxin translocation domain on which the non-conservative Clostridial toxin translocation domain variant is based; 2) at least one amino acid added to the reference Clostridial toxin translocation domain on which the non-conservative Clostridial toxin translocation domain is based; or 3) at least one amino acid is substituted by another amino acid or an amino acid analog that does not share any property similar to that of the original amino acid from the reference Clostridial toxin translocation domain sequence (Table 1). A non-conservative Clostridial toxin translocation domain variant can function in substantially the same manner as the reference Clostridial toxin translocation domain on which the non-conservative Clostridial toxin translocation domain variant is based, and can be substituted for the reference Clostridial toxin translocation domain in any aspect of the present specification. Non-limiting examples of a non-conservative Clostridial toxin translocation domain variant include, e.g., non-conservative BoNT/A translocation domain variants, non-conservative BoNT/B translocation domain variants, non-conservative BoNT/C1 translocation domain variants, non-conservative BoNT/D translocation domain variants, non-conservative BoNT/E translocation domain variants, non-conservative BoNT/F translocation domain variants, non-conservative BoNT/G translocation domain variants, and non-conservative TeNT translocation domain variants, non-conservative BaNT translocation domain variants, and non-conservative BuNT translocation domain variants.

As used herein, the term “active Clostridial toxin translocation domain fragment” refers to any of a variety of Clostridial toxin fragments comprising the translocation domain can be useful in aspects of the present specification with the proviso that these active fragments can facilitate the release of the LC from intracellular vesicles into the cytoplasm of the target cell and thus participate in executing the overall cellular mechanism whereby a Clostridial toxin proteolytically cleaves a substrate. The translocation domains from the heavy chains of Clostridial toxins are approximately 410-430 amino acids in length and comprise a translocation domain (Table 1). Research has shown that the entire length of a translocation domain from a Clostridial toxin heavy chain is not necessary for the translocating activity of the translocation domain. Thus, aspects of this embodiment include a Clostridial toxin translocation domain having a length of, e.g., at least 350, 375, 400, or 425 amino acids. Other aspects of this embodiment include a Clostridial toxin translocation domain having a length of, e.g., at most 350, 375, 400, or 425 amino acids.

Any of a variety of sequence alignment methods can be used to determine percent identity of naturally-occurring Clostridial toxin translocation domain variants and non-naturally-occurring Clostridial toxin translocation domain variants, including, without limitation, global methods, local methods and hybrid methods, such as, e.g., segment approach methods. Protocols to determine percent identity are routine procedures within the scope of one skilled in the art and from the teaching herein.

Thus, in an embodiment, a TVEMP disclosed herein comprises a Clostridial toxin translocation domain. In an aspect of this embodiment, a Clostridial toxin translocation domain comprises a naturally occurring Clostridial toxin translocation domain variant, such as, e.g., a Clostridial toxin translocation domain isoform or a Clostridial toxin translocation domain subtype. In another aspect of this embodiment, a Clostridial toxin translocation domain comprises a non-naturally occurring Clostridial toxin translocation domain variant, such as, e.g., a conservative Clostridial toxin translocation domain variant, a non-conservative Clostridial toxin translocation domain variant, an active Clostridial toxin translocation domain fragment, or any combination thereof.

In another embodiment, a hydrophic amino acid at one particular position in the polypeptide chain of the Clostridial toxin translocation domain can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin translocation domain can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin translocation domain can be substituted with another aromatic amino acid. Examples of aromatic amino acids include, e.g., F, H, W and Y. In still another aspect of this embodiment, a stacking amino acid at one particular position in the polypeptide chain of the Clostridial toxin translocation domain can be substituted with another stacking amino acid. Examples of stacking amino acids include, e.g., F, H, W and Y. In a further aspect of this embodiment, a polar amino acid at one particular position in the polypeptide chain of the Clostridial toxin translocation domain can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin translocation domain can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin translocation domain can be substituted with another positive charged amino acid. Examples of positive charged amino acids include, e.g., K, R, and H. In a still further aspect of this embodiment, a negative charged amino acid at one particular position in the polypeptide chain of the Clostridial toxin translocation domain can be substituted with another negative charged amino acid. Examples of negative charged amino acids include, e.g., D and E. In another aspect of this embodiment, a small amino acid at one particular position in the polypeptide chain of the Clostridial toxin translocation domain can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin translocation domain can be substituted with another C-beta branching amino acid. Examples of C-beta branching amino acids include, e.g., I, T and V.

In another embodiment, a Clostridial toxin translocation domain comprises a BoNT/A translocation domain. In an aspect of this embodiment, a BoNT/A translocation domain comprises the translocation domains of SEQ ID NO: 1, SEQ ID NO: 2, SEQ ID NO: 3, SEQ ID NO: 4, or SEQ ID NO: 5. In other aspects of this embodiment, a BoNT/A translocation domain comprises amino acids 455-873 of SEQ ID NO: 1. In another aspect of this embodiment, a BoNT/A translocation domain comprises a naturally occurring BoNT/A translocation domain variant, such as, e.g., an translocation domain from a BoNT/A isoform or an translocation domain from a BoNT/A subtype. In another aspect of this embodiment, a BoNT/A translocation domain comprises a naturally occurring BoNT/A translocation domain variant of SEQ ID NO: 1, SEQ ID NO: 2, SEQ ID NO: 3, SEQ ID NO: 4, or SEQ ID NO: 5, such as, e.g., a BoNT/A isoform translocation domain or a BoNT/A subtype translocation domain. In another aspect of this embodiment, a BoNT/A translocation domain comprises amino acids 455-873 of a naturally occurring BoNT/A translocation domain variant of SEQ ID NO: 1, such as, e.g., a BoNT/A isoform translocation domain or a BoNT/A subtype translocation domain. In still another aspect of this embodiment, a BoNT/A translocation domain comprises a non-naturally occurring BoNT/A translocation domain variant, such as, e.g., a conservative BoNT/A translocation domain variant, a non-conservative BoNT/A translocation domain variant, an active BoNT/A translocation domain fragment, or any combination thereof. In still another aspect of this embodiment, a BoNT/A translocation domain comprises the translocation domain of a non-naturally occurring BoNT/A translocation domain variant of SEQ ID NO: 1, SEQ ID NO: 2, SEQ ID NO: 3, SEQ ID NO: 4, or SEQ ID NO: 5, such as, e.g., a conservative BoNT/A translocation domain variant, a non-conservative BoNT/A translocation domain variant, an active BoNT/A translocation domain fragment, or any combination thereof. In still another aspect of this embodiment, a BoNT/A translocation domain comprises amino acids 455-873 of a non-naturally occurring BoNT/A translocation domain variant of SEQ ID NO: 1, such as, e.g., a conservative BoNT/A translocation domain variant, a non-conservative BoNT/A translocation domain variant, an active BoNT/A translocation domain fragment, or any combination thereof.

In other aspects of this embodiment, a BoNT/A translocation domain comprises a polypeptide having an amino acid identity of, e.g., at least 70%, at least 75%, at least 80%, at least 85%, at least 90%, or at least 95% to the translocation domain of SEQ ID NO: 1, SEQ ID NO: 2, SEQ ID NO: 3, SEQ ID NO: 4, or SEQ ID NO: 5; or at most 70%, at most 75%, at most 80%, at most 85%, at most 90%, or at most 95% to the translocation domain of SEQ ID NO: 1, SEQ ID NO: 2, SEQ ID NO: 3, SEQ ID NO: 4, or SEQ ID NO: 5. In yet other aspects of this embodiment, a BoNT/A translocation domain comprises a polypeptide having an amino acid identity of, e.g., at least 70%, at least 75%, at least 80%, at least 85%, at least 90%, or at least 95% to amino acids 455-873 of SEQ ID NO: 1; or at most 70%, at most 75%, at most 80%, at most 85%, at most 90%, or at most 95% to amino acids 455-873 of SEQ ID NO: 1.

In other aspects of this embodiment, a BoNT/A translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5. In yet other aspects of this embodiment, a BoNT/A translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 455-873 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 455-873 of SEQ ID NO: 1. In still other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5. In further other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 455-873 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids 455-873 of SEQ ID NO: 1.

In another embodiment, a Clostridial toxin translocation domain comprises a BoNT/B translocation domain. In an aspect of this embodiment, a BoNT/B translocation domain comprises the translocation domains of SEQ ID NO: 6, SEQ ID NO: 7, SEQ ID NO: 8, SEQ ID NO: 9, or SEQ ID NO: 10. In other aspects of this embodiment, a BoNT/B translocation domain comprises amino acids 447-860 of SEQ ID NO: 6. In another aspect of this embodiment, a BoNT/B translocation domain comprises a naturally occurring BoNT/B translocation domain variant, such as, e.g., an translocation domain from a BoNT/B isoform or an translocation domain from a BoNT/B subtype. In another aspect of this embodiment, a BoNT/B translocation domain comprises a naturally occurring BoNT/B translocation domain variant of SEQ ID NO: 6, SEQ ID NO: 7, SEQ ID NO: 8, SEQ ID NO: 9, or SEQ ID NO: 10, such as, e.g., a BoNT/B isoform translocation domain or a BoNT/B subtype translocation domain. In another aspect of this embodiment, a BoNT/B translocation domain comprises amino acids 447-860 of a naturally occurring BoNT/B translocation domain variant of SEQ ID NO: 6, such as, e.g., a BoNT/B isoform translocation domain or a BoNT/B subtype translocation domain. In still another aspect of this embodiment, a BoNT/B translocation domain comprises a non-naturally occurring BoNT/B translocation domain variant, such as, e.g., a conservative BoNT/B translocation domain variant, a non-conservative BoNT/B translocation domain variant, an active BoNT/B translocation domain fragment, or any combination thereof. In still another aspect of this embodiment, a BoNT/B translocation domain comprises the translocation domain of a non-naturally occurring BoNT/B translocation domain variant of SEQ ID NO: 6, SEQ ID NO: 7, SEQ ID NO: 8, SEQ ID NO: 9, or SEQ ID NO: 10, such as, e.g., a conservative BoNT/B translocation domain variant, a non-conservative BoNT/B translocation domain variant, an active BoNT/B translocation domain fragment, or any combination thereof. In still another aspect of this embodiment, a BoNT/B translocation domain comprises amino acids 447-860 of a non-naturally occurring BoNT/B translocation domain variant of SEQ ID NO: 6, such as, e.g., a conservative BoNT/B translocation domain variant, a non-conservative BoNT/B translocation domain variant, an active BoNT/B translocation domain fragment, or any combination thereof.

In other aspects of this embodiment, a BoNT/B translocation domain comprises a polypeptide having an amino acid identity of, e.g., at least 70%, at least 75%, at least 80%, at least 85%, at least 90%, or at least 95% to the translocation domain of SEQ ID NO: 6, SEQ ID NO: 7, SEQ ID NO: 8, SEQ ID NO: 9, or SEQ ID NO: 10; or at most 70%, at most 75%, at most 80%, at most 85%, at most 90%, or at most 95% to the translocation domain of SEQ ID NO: 6, SEQ ID NO: 7, SEQ ID NO: 8, SEQ ID NO: 9, or SEQ ID NO: 10. In yet other aspects of this embodiment, a BoNT/B translocation domain comprises a polypeptide having an amino acid identity of, e.g., at least 70%, at least 75%, at least 80%, at least 85%, at least 90%, or at least 95% to amino acids 447-860 of SEQ ID NO: 6; or at most 70%, at most 75%, at most 80%, at most 85%, at most 90%, or at most 95% to amino acids 447-860 of SEQ ID NO: 6.

In other aspects of this embodiment, a BoNT/B translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10. In yet other aspects of this embodiment, a BoNT/B translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 447-860 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids 447-860 of SEQ ID NO: 6. In still other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10. In further other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 447-860 of SEQ ID NO: 6; or at most 1, 2, 3, 4, 5, 6, 7, 8, 9, 10, 20, 30, 40, 50, or 100 contiguous amino acid deletions, additions, and/or substitutions relative to amino acids 447-860 of SEQ ID NO: 6.

In another embodiment, a Clostridial toxin translocation domain comprises a BoNT/C1 translocation domain. In an aspect of this embodiment, a BoNT/C1 translocation domain comprises the translocation domains of SEQ ID NO: 11 or SEQ ID NO: 12. In other aspects of this embodiment, a BoNT/C1 translocation domain comprises amino acids 454-868 of SEQ ID NO: 11. In another aspect of this embodiment, a BoNT/C1 translocation domain comprises a naturally occurring BoNT/C1 translocation domain variant, such as, e.g., an translocation domain from a BoNT/C1 isoform or an translocation domain from a BoNT/C1 subtype. In another aspect of this embodiment, a BoNT/C1 translocation domain comprises a naturally occurring BoNT/C1 translocation domain variant of SEQ ID NO: 11 or SEQ ID NO: 12, such as, e.g., a BoNT/C1 isoform translocation domain or a BoNT/C1 subtype translocation domain. In another aspect of this embodiment, a BoNT/C1 translocation domain comprises amino acids 454-868 of a naturally occurring BoNT/C1 translocation domain variant of SEQ ID NO: 11, such as, e.g., a BoNT/C1 isoform translocation domain or a BoNT/C1 subtype translocation domain. In still another aspect of this embodiment, a BoNT/C1 translocation domain comprises a non-naturally occurring BoNT/C1 translocation domain variant, such as, e.g., a conservative BoNT/C1 translocation domain variant, a non-conservative BoNT/C1 translocation domain variant, an active BoNT/C1 translocation domain fragment, or any combination thereof. In still another aspect of this embodiment, a BoNT/C1 translocation domain comprises the translocation domain of a non-naturally occurring BoNT/C1 translocation domain variant of SEQ ID NO: 11 or SEQ ID NO: 12, such as, e.g., a conservative BoNT/C1 translocation domain variant, a non-conservative BoNT/C1 translocation domain variant, an active BoNT/C1 translocation domain fragment, or any combination thereof. In still another aspect of this embodiment, a BoNT/C1 translocation domain comprises amino acids 454-868 of a non-naturally occurring BoNT/C1 translocation domain variant of SEQ ID NO: 11, such as, e.g., a conservative BoNT/C1 translocation domain variant, a non-conservative BoNT/C1 translocation domain variant, an active BoNT/C1 translocation domain fragment, or any combination thereof.

In other aspects of this embodiment, a BoNT/C1 translocation domain comprises a polypeptide having an amino acid identity of, e.g., at least 70%, at least 75%, at least 80%, at least 85%, at least 90%, or at least 95% to the translocation domain of SEQ ID NO: 11 or SEQ ID NO: 12; or at most 70%, at most 75%, at most 80%, at most 85%, at most 90%, or at most 95% to the translocation domain of SEQ ID NO: 11 or SEQ ID NO: 12. In yet other aspects of this embodiment, a BoNT/C1 translocation domain comprises a polypeptide having an amino acid identity of, e.g., at least 70%, at least 75%, at least 80%, at least 85%, at least 90%, or at least 95% to amino acids 454-868 of SEQ ID NO: 11; or at most 70%, at most 75%, at most 80%, at most 85%, at most 90%, or at most 95% to amino acids 454-868 of SEQ ID NO: 11.

In other aspects of this embodiment, a BoNT/C1 translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 11 or SEQ ID NO: 12; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 11 or SEQ ID NO: 12. In yet other aspects of this embodiment, a BoNT/C1 translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 454-868 of SEQ ID NO: 11; or at most 1, 2, 3, 4, 5, 6, 7, 8, 9, 10, 20, 30, 40, 50, or 100 non-contiguous amino acid deletions, additions, and/or substitutions relative to amino acids 454-868 of SEQ ID NO: 11. In still other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 11 or SEQ ID NO: 12; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 11 or SEQ ID NO: 12. In further other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 454-868 of SEQ ID NO: 11; or at most 1, 2, 3, 4, 5, 6, 7, 8, 9, 10, 20, 30, 40, 50, or 100 contiguous amino acid deletions, additions, and/or substitutions relative to amino acids 454-868 of SEQ ID NO: 11.

In another embodiment, a Clostridial toxin translocation domain comprises a BoNT/D translocation domain. In an aspect of this embodiment, a BoNT/D translocation domain comprises the translocation domains of SEQ ID NO: 13 or SEQ ID NO: 14. In other aspects of this embodiment, a BoNT/D translocation domain comprises amino acids 451-864 of SEQ ID NO: 13. In another aspect of this embodiment, a BoNT/D translocation domain comprises a naturally occurring BoNT/D translocation domain variant, such as, e.g., an translocation domain from a BoNT/D isoform or an translocation domain from a BoNT/D subtype. In another aspect of this embodiment, a BoNT/D translocation domain comprises a naturally occurring BoNT/D translocation domain variant of SEQ ID NO: 13 or SEQ ID NO: 14, such as, e.g., a BoNT/D isoform translocation domain or a BoNT/D subtype translocation domain. In another aspect of this embodiment, a BoNT/D translocation domain comprises amino acids 451-864 of a naturally occurring BoNT/D translocation domain variant of SEQ ID NO: 13, such as, e.g., a BoNT/D isoform translocation domain or a BoNT/D subtype translocation domain. In still another aspect of this embodiment, a BoNT/D translocation domain comprises a non-naturally occurring BoNT/D translocation domain variant, such as, e.g., a conservative BoNT/D translocation domain variant, a non-conservative BoNT/D translocation domain variant, an active BoNT/D translocation domain fragment, or any combination thereof. In still another aspect of this embodiment, a BoNT/D translocation domain comprises the translocation domain of a non-naturally occurring BoNT/D translocation domain variant of SEQ ID NO: 13 or SEQ ID NO: 14, such as, e.g., a conservative BoNT/D translocation domain variant, a non-conservative BoNT/D translocation domain variant, an active BoNT/D translocation domain fragment, or any combination thereof. In still another aspect of this embodiment, a BoNT/D translocation domain comprises amino acids 451-864 of a non-naturally occurring BoNT/D translocation domain variant of SEQ ID NO: 13, such as, e.g., a conservative BoNT/D translocation domain variant, a non-conservative BoNT/D translocation domain variant, an active BoNT/D translocation domain fragment, or any combination thereof.

In other aspects of this embodiment, a BoNT/D translocation domain comprises a polypeptide having an amino acid identity of, e.g., at least 70%, at least 75%, at least 80%, at least 85%, at least 90%, or at least 95% to the translocation domain of SEQ ID NO: 13 or SEQ ID NO: 14; or at most 70%, at most 75%, at most 80%, at most 85%, at most 90%, or at most 95% to the translocation domain of SEQ ID NO: 13 or SEQ ID NO: 14. In yet other aspects of this embodiment, a BoNT/D translocation domain comprises a polypeptide having an amino acid identity of, e.g., at least 70%, at least 75%, at least 80%, at least 85%, at least 90%, or at least 95% to amino acids 451-864 of SEQ ID NO: 13; or at most 70%, at most 75%, at most 80%, at most 85%, at most 90%, or at most 95% to amino acids 451-864 of SEQ ID NO: 13.

In other aspects of this embodiment, a BoNT/D translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 13 or SEQ ID NO: 14; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 13 or SEQ ID NO: 14. In yet other aspects of this embodiment, a BoNT/D translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 451-864 of SEQ ID NO: 13; or at most 1, 2, 3, 4, 5, 6, 7, 8, 9, 10, 20, 30, 40, 50, or 100 non-contiguous amino acid deletions, additions, and/or substitutions relative to amino acids 451-864 of SEQ ID NO: 13. In still other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 13 or SEQ ID NO: 14; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 13 or SEQ ID NO: 14. In further other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 451-864 of SEQ ID NO: 13; or at most 1, 2, 3, 4, 5, 6, 7, 8, 9, 10, 20, 30, 40, 50, or 100 contiguous amino acid deletions, additions, and/or substitutions relative to amino acids 451-864 of SEQ ID NO: 13.

In another embodiment, a Clostridial toxin translocation domain comprises a BoNT/E translocation domain. In an aspect of this embodiment, a BoNT/E translocation domain comprises the translocation domains of SEQ ID NO: 15, SEQ ID NO: 16, or SEQ ID NO: 17. In other aspects of this embodiment, a BoNT/E translocation domain comprises amino acids 427-847 of SEQ ID NO: 15. In another aspect of this embodiment, a BoNT/E translocation domain comprises a naturally occurring BoNT/E translocation domain variant, such as, e.g., an translocation domain from a BoNT/E isoform or an translocation domain from a BoNT/E subtype. In another aspect of this embodiment, a BoNT/E translocation domain comprises a naturally occurring BoNT/E translocation domain variant of SEQ ID NO: 15, SEQ ID NO: 16, or SEQ ID NO: 17, such as, e.g., a BoNT/E isoform translocation domain or a BoNT/E subtype translocation domain. In another aspect of this embodiment, a BoNT/E translocation domain comprises amino acids 427-847 of a naturally occurring BoNT/E translocation domain variant of SEQ ID NO: 15, such as, e.g., a BoNT/E isoform translocation domain or a BoNT/E subtype translocation domain. In still another aspect of this embodiment, a BoNT/E translocation domain comprises a non-naturally occurring BoNT/E translocation domain variant, such as, e.g., a conservative BoNT/E translocation domain variant, a non-conservative BoNT/E translocation domain variant, an active BoNT/E translocation domain fragment, or any combination thereof. In still another aspect of this embodiment, a BoNT/E translocation domain comprises the translocation domain of a non-naturally occurring BoNT/E translocation domain variant of SEQ ID NO: 15, SEQ ID NO: 16, or SEQ ID NO: 17, such as, e.g., a conservative BoNT/E translocation domain variant, a non-conservative BoNT/E translocation domain variant, an active BoNT/E translocation domain fragment, or any combination thereof. In still another aspect of this embodiment, a BoNT/E translocation domain comprises amino acids 427-847 of a non-naturally occurring BoNT/E translocation domain variant of SEQ ID NO: 15, such as, e.g., a conservative BoNT/E translocation domain variant, a non-conservative BoNT/E translocation domain variant, an active BoNT/E translocation domain fragment, or any combination thereof.

In other aspects of this embodiment, a BoNT/E translocation domain comprises a polypeptide having an amino acid identity of, e.g., at least 70%, at least 75%, at least 80%, at least 85%, at least 90%, or at least 95% to the translocation domain of SEQ ID NO: 15, SEQ ID NO: 16, or SEQ ID NO: 17; or at most 70%, at most 75%, at most 80%, at most 85%, at most 90%, or at most 95% to the translocation domain of SEQ ID NO: 15, SEQ ID NO: 16, or SEQ ID NO: 17. In yet other aspects of this embodiment, a BoNT/E translocation domain comprises a polypeptide having an amino acid identity of, e.g., at least 70%, at least 75%, at least 80%, at least 85%, at least 90%, or at least 95% to amino acids 427-847 of SEQ ID NO: 15; or at most 70%, at most 75%, at most 80%, at most 85%, at most 90%, or at most 95% to amino acids 427-847 of SEQ ID NO: 15.

In other aspects of this embodiment, a BoNT/E translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 15, SEQ ID NO: 16, or SEQ ID NO: 17; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 15, SEQ ID NO: 16, or SEQ ID NO: 17. In yet other aspects of this embodiment, a BoNT/E translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 15; or at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 15. In still other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 15, SEQ ID NO: 16, or SEQ ID NO: 17; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 15, SEQ ID NO: 16, or SEQ ID NO: 17. In further other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 15; or at most 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 15.

In another embodiment, a Clostridial toxin translocation domain comprises a BoNT/F translocation domain. In an aspect of this embodiment, a BoNT/F translocation domain comprises the translocation domains of SEQ ID NO: 18, SEQ ID NO: 19, or SEQ ID NO: 20. In other aspects of this embodiment, a BoNT/F translocation domain comprises amino acids 446-865 of SEQ ID NO: 18. In another aspect of this embodiment, a BoNT/F translocation domain comprises a naturally occurring BoNT/F translocation domain variant, such as, e.g., an translocation domain from a BoNT/F isoform or an translocation domain from a BoNT/F subtype. In another aspect of this embodiment, a BoNT/F translocation domain comprises a naturally occurring BoNT/F translocation domain variant of SEQ ID NO: 18, SEQ ID NO: 19, or SEQ ID NO: 20, such as, e.g., a BoNT/F isoform translocation domain or a BoNT/F subtype translocation domain. In another aspect of this embodiment, a BoNT/F translocation domain comprises amino acids 446-865 of a naturally occurring BoNT/F translocation domain variant of SEQ ID NO: 18, such as, e.g., a BoNT/F isoform translocation domain or a BoNT/F subtype translocation domain. In still another aspect of this embodiment, a BoNT/F translocation domain comprises a non-naturally occurring BoNT/F translocation domain variant, such as, e.g., a conservative BoNT/F translocation domain variant, a non-conservative BoNT/F translocation domain variant, an active BoNT/F translocation domain fragment, or any combination thereof. In still another aspect of this embodiment, a BoNT/F translocation domain comprises the translocation domain of a non-naturally occurring BoNT/F translocation domain variant of SEQ ID NO: 18, SEQ ID NO: 19, or SEQ ID NO: 20, such as, e.g., a conservative BoNT/F translocation domain variant, a non-conservative BoNT/F translocation domain variant, an active BoNT/F translocation domain fragment, or any combination thereof. In still another aspect of this embodiment, a BoNT/F translocation domain comprises amino acids 446-865 of a non-naturally occurring BoNT/F translocation domain variant of SEQ ID NO: 18, such as, e.g., a conservative BoNT/F translocation domain variant, a non-conservative BoNT/F translocation domain variant, an active BoNT/F translocation domain fragment, or any combination thereof.

In other aspects of this embodiment, a BoNT/F translocation domain comprises a polypeptide having an amino acid identity of, e.g., at least 70%, at least 75%, at least 80%, at least 85%, at least 90%, or at least 95% to the translocation domain of SEQ ID NO: 18, SEQ ID NO: 19, or SEQ ID NO: 20; or at most 70%, at most 75%, at most 80%, at most 85%, at most 90%, or at most 95% to the translocation domain of SEQ ID NO: 18, SEQ ID NO: 19, or SEQ ID NO: 20. In yet other aspects of this embodiment, a BoNT/F translocation domain comprises a polypeptide having an amino acid identity of, e.g., at least 70%, at least 75%, at least 80%, at least 85%, at least 90%, or at least 95% to amino acids 446-865 of SEQ ID NO: 18; or at most 70%, at most 75%, at most 80%, at most 85%, at most 90%, or at most 95% to amino acids 446-865 of SEQ ID NO: 18.

In other aspects of this embodiment, a BoNT/F translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 18, SEQ ID NO: 19, or SEQ ID NO: 20; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 18, SEQ ID NO: 19, or SEQ ID NO: 20. In yet other aspects of this embodiment, a BoNT/F translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 446-865 of SEQ ID NO: 18; or at most 1, 2, 3, 4, 5, 6, 7, 8, 9, 10, 20, 30, 40, 50, or 100 non-contiguous amino acid deletions, additions, and/or substitutions relative to amino acids 446-865 of SEQ ID NO: 18. In still other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 18, SEQ ID NO: 19, or SEQ ID NO: 20; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 18, SEQ ID NO: 19, or SEQ ID NO: 20. In further other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 446-865 of SEQ ID NO: 18; or at most 1, 2, 3, 4, 5, 6, 7, 8, 9, 10, 20, 30, 40, 50, or 100 contiguous amino acid deletions, additions, and/or substitutions relative to amino acids 446-865 of SEQ ID NO: 18.

In another embodiment, a Clostridial toxin translocation domain comprises a BoNT/G translocation domain. In an aspect of this embodiment, a BoNT/G translocation domain comprises the translocation domains of SEQ ID NO: 21. In other aspects of this embodiment, a BoNT/G translocation domain comprises amino acids 451-865 of SEQ ID NO: 21. In another aspect of this embodiment, a BoNT/G translocation domain comprises a naturally occurring BoNT/G translocation domain variant, such as, e.g., an translocation domain from a BoNT/G isoform or an translocation domain from a BoNT/G subtype. In another aspect of this embodiment, a BoNT/G translocation domain comprises a naturally occurring BoNT/G translocation domain variant of SEQ ID NO: 21, such as, e.g., a BoNT/G isoform translocation domain or a BoNT/G subtype translocation domain. In another aspect of this embodiment, a BoNT/G translocation domain comprises amino acids 451-865 of a naturally occurring BoNT/G translocation domain variant of SEQ ID NO: 21, such as, e.g., a BoNT/G isoform translocation domain or a BoNT/G subtype translocation domain. In still another aspect of this embodiment, a BoNT/G translocation domain comprises a non-naturally occurring BoNT/G translocation domain variant, such as, e.g., a conservative BoNT/G translocation domain variant, a non-conservative BoNT/G translocation domain variant, an active BoNT/G translocation domain fragment, or any combination thereof. In still another aspect of this embodiment, a BoNT/G translocation domain comprises the translocation domain of a non-naturally occurring BoNT/G translocation domain variant of SEQ ID NO: 21, such as, e.g., a conservative BoNT/G translocation domain variant, a non-conservative BoNT/G translocation domain variant, an active BoNT/G translocation domain fragment, or any combination thereof. In still another aspect of this embodiment, a BoNT/G translocation domain comprises amino acids 451-865 of a non-naturally occurring BoNT/G translocation domain variant of SEQ ID NO: 21, such as, e.g., a conservative BoNT/G translocation domain variant, a non-conservative BoNT/G translocation domain variant, an active BoNT/G translocation domain fragment, or any combination thereof.

In other aspects of this embodiment, a BoNT/G translocation domain comprises a polypeptide having an amino acid identity of, e.g., at least 70%, at least 75%, at least 80%, at least 85%, at least 90%, or at least 95% to the translocation domain of SEQ ID NO: 21; or at most 70%, at most 75%, at most 80%, at most 85%, at most 90%, or at most 95% to the translocation domain of SEQ ID NO: 21. In yet other aspects of this embodiment, a BoNT/G translocation domain comprises a polypeptide having an amino acid identity of, e.g., at least 70%, at least 75%, at least 80%, at least 85%, at least 90%, or at least 95% to amino acids 451-865 of SEQ ID NO: 21; or at most 70%, at most 75%, at most 80%, at most 85%, at most 90%, or at most 95% to amino acids 451-865 of SEQ ID NO: 21.

In other aspects of this embodiment, a BoNT/G translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 21; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 21. In yet other aspects of this embodiment, a BoNT/G translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 451-865 of SEQ ID NO: 21; or at most 1, 2, 3, 4, 5, 6, 7, 8, 9, 10, 20, 30, 40, 50, or 100 non-contiguous amino acid deletions, additions, and/or substitutions relative to amino acids 451-865 of SEQ ID NO: 21. In still other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 21; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 21. In further other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 451-865 of SEQ ID NO: 21; or at most 1, 2, 3, 4, 5, 6, 7, 8, 9, 10, 20, 30, 40, 50, or 100 contiguous amino acid deletions, additions, and/or substitutions relative to amino acids 451-865 of SEQ ID NO: 21.

In another embodiment, a Clostridial toxin translocation domain comprises a TeNT translocation domain. In an aspect of this embodiment, a TeNT translocation domain comprises the translocation domains of SEQ ID NO: 22. In other aspects of this embodiment, a TeNT translocation domain comprises amino acids 468-881 of SEQ ID NO: 22. In another aspect of this embodiment, a TeNT translocation domain comprises a naturally occurring TeNT translocation domain variant, such as, e.g., an translocation domain from a TeNT isoform or an translocation domain from a TeNT subtype. In another aspect of this embodiment, a TeNT translocation domain comprises a naturally occurring TeNT translocation domain variant of SEQ ID NO: 22, such as, e.g., a TeNT isoform translocation domain or a TeNT subtype translocation domain. In another aspect of this embodiment, a TeNT translocation domain comprises amino acids 468-881 of a naturally occurring TeNT translocation domain variant of SEQ ID NO: 22, such as, e.g., a TeNT isoform translocation domain or a TeNT subtype translocation domain. In still another aspect of this embodiment, a TeNT translocation domain comprises a non-naturally occurring TeNT translocation domain variant, such as, e.g., a conservative TeNT translocation domain variant, a non-conservative TeNT translocation domain variant, an active TeNT translocation domain fragment, or any combination thereof. In still another aspect of this embodiment, a TeNT translocation domain comprises the translocation domain of a non-naturally occurring TeNT translocation domain variant of SEQ ID NO: 22, such as, e.g., a conservative TeNT translocation domain variant, a non-conservative TeNT translocation domain variant, an active TeNT translocation domain fragment, or any combination thereof. In still another aspect of this embodiment, a TeNT translocation domain comprises amino acids 468-881 of a non-naturally occurring TeNT translocation domain variant of SEQ ID NO: 22, such as, e.g., a conservative TeNT translocation domain variant, a non-conservative TeNT translocation domain variant, an active TeNT translocation domain fragment, or any combination thereof.

In other aspects of this embodiment, a TeNT translocation domain comprises a polypeptide having an amino acid identity of, e.g., at least 70%, at least 75%, at least 80%, at least 85%, at least 90%, or at least 95% to the translocation domain of SEQ ID NO: 22; or at most 70%, at most 75%, at most 80%, at most 85%, at most 90%, or at most 95% to the translocation domain of SEQ ID NO: 22. In yet other aspects of this embodiment, a TeNT translocation domain comprises a polypeptide having an amino acid identity of, e.g., at least 70%, at least 75%, at least 80%, at least 85%, at least 90%, or at least 95% to amino acids 468-881 of SEQ ID NO: 22; or at most 70%, at most 75%, at most 80%, at most 85%, at most 90%, or at most 95% to amino acids 468-881 of SEQ ID NO: 22.

In other aspects of this embodiment, a TeNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 22; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 22. In yet other aspects of this embodiment, a TeNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 468-881 of SEQ ID NO: 22; or at most 1, 2, 3, 4, 5, 6, 7, 8, 9, 10, 20, 30, 40, 50, or 100 non-contiguous amino acid deletions, additions, and/or substitutions relative to amino acids 468-881 of SEQ ID NO: 22. In still other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 22; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 22. In further other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 468-881 of SEQ ID NO: 22; or at most 1, 2, 3, 4, 5, 6, 7, 8, 9, 10, 20, 30, 40, 50, or 100 contiguous amino acid deletions, additions, and/or substitutions relative to amino acids 468-881 of SEQ ID NO: 22.

In another embodiment, a Clostridial toxin translocation domain comprises a BaNT translocation domain. In an aspect of this embodiment, a BaNT translocation domain comprises the translocation domains of SEQ ID NO: 23. In other aspects of this embodiment, a BaNT translocation domain comprises amino acids 436-857 of SEQ ID NO: 23. In another aspect of this embodiment, a BaNT translocation domain comprises a naturally occurring BaNT translocation domain variant, such as, e.g., an translocation domain from a BaNT isoform or an translocation domain from a BaNT subtype. In another aspect of this embodiment, a BaNT translocation domain comprises a naturally occurring BaNT translocation domain variant of SEQ ID NO: 23, such as, e.g., a BaNT isoform translocation domain or a BaNT subtype translocation domain. In another aspect of this embodiment, a BaNT translocation domain comprises amino acids 436-857 of a naturally occurring BaNT translocation domain variant of SEQ ID NO: 23, such as, e.g., a BaNT isoform translocation domain or a BaNT subtype translocation domain. In still another aspect of this embodiment, a BaNT translocation domain comprises a non-naturally occurring BaNT translocation domain variant, such as, e.g., a conservative BaNT translocation domain variant, a non-conservative BaNT translocation domain variant, an active BaNT translocation domain fragment, or any combination thereof. In still another aspect of this embodiment, a BaNT translocation domain comprises the translocation domain of a non-naturally occurring BaNT translocation domain variant of SEQ ID NO: 23, such as, e.g., a conservative BaNT translocation domain variant, a non-conservative BaNT translocation domain variant, an active BaNT translocation domain fragment, or any combination thereof. In still another aspect of this embodiment, a BaNT translocation domain comprises amino acids 436-857 of a non-naturally occurring BaNT translocation domain variant of SEQ ID NO: 23, such as, e.g., a conservative BaNT translocation domain variant, a non-conservative BaNT translocation domain variant, an active BaNT translocation domain fragment, or any combination thereof.

In other aspects of this embodiment, a BaNT translocation domain comprises a polypeptide having an amino acid identity of, e.g., at least 70%, at least 75%, at least 80%, at least 85%, at least 90%, or at least 95% to the translocation domain of SEQ ID NO: 23; or at most 70%, at most 75%, at most 80%, at most 85%, at most 90%, or at most 95% to the translocation domain of SEQ ID NO: 23. In yet other aspects of this embodiment, a BaNT translocation domain comprises a polypeptide having an amino acid identity of, e.g., at least 70%, at least 75%, at least 80%, at least 85%, at least 90%, or at least 95% to amino acids 436-857 of SEQ ID NO: 23; or at most 70%, at most 75%, at most 80%, at most 85%, at most 90%, or at most 95% to amino acids 436-857 of SEQ ID NO: 23.

In other aspects of this embodiment, a BaNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 23; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 23. In yet other aspects of this embodiment, a BaNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 436-857 of SEQ ID NO: 23; or at most 1, 2, 3, 4, 5, 6, 7, 8, 9, 10, 20, 30, 40, 50, or 100 non-contiguous amino acid deletions, additions, and/or substitutions relative to amino acids 436-857 of SEQ ID NO: 23. In still other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 23; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 23. In further other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 436-857 of SEQ ID NO: 23; or at most 1, 2, 3, 4, 5, 6, 7, 8, 9, 10, 20, 30, 40, 50, or 100 contiguous amino acid deletions, additions, and/or substitutions relative to amino acids 436-857 of SEQ ID NO: 23.

In another embodiment, a Clostridial toxin translocation domain comprises a BuNT translocation domain. In an aspect of this embodiment, a BuNT translocation domain comprises the translocation domains of SEQ ID NO: 24 or SEQ ID NO: 25. In other aspects of this embodiment, a BuNT translocation domain comprises amino acids 427-847 of SEQ ID NO: 24. In another aspect of this embodiment, a BuNT translocation domain comprises a naturally occurring BuNT translocation domain variant, such as, e.g., a translocation domain from a BuNT isoform or an translocation domain from a BuNT subtype. In another aspect of this embodiment, a BuNT translocation domain comprises a naturally occurring BuNT translocation domain variant of SEQ ID NO: 24 or SEQ ID NO: 25, such as, e.g., a BuNT isoform translocation domain or a BuNT subtype translocation domain. In another aspect of this embodiment, a BuNT translocation domain comprises amino acids 427-847 of a naturally occurring BuNT translocation domain variant of SEQ ID NO: 24, such as, e.g., a BuNT isoform translocation domain or a BuNT subtype translocation domain. In still another aspect of this embodiment, a BuNT translocation domain comprises a non-naturally occurring BuNT translocation domain variant, such as, e.g., a conservative BuNT translocation domain variant, a non-conservative BuNT translocation domain variant, an active BuNT translocation domain fragment, or any combination thereof. In still another aspect of this embodiment, a BuNT translocation domain comprises the translocation domain of a non-naturally occurring BuNT translocation domain variant of SEQ ID NO: 24 or SEQ ID NO: 25, such as, e.g., a conservative BuNT translocation domain variant, a non-conservative BuNT translocation domain variant, an active BuNT translocation domain fragment, or any combination thereof. In still another aspect of this embodiment, a BuNT translocation domain comprises amino acids 427-847 of a non-naturally occurring BuNT translocation domain variant of SEQ ID NO: 24, such as, e.g., a conservative BuNT translocation domain variant, a non-conservative BuNT translocation domain variant, an active BuNT translocation domain fragment, or any combination thereof.

In other aspects of this embodiment, a BuNT translocation domain comprises a polypeptide having an amino acid identity of, e.g., at least 70%, at least 75%, at least 80%, at least 85%, at least 90%, or at least 95% to the translocation domain of SEQ ID NO: 24 or SEQ ID NO: 25; or at most 70%, at most 75%, at most 80%, at most 85%, at most 90%, or at most 95% to the translocation domain of SEQ ID NO: 24 or SEQ ID NO: 25. In yet other aspects of this embodiment, a BuNT translocation domain comprises a polypeptide having an amino acid identity of, e.g., at least 70%, at least 75%, at least 80%, at least 85%, at least 90%, or at least 95% to amino acids 427-847 of SEQ ID NO: 24 or SEQ ID NO: 25; or at most 70%, at most 75%, at most 80%, at most 85%, at most 90%, or at most 95% to amino acids 427-847 of SEQ ID NO: 24 or SEQ ID NO: 25.

In other aspects of this embodiment, a BuNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 24 OR SEQ ID NO: 25. In yet other aspects of this embodiment, a BuNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 24 or SEQ ID NO: 25. In still other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 24 or SEQ ID NO: 25. In further other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 24 or SEQ ID NO: 25.

Aspects of the present specification provide, in part, a TVEMP comprising a targeting domain. As used herein, the term “targeting domain” is synonymous with “binding domain”, “ligand”, or “targeting moiety” and refers to an amino acid sequence region able to preferentially bind to a cell surface marker, like a receptor, characteristic of the target cell under physiological conditions. The cell surface marker may comprise a polypeptide, a glycoprotein, a lipoprotein, or may have structural characteristics of more than one of these. As used herein, the term “preferentially interacts” refers to a molecule capable of binding to its target cell surface marker under physiological conditions, or in vitro conditions substantially approximating physiological conditions, to a statistically significantly greater degree relative to other, non-target cell surface marker. With reference to a targeting domain disclosed herein, there is a discriminatory binding of the targeting domain to its cognate receptor relative to other receptors. Examples of binding domains are described in, e.g., Steward, L. E. et al., Modified Clostridial Toxins with Enhanced Translocation Capability and Enhanced Targeting Activity, U.S. patent application Ser. No. 11/776,043 (Jul. 11, 2007); Steward, L. E. et al., Modified Clostridial Toxins with Enhanced Translocation Capabilities and Altered Targeting Activity For Clostridial Toxin Target Cells, U.S. patent application Ser. No. 11/776,052 (Jul. 11, 2007); and Steward, L. E. et al., Modified Clostridial Toxins with Enhanced Translocation Capabilities and Altered Targeting Activity For Non-Clostridial Toxin Target Cells, U.S. patent application Ser. No. 11/776,075 (Jul. 11, 2007), each of which is incorporated by reference in its entirety.

In an embodiment, a binding domain that selectively binds a target receptor has a dissociation equilibrium constant (K_{D}) that is greater for the target receptor relative to a non-target receptor by, e.g., at least one-fold, at least two-fold, at least three-fold, at least four fold, at least five-fold, at least 10 fold, at least 50 fold, at least 100 fold, at least 1000, at least 10,000, or at least 100,000 fold.

An example of a targeting domain disclosed herein is an apelin targeting domain, an anthrax protective antigen (APA) targeting domain, or an angiogenic factor with G patch and FHA domain 1 (AGGF1) targeting domain. Apelin, APA, and AGGF1 targeting domains bind to transmembrane receptors present on the surface of endothelial cells associated with aberrant new blood vessel formation. For example, apelin receptors are expressed in endothelial cells participating in angiogenesis and neovasculogenesis associated with several disorders including ocular disorders. As another example, APA receptors like tumor endothelial marker 8 (TEM8) and capillary morphogeneisis gene 2 protein (CMG2) are expressed in endothelial cells undergoing angiogenesis in response to tumor progression, as well as endothelial cells participating in angiogenesis and neovasculogenesis associated with several disorders including ocular disorders. As yet another example, AGGF1 receptors are expressed in endothelial cells participating in angiogenesis and neovasculogenesis associated with several disorders including ocular disorders. As such, a TVEMP comprising an apelin targeting domain, an APA targeting domain, or an AGGF1 targeting domain would be effective in treating a disease or disorder associated with aberrant new blood vessel formation.

Thus, in an embodiment, a targeting domain comprises an apelin targeting domain, an APA targeting domain, or an AGGF1 targeting domain. In aspects of this embodiment, a targeting domain comprises an apelin targeting domain including SEQ ID NO: 82, SEQ ID NO: 83, SEQ ID NO: 84, SEQ ID NO: 85, SEQ ID NO: 86, or SEQ ID NO: 87; an APA targeting domain including SEQ ID NO: 88; or an AGGF1 targeting domain including SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98. In yet other aspects of this embodiment, a targeting domain comprises an apelin targeting domain including amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; an APA targeting domain including amino acids 30-764, amino acids 30-287, amino acids 30-196, amino acids 47-178, amino acids 204-628, amino acids 288-516, amino acids 517-624, amino acids 625-764 of SEQ ID NO: 89; or an AGGF1 targeting domain including amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98.

In other aspects of this embodiment, an apelin targeting domain, an APA targeting domain, or an AGGF1 targeting domain comprises a polypeptide having an amino acid identity of, e.g., at least 70%, at least 75%, at least 80%, at least 85%, at least 90% or at least 95% to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98; or at most 70%, at most 75%, at most 80%, at most 85%, at most 90% or at most 95% to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98. In yet other aspects of this embodiment, an apelin targeting domain, an APA targeting domain, or an AGGF1 targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98; or at most 1, 2, 3, 4, 5, 6, 7, 8, 9, 10, 15 or 20 non-contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98. In still other aspects of this embodiment an apelin targeting domain, an APA targeting domain, or an AGGF1 targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98; or at most 1, 2, 3, 4, 5, 6, 7, 8, 9, 10, 15 or 20 contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In other aspects of this embodiment, an apelin targeting domain, an APA targeting domain, or an AGGF1 targeting domain comprises a polypeptide having an amino acid identity of, e.g., at least 70%, at least 75%, at least 80%, at least 85%, at least 90% or at least 95% to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98; or at most 70%, at most 75%, at most 80%, at most 85%, at most 90% or at most 95% to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98. In yet other aspects of this embodiment, an apelin targeting domain, an APA targeting domain, or an AGGF1 targeting domain targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98; or at most 1, 2, 3, 4, 5, 6, 7, 8, 9, 10, 15 or 20 non-contiguous amino acid deletions, additions, and/or substitutions relative to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98. In still other aspects of this embodiment, an apelin targeting domain, an APA targeting domain, or an AGGF1 targeting domain targeting domain comprises a polypeptide having, e.g., at least 1, 2, 3, 4, 5, 6, 7, 8, 9, 10, 15 or 20 contiguous amino acid deletions, additions, and/or substitutions relative to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98; or at most 1, 2, 3, 4, 5, 6, 7, 8, 9, 10, 15 or 20 contiguous amino acid deletions, additions, and/or substitutions relative to amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; amino acids 30-764, amino acids 30-287, amino acids 30-196, amino acids 47-178, amino acids 204-628, amino acids 288-516, amino acids 517-624, amino acids 625-764 of SEQ ID NO: 89; or amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98.

An example of a targeting domain disclosed herein is an angiopoietin (Ang) targeting domain. Non-limiting examples of an angiopoietin targeting domain include an angiopoietin-1 targeting domain, an angiopoietin-2 targeting domain, an angiopoietin-3 targeting domain, and an angiopoietin-4 targeting domain. Angiopoietin targeting domains bind to transmembrane tyrosine kinase receptors present on the surface of endothelial cells associated with aberrant new blood vessel formation. For example, angiopoietin receptors like Tie-2 are expressed in endothelial cells participating in angiogenesis and neovasculogenesis associated with several disorders including ocular disorders. As such, a TVEMP comprising an angiopoietin targeting domain would be effective in treating a disease or disorder associated with aberrant new blood vessel formation.

Thus, in an embodiment, a targeting domain comprises an angiopoietin targeting domain. In aspects of this embodiment, an angiopoietin targeting domain comprises an angiopoietin-1 targeting domain, an angiopoietin-2 targeting domain, an angiopoietin-3 targeting domain, and an angiopoietin-4 targeting domain. In other aspects of this embodiment, an angiopoietin targeting domain comprises SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109. In yet other aspects of this embodiment, an angiopoietin targeting domain comprises amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109.

In other aspects of this embodiment, an angiopoietin targeting domain comprises a polypeptide having an amino acid identity of, e.g., at least 70%, at least 75%, at least 80%, at least 85%, at least 90% or at least 95% to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109; or at most 70%, at most 75%, at most 80%, at most 85%, at most 90% or at most 95% to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109. In yet other aspects of this embodiment, an angiopoietin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109; or at most 1, 2, 3, 4, 5, 6, 7, 8, 9, 10, 15 or 20 non-contiguous amino acid deletions, additions, and/or substitutions relative to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109. In still other aspects of this embodiment, an angiopoietin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109; or at most 1, 2, 3, 4, 5, 6, 7, 8, 9, 10, 15 or 20 contiguous amino acid deletions, additions, and/or substitutions relative to SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109.

In other aspects of this embodiment, an angiopoietin targeting domain comprises a polypeptide having an amino acid identity of, e.g., at least 70%, at least 75%, at least 80%, at least 85%, at least 90% or at least 95% to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109; or at most 70%, at most 75%, at most 80%, at most 85%, at most 90% or at most 95% to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109. In yet other aspects of this embodiment, an angiopoietin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109; or at most 1, 2, 3, 4, 5, 6, 7, 8, 9, 10, 15 or 20 non-contiguous amino acid deletions, additions, and/or substitutions relative to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109. In still other aspects of this embodiment, an angiopoietin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109; or at most 1, 2, 3, 4, 5, 6, 7, 8, 9, 10, 15 or 20 contiguous amino acid deletions, additions, and/or substitutions relative to amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109.

An example of a targeting domain disclosed herein is an angiostatin targeting domain or an angiogenin targeting domain. Angiostatin and angiogenin targeting domains bind to transmembrane receptors present on the surface of endothelial cells associated with aberrant new blood vessel formation. For example, angiostatin receptors like angiomotin, angiomotin-like 1/JEAP, angiomotin-like 2, and hepatocyte growth factor receptor c-met are expressed in endothelial cells undergoing angiogenesis in response to tumor progression, as well as endothelial cells participating in angiogenesis and neovasculogenesis associated with several disorders including cardiovascular disorders. As another example, angiogenin receptors like 170 kDa receptor are expressed in endothelial cells undergoing angiogenesis in response to tumor progression, as well as endothelial cells participating in angiogenesis and neovasculogenesis associated with several disorders including ocular disorders. As such, a TVEMP comprising an angiostatin targeting domain or an angiogenin targeting domain would be effective in treating a disease or disorder associated with aberrant new blood vessel formation.

Thus, in an embodiment, a targeting domain comprises an angiostatin targeting domain or an angiogenin targeting domain. In aspects of this embodiment, a targeting domain comprises an angiostatin targeting domain including SEQ ID NO: 110 or SEQ ID NO: 111; or an angiogenin targeting domain including SEQ ID NO: 112, SEQ ID NO: 113, SEQ ID NO: 114, SEQ ID NO: 115, or SEQ ID NO: 116. In yet other aspects of this embodiment, a targeting domain comprises an angiostatin targeting domain including amino acids 1-182, amino acids 1-262, amino acids 1-349, or amino acids 1-455 of SEQ ID NO: 110 or SEQ ID NO: 111; or an angiogenin targeting domain including amino acids 25-145 or amino acids 29-140 of SEQ ID NO: 112, SEQ ID NO: 113, SEQ ID NO: 114, SEQ ID NO: 115, or SEQ ID NO: 116.

In other aspects of this embodiment, an angiostatin targeting domain or an angiogenin targeting domain comprises a polypeptide having an amino acid identity of, e.g., at least 70%, at least 75%, at least 80%, at least 85%, at least 90% or at least 95% to SEQ ID NO: 110, SEQ ID NO: 111, SEQ ID NO: 112, SEQ ID NO: 113, SEQ ID NO: 114, SEQ ID NO: 115, or SEQ ID NO: 116; or at most 70%, at most 75%, at most 80%, at most 85%, at most 90% or at most 95% to SEQ ID NO: 110, SEQ ID NO: 111, SEQ ID NO: 112, SEQ ID NO: 113, SEQ ID NO: 114, SEQ ID NO: 115, or SEQ ID NO: 116. In yet other aspects of this embodiment, an angiostatin targeting domain or an angiogenin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 110, SEQ ID NO: 111, SEQ ID NO: 112, SEQ ID NO: 113, SEQ ID NO: 114, SEQ ID NO: 115, or SEQ ID NO: 116; or at most 1, 2, 3, 4, 5, 6, 7, 8, 9, 10, 15 or 20 non-contiguous amino acid deletions, additions, and/or substitutions relative to SEQ ID NO: 110, SEQ ID NO: 111, SEQ ID NO: 112, SEQ ID NO: 113, SEQ ID NO: 114, SEQ ID NO: 115, or SEQ ID NO: 116. In still other aspects of this embodiment an angiostatin targeting domain or an angiogenin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 110, SEQ ID NO: 111, SEQ ID NO: 112, SEQ ID NO: 113, SEQ ID NO: 114, SEQ ID NO: 115, or SEQ ID NO: 116; or at most 1, 2, 3, 4, 5, 6, 7, 8, 9, 10, 15 or 20 contiguous amino acid deletions, additions, and/or substitutions relative to SEQ ID NO: 110, SEQ ID NO: 111, SEQ ID NO: 112, SEQ ID NO: 113, SEQ ID NO: 114, SEQ ID NO: 115, or SEQ ID NO: 116.

In other aspects of this embodiment, an angiostatin targeting domain or an angiogenin targeting domain comprises a polypeptide having an amino acid identity of, e.g., at least 70%, at least 75%, at least 80%, at least 85%, at least 90% or at least 95% to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116; or at most 70%, at most 75%, at most 80%, at most 85%, at most 90% or at most 95% to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116. In yet other aspects of this embodiment, an angiostatin targeting domain or an angiogenin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116; or at most 1, 2, 3, 4, 5, 6, 7, 8, 9, 10, 15 or 20 non-contiguous amino acid deletions, additions, and/or substitutions relative to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116. In still other aspects of this embodiment, an angiostatin targeting domain or an angiogenin targeting domain targeting domain comprises a polypeptide having, e.g., at least 1, 2, 3, 4, 5, 6, 7, 8, 9, 10, 15 or 20 contiguous amino acid deletions, additions, and/or substitutions relative to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116; or at most 1, 2, 3, 4, 5, 6, 7, 8, 9, 10, 15 or 20 contiguous amino acid deletions, additions, and/or substitutions relative to amino acids 1-182, amino acids 1-262, amino acids 1-349, or amino acids 1-455 of SEQ ID NO: 110 or SEQ ID NO: 111; or amino acids 25-145 or amino acids 29-140 of SEQ ID NO: 112, SEQ ID NO: 113, SEQ ID NO: 114, SEQ ID NO: 115, or SEQ ID NO: 116.

An example of a targeting domain disclosed herein is an endothelin targeting domain or an endothelial cell-specific molecule (ECSM) targeting domain. Non-limiting examples of an endothelin targeting domain include an endothelin-1 targeting domain, an endothelin-2 targeting domain and an endothelin-3 targeting domain. Non-limiting examples of an ECSM targeting domain include an endothelial cell-specific molecule-1 (ECSM-1) targeting domain. Endothelin and ECSM targeting domains bind to transmembrane receptors present on the surface of endothelial cells associated with aberrant new blood vessel formation. For example, endothelin receptors are expressed in endothelial cells undergoing angiogenesis in response to tumor progression, as well as endothelial cells participating in angiogenesis and neovasculogenesis associated with several disorders including ocular disorders. As another example, ECSM receptors are expressed in endothelial participating in angiogenesis and neovasculogenesis associated with several disorders including ocular disorders. As such, a TVEMP comprising an endothelin targeting domain or an ECSM targeting domain would be effective in treating a disease or disorder associated with aberrant new blood vessel formation.

Thus, in an embodiment, a targeting domain comprises an endothelin targeting domain or an ECSM targeting domain. In aspects of this embodiment, an endothelin targeting domain is an endothelin-1 targeting domain, an endothelin-2 targeting domain and an endothelin-3 targeting domain. In other aspects of this embodiment, an ECSM targeting domain is an ECSM-1 targeting domain. In aspects of this embodiment, a targeting domain comprises an endothelin targeting domain including SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, or SEQ ID NO: 138; or an ECSM targeting domain including SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, or SEQ ID NO: 147. In yet other aspects of this embodiment, a targeting domain comprises an endothelin targeting domain including amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or an ECSM targeting domain including amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143.

In other aspects of this embodiment, an endothelin targeting domain or an ECSM targeting domain comprises a polypeptide having an amino acid identity of, e.g., at least 70%, at least 75%, at least 80%, at least 85%, at least 90% or at least 95% to SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, or SEQ ID NO: 147. In yet other aspects of this embodiment, an endothelin targeting domain or an ECSM targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, or SEQ ID NO: 147; or at most 1, 2, 3, 4, 5, 6, 7, 8, 9, 10, 15 or 20 non-contiguous amino acid deletions, additions, and/or substitutions relative to SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, or SEQ ID NO: 147. In still other aspects of this embodiment an endothelin targeting domain or an ECSM targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, or SEQ ID NO: 147.

In other aspects of this embodiment, an endothelin targeting domain or an ECSM targeting domain comprises a polypeptide having an amino acid identity of, e.g., at least 70%, at least 75%, at least 80%, at least 85%, at least 90% or at least 95% to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143; or at most 70%, at most 75%, at most 80%, at most 85%, at most 90% or at most 95% to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143. In yet other aspects of this embodiment, an endothelin targeting domain or an ECSM targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143; or at most 1, 2, 3, 4, 5, 6, 7, 8, 9, 10, 15 or 20 non-contiguous amino acid deletions, additions, and/or substitutions relative to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143. In still other aspects of this embodiment, an endothelin targeting domain or an ECSM targeting domain comprises a polypeptide having, e.g., at least 1, 2, 3, 4, 5, 6, 7, 8, 9, 10, 15 or 20 contiguous amino acid deletions, additions, and/or substitutions relative to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143; or at most 1, 2, 3, 4, 5, 6, 7, 8, 9, 10, 15 or 20 contiguous amino acid deletions, additions, and/or substitutions relative to amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143.

An example of a targeting domain disclosed herein is an ephrin targeting domain. Non-limiting examples of an ephrin targeting domain include an ephrin-A1 targeting domain and an ephrin-B2 targeting domain. Ephrin targeting domains bind to transmembrane receptors present on the surface of endothelial cells associated with aberrant new blood vessel formation. For example, ephrin receptors like ephrin-A2 receptor, ephrin-A3 receptor and ephrin-A4 receptor, ephrin-B2 receptor and ephrin-B4 receptor are expressed in endothelial cells undergoing angiogenesis in response to tumor progression, as well as endothelial cells participating in angiogenesis and neovasculogenesis associated with several disorders including ocular disorders. As such, a TVEMP comprising an ephrin targeting domain would be effective in treating a disease or disorder associated with aberrant new blood vessel formation.

Thus, in an embodiment, a targeting domain comprises an ephrin targeting domain. In aspects of this embodiment, an ephrin targeting domain comprises an ephrin-A1 targeting domain and an ephrin-B2 targeting domain. In other aspects of this embodiment, an ephrin targeting domain comprises SEQ ID NO: 148, SEQ ID NO: 149, SEQ ID NO: 150, SEQ ID NO: 151, SEQ ID NO: 152, SEQ ID NO: 153, or SEQ ID NO: 154. In yet other aspects of this embodiment, an ephrin targeting domain comprises amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154.

In other aspects of this embodiment, an ephrin targeting domain comprises a polypeptide having an amino acid identity of, e.g., at least 70%, at least 75%, at least 80%, at least 85%, at least 90% or at least 95% to SEQ ID NO: 148, SEQ ID NO: 149, SEQ ID NO: 150, SEQ ID NO: 151, SEQ ID NO: 152, SEQ ID NO: 153, or SEQ ID NO: 154; or at most 70%, at most 75%, at most 80%, at most 85%, at most 90% or at most 95% to SEQ ID NO: 148, SEQ ID NO: 149, SEQ ID NO: 150, SEQ ID NO: 151, SEQ ID NO: 152, SEQ ID NO: 153, or SEQ ID NO: 154. In yet other aspects of this embodiment, an ephrin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 148, SEQ ID NO: 149, SEQ ID NO: 150, SEQ ID NO: 151, SEQ ID NO: 152, SEQ ID NO: 153, or SEQ ID NO: 154; or at most 1, 2, 3, 4, 5, 6, 7, 8, 9, 10, 15 or 20 non-contiguous amino acid deletions, additions, and/or substitutions relative to SEQ ID NO: 148, SEQ ID NO: 149, SEQ ID NO: 150, SEQ ID NO: 151, SEQ ID NO: 152, SEQ ID NO: 153, or SEQ ID NO: 154. In still other aspects of this embodiment, an ephrin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 148, SEQ ID NO: 149, SEQ ID NO: 150, SEQ ID NO: 151, SEQ ID NO: 152, SEQ ID NO: 153, or SEQ ID NO: 154; or at most 1, 2, 3, 4, 5, 6, 7, 8, 9, 10, 15 or 20 contiguous amino acid deletions, additions, and/or substitutions relative to SEQ ID NO: 148, SEQ ID NO: 149, SEQ ID NO: 150, SEQ ID NO: 151, SEQ ID NO: 152, SEQ ID NO: 153, or SEQ ID NO: 154.

In other aspects of this embodiment, an ephrin targeting domain comprises a polypeptide having an amino acid identity of, e.g., at least 70%, at least 75%, at least 80%, at least 85%, at least 90% or at least 95% to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154; or at most 70%, at most 75%, at most 80%, at most 85%, at most 90% or at most 95% to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154. In yet other aspects of this embodiment, an ephrin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154; or at most 1, 2, 3, 4, 5, 6, 7, 8, 9, 10, 15 or 20 non-contiguous amino acid deletions, additions, and/or substitutions relative to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154. In still other aspects of this embodiment, an ephrin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154; or at most 1, 2, 3, 4, 5, 6, 7, 8, 9, 10, 15 or 20 contiguous amino acid deletions, additions, and/or substitutions relative to amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154.

An example of a targeting domain disclosed herein is an erythropoietin targeting domain. Erythropoietin targeting domains bind to transmembrane receptors present on the surface of endothelial cells associated with aberrant new blood vessel formation. For example, erythropoietin receptors are expressed in endothelial cells participating in angiogenesis and neovasculogenesis associated with several disorders including ocular disorders. As such, a TVEMP comprising an erythropoietin targeting domain would be effective in treating a disease or disorder associated with aberrant new blood vessel formation.

Thus, in an embodiment, a targeting domain comprises an erythropoietin targeting domain. In aspects of this embodiment, an erythropoietin targeting domain comprises SEQ ID NO: 155, SEQ ID NO: 156, or SEQ ID NO: 157. In yet other aspects of this embodiment, an erythropoietin targeting domain comprises amino acids 33-193 of SEQ ID NO: 155; or amino acids 26-192 of SEQ ID NO: 156 or SEQ ID NO: 157.

In other aspects of this embodiment, an erythropoietin targeting domain comprises a polypeptide having an amino acid identity of, e.g., at least 70%, at least 75%, at least 80%, at least 85%, at least 90% or at least 95% to SEQ ID NO: 155, SEQ ID NO: 156, or SEQ ID NO: 157; or at most 70%, at most 75%, at most 80%, at most 85%, at most 90% or at most 95% to SEQ ID NO: 155, SEQ ID NO: 156, or SEQ ID NO: 157. In yet other aspects of this embodiment, an erythropoietin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 155, SEQ ID NO: 156, or SEQ ID NO: 157; or at most 1, 2, 3, 4, 5, 6, 7, 8, 9, 10, 15 or 20 non-contiguous amino acid deletions, additions, and/or substitutions relative to SEQ ID NO: 155, SEQ ID NO: 156, or SEQ ID NO: 157. In still other aspects of this embodiment, an erythropoietin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 155, SEQ ID NO: 156, or SEQ ID NO: 157; or at most 1, 2, 3, 4, 5, 6, 7, 8, 9, 10, 15 or 20 contiguous amino acid deletions, additions, and/or substitutions relative to SEQ ID NO: 155, SEQ ID NO: 156, or SEQ ID NO: 157.

In other aspects of this embodiment, an erythropoietin targeting domain comprises a polypeptide having an amino acid identity of, e.g., at least 70%, at least 75%, at least 80%, at least 85%, at least 90% or at least 95% to amino acids 33-193 of SEQ ID NO: 155; or amino acids 26-192 of SEQ ID NO: 156 or SEQ ID NO: 157; or at most 70%, at most 75%, at most 80%, at most 85%, at most 90% or at most 95% to amino acids 33-193 of SEQ ID NO: 155; or amino acids 26-192 of SEQ ID NO: 156 or SEQ ID NO: 157. In yet other aspects of this embodiment, an erythropoietin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 33-193 of SEQ ID NO: 155; or amino acids 26-192 of SEQ ID NO: 156 or SEQ ID NO: 157; or at most 1, 2, 3, 4, 5, 6, 7, 8, 9, 10, 15 or 20 non-contiguous amino acid deletions, additions, and/or substitutions relative to amino acids 33-193 of SEQ ID NO: 155; or amino acids 26-192 of SEQ ID NO: 156 or SEQ ID NO: 157. In still other aspects of this embodiment, an erythropoietin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 33-193 of SEQ ID NO: 155; or amino acids 26-192 of SEQ ID NO: 156 or SEQ ID NO: 157; or at most 1, 2, 3, 4, 5, 6, 7, 8, 9, 10, 15 or 20 contiguous amino acid deletions, additions, and/or substitutions relative to amino acids 33-193 of SEQ ID NO: 155; or amino acids 26-192 of SEQ ID NO: 156 or SEQ ID NO: 157.

An example of a targeting domain disclosed herein is a hepatocyte growth factor (HGF) targeting domain. Non-limiting examples of a HGF targeting domain include a HGF targeting domain, a NK1 targeting domain, a NK2 targeting domain, a NK3 targeting domain, and a NK4 targeting domain. HGF targeting domains bind to transmembrane receptors present on the surface of endothelial cells associated with aberrant new blood vessel formation. For example, HGF receptors like HGF c-met are expressed in endothelial cells undergoing angiogenesis in response to tumor progression, as well as in endothelial cells participating in angiogenesis and neovasculogenesis associated with several disorders including ocular disorders and skin disorders. As such, a TVEMP comprising a HGF targeting domain would be effective in treating a disease or disorder associated with aberrant new blood vessel formation.

Thus, in an embodiment, a targeting domain comprises a HGF targeting domain. In aspects of this embodiment, a HGF targeting domain comprises a HGF targeting domain, a NK1 targeting domain, a NK2 targeting domain, a NK3 targeting domain, and a NK4 targeting domain. In other aspects of this embodiment, a HGF targeting domain comprises SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172.

In other aspects of this embodiment, a HGF targeting domain comprises a polypeptide having an amino acid identity of, e.g., at least 70%, at least 75%, at least 80%, at least 85%, at least 90% or at least 95% to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172; or at most 70%, at most 75%, at most 80%, at most 85%, at most 90% or at most 95% to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 17257. In yet other aspects of this embodiment, a HGF targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172; or at most 1, 2, 3, 4, 5, 6, 7, 8, 9, 10, 15 or 20 non-contiguous amino acid deletions, additions, and/or substitutions relative to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172. In still other aspects of this embodiment, a HGF targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172; or at most 1, 2, 3, 4, 5, 6, 7, 8, 9, 10, 15 or 20 contiguous amino acid deletions, additions, and/or substitutions relative to SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172.

An example of a targeting domain disclosed herein is a netrin targeting domain. Non-limiting examples of a netrin targeting domain include a netrin-1 targeting domain, a netrin-2 targeting domain, a netrin-3 targeting domain, a netrin-4 targeting domain, a netrin-5 targeting domain, a netrin-G1 targeting domain, and a netrin-G2 targeting domain. Netrin targeting domains bind to transmembrane receptors present on the surface of endothelial cells associated with aberrant new blood vessel formation. For example, netrin receptors are expressed in endothelial cells undergoing angiogenesis in response to tumor progression, as well as in endothelial cells participating in angiogenesis and neovasculogenesis associated with several disorders including ocular disorders. As such, a TVEMP comprising a netrin targeting domain would be effective in treating a disease or disorder associated with aberrant new blood vessel formation.

173-216

Thus, in an embodiment, a targeting domain comprises a netrin targeting domain. In aspects of this embodiment, a netrin targeting domain is a netrin-1 targeting domain, a netrin-2 targeting domain, a netrin-3 targeting domain, a netrin-4 targeting domain, a netrin-5 targeting domain, a netrin-G1 targeting domain, and a netrin-G2 targeting domain. In aspects of this embodiment, a netrin targeting domain comprises SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216.

In yet other aspects of this embodiment, a netrin targeting domain comprises amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216.

In other aspects of this embodiment, a netrin targeting domain comprises a polypeptide having an amino acid identity of, e.g., at least 70%, at least 75%, at least 80%, at least 85%, at least 90% or at least 95% to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216; or at most 70%, at most 75%, at most 80%, at most 85%, at most 90% or at most 95% to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216. In yet other aspects of this embodiment, a netrin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216; or at most 1, 2, 3, 4, 5, 6, 7, 8, 9, 10, 15 or 20 non-contiguous amino acid deletions, additions, and/or substitutions relative to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216. In still other aspects of this embodiment, a netrin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216; or at most 1, 2, 3, 4, 5, 6, 7, 8, 9, 10, 15 or 20 contiguous amino acid deletions, additions, and/or substitutions relative to SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, or SEQ ID NO: 216.

In other aspects of this embodiment, a netrin targeting domain comprises a polypeptide having an amino acid identity of, e.g., at least 70%, at least 75%, at least 80%, at least 85%, at least 90% or at least 95% to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216; or at most 70%, at most 75%, at most 80%, at most 85%, at most 90% or at most 95% to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216.

In yet other aspects of this embodiment, a netrin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216; or at most 1, 2, 3, 4, 5, 6, 7, 8, 9, 10, 15 or 20 non-contiguous amino acid deletions, additions, and/or substitutions relative to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216.

In still other aspects of this embodiment, a netrin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216; or at most 1, 2, 3, 4, 5, 6, 7, 8, 9, 10, 15 or 20 contiguous amino acid deletions, additions, and/or substitutions relative to amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216.

An example of a targeting domain disclosed herein is a Delta-like (DII) targeting domain or a jagged targeting domain. Non-limiting examples of a DII targeting domain include a Delta-like-1 targeting domain, a Delta-like-2 targeting domain, and a Delta-like-3 targeting domain. Non-limiting examples of a jagged targeting domain include a jagged-1 targeting domain and a jagged-2 targeting domain. DII and jagged targeting domains bind to transmembrane receptors present on the surface of endothelial cells associated with aberrant new blood vessel formation. For example, DII and jagged receptors like Notch-1, Notch-2, Notch-3 and Notch-4 are expressed in endothelial cells undergoing angiogenesis in response to tumor progression. As such, a TVEMP comprising a DII targeting domain or a jagged targeting domain would be effective in treating a disease or disorder associated with aberrant new blood vessel formation.

Thus, in an embodiment, a targeting domain comprises a DII targeting domain or a jagged targeting domain. In aspects of this embodiment a DII targeting domain is a Delta-like-1 targeting domain, a Delta-like-2 targeting domain, and a Delta-like-3 targeting domain. In other aspects of this embodiment a jagged targeting domain is a jagged-1 targeting domain and a jagged-2 targeting domain. In aspects of this embodiment, a targeting domain comprises a DII targeting domain including SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, or SEQ ID NO: 229; a jagged targeting domain including SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237. In yet other aspects of this embodiment, a targeting domain comprises a DII targeting domain including amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; or a jagged targeting domain including amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236.

In other aspects of this embodiment, a DII targeting domain or a jagged targeting domain comprises a polypeptide having an amino acid identity of, e.g., at least 70%, at least 75%, at least 80%, at least 85%, at least 90% or at least 95% to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237; or at most 70%, at most 75%, at most 80%, at most 85%, at most 90% or at most 95% to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237. In yet other aspects of this embodiment, a DII targeting domain or a jagged targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237; or at most 1, 2, 3, 4, 5, 6, 7, 8, 9, 10, 15 or 20 non-contiguous amino acid deletions, additions, and/or substitutions relative to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237. In still other aspects of this embodiment a DII targeting domain or a jagged targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237; or at most 1, 2, 3, 4, 5, 6, 7, 8, 9, 10, 15 or 20 contiguous amino acid deletions, additions, and/or substitutions relative to SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, or SEQ ID NO: 237.

In other aspects of this embodiment, a DII targeting domain or a jagged targeting domain comprises a polypeptide having an amino acid identity of, e.g., at least 70%, at least 75%, at least 80%, at least 85%, at least 90% or at least 95% to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236; or at most 70%, at most 75%, at most 80%, at most 85%, at most 90% or at most 95% to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236. In yet other aspects of this embodiment, a DII targeting domain or a jagged targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236; or at most 1, 2, 3, 4, 5, 6, 7, 8, 9, 10, 15 or 20 non-contiguous amino acid deletions, additions, and/or substitutions relative to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236. In still other aspects of this embodiment, a DII targeting domain or a jagged targeting domain comprises a polypeptide having, e.g., at least 1, 2, 3, 4, 5, 6, 7, 8, 9, 10, 15 or 20 contiguous amino acid deletions, additions, and/or substitutions relative to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236; or at most 1, 2, 3, 4, 5, 6, 7, 8, 9, 10, 15 or 20 contiguous amino acid deletions, additions, and/or substitutions relative to amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236.

An example of a targeting domain disclosed herein is a semaphorin (Sema) targeting domain. Non-limiting examples of a Sema targeting domain include a semaphoring-3A targeting domain, a semaphoring-3F targeting domain, a semaphoring-4D targeting domain, and a semaphoring-5A targeting domain. Sema targeting domains bind to transmembrane receptors present on the surface of endothelial cells associated with aberrant new blood vessel formation. For example, Sema receptors like neurophillin-1, neurophin-2, plexin-A1, plexin-A2, plexin-B and plexin-B3 are expressed in endothelial cells undergoing angiogenesis in response to tumor progression. As such, a TVEMP comprising a Sema targeting domain would be effective in treating a disease or disorder associated with aberrant new blood vessel formation.

Thus, in an embodiment, a targeting domain comprises a Sema targeting domain. In aspects of this embodiment, a Sema targeting domain comprises a semaphoring-3A targeting domain, a semaphoring-3F targeting domain, a semaphoring-4D targeting domain, and a semaphoring-5A targeting domain. In other aspects of this embodiment, a Sema targeting domain comprises SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246. In yet other aspects of this embodiment, a Sema targeting domain comprises amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246.

In other aspects of this embodiment, a Sema targeting domain comprises a polypeptide having an amino acid identity of, e.g., at least 70%, at least 75%, at least 80%, at least 85%, at least 90% or at least 95% to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246; or at most 70%, at most 75%, at most 80%, at most 85%, at most 90% or at most 95% to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246. In yet other aspects of this embodiment, a Sema targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246; or at most 1, 2, 3, 4, 5, 6, 7, 8, 9, 10, 15 or 20 non-contiguous amino acid deletions, additions, and/or substitutions relative to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246. In still other aspects of this embodiment, a Sema targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246; or at most 1, 2, 3, 4, 5, 6, 7, 8, 9, 10, 15 or 20 contiguous amino acid deletions, additions, and/or substitutions relative to SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, or SEQ ID NO: 246.

In other aspects of this embodiment, a Sema targeting domain comprises a polypeptide having an amino acid identity of, e.g., at least 70%, at least 75%, at least 80%, at least 85%, at least 90% or at least 95% to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246; or at most 70%, at most 75%, at most 80%, at most 85%, at most 90% or at most 95% to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246. In yet other aspects of this embodiment, a Sema targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246; or at most 1, 2, 3, 4, 5, 6, 7, 8, 9, 10, 15 or 20 non-contiguous amino acid deletions, additions, and/or substitutions relative to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246. In still other aspects of this embodiment, a Sema targeting domain comprises a polypeptide having, e.g., at least 1, 2, 3, 4, 5, 6, 7, 8, 9, 10, 15 or 20 contiguous amino acid deletions, additions, and/or substitutions relative to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246; or at most 1, 2, 3, 4, 5, 6, 7, 8, 9, 10, 15 or 20 contiguous amino acid deletions, additions, and/or substitutions relative to amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246.

An example of a targeting domain disclosed herein is a thrombospondin targeting domain or a brain-specific angiogenesis inhibitor (BSAI) targeting domain. Non-limiting examples of a thrombospondin targeting domain include a thrombospondin-1 targeting domain and a thrombospondin-2 targeting domain. Non-limiting examples of a BSAI targeting domain include a BSAI-1 targeting domain and a BSAI-2 targeting domain. Thrombospondin and BSAI targeting domains bind to transmembrane receptors present on the surface of endothelial cells associated with aberrant new blood vessel formation. For example, thrombospondin and BSAI receptors like CD36 are expressed in endothelial cells undergoing angiogenesis in response to tumor progression. As such, a TVEMP comprising a thrombospondin targeting domain or a BSAI targeting domain would be effective in treating a disease or disorder associated with aberrant new blood vessel formation.

Thus, in an embodiment, a targeting domain comprises a thrombospondin targeting domain or a BSAI targeting domain. In aspects of this embodiment, a thrombospondin targeting domain is a thrombospondin-1 targeting domain or a thrombospondin-2 targeting domain. In other aspects of this embodiment, a BSAI targeting domain is a vaculostatin targeting domain. In aspects of this embodiment, a targeting domain comprises a thrombospondin targeting domain including SEQ ID NO: 247, SEQ ID NO: 248, SEQ ID NO: 249, or SEQ ID NO: 250; or a BSAI targeting domain including SEQ ID NO: 217 or SEQ ID NO: 218. In yet other aspects of this embodiment, a targeting domain comprises a thrombospondin targeting domain including amino acids 19-1170 of SEQ ID NO: 247, SEQ ID NO: 248, SEQ ID NO: 249, or SEQ ID NO: 250; or a BSAI targeting domain including amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218.

In other aspects of this embodiment, a thrombospondin targeting domain or a BSAI targeting domain comprises a polypeptide having an amino acid identity of, e.g., at least 70%, at least 75%, at least 80%, at least 85%, at least 90% or at least 95% to SEQ ID NO: 247, SEQ ID NO: 248, SEQ ID NO: 249, SEQ ID NO: 250, SEQ ID NO: 217 or SEQ ID NO: 218; or at most 70%, at most 75%, at most 80%, at most 85%, at most 90% or at most 95% to SEQ ID NO: 247, SEQ ID NO: 248, SEQ ID NO: 249, SEQ ID NO: 250, SEQ ID NO: 217 or SEQ ID NO: 218. In yet other aspects of this embodiment, a thrombospondin targeting domain or a BSAI targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 247, SEQ ID NO: 248, SEQ ID NO: 249, SEQ ID NO: 250, SEQ ID NO: 217 or SEQ ID NO: 218; or at most 1, 2, 3, 4, 5, 6, 7, 8, 9, 10, 15 or 20 non-contiguous amino acid deletions, additions, and/or substitutions relative to SEQ ID NO: 247, SEQ ID NO: 248, SEQ ID NO: 249, SEQ ID NO: 250, SEQ ID NO: 217 or SEQ ID NO: 218. In still other aspects of this embodiment a thrombospondin targeting domain or a BSAI targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 247, SEQ ID NO: 248, SEQ ID NO: 249, SEQ ID NO: 250, SEQ ID NO: 217 or SEQ ID NO: 218; or at most 1, 2, 3, 4, 5, 6, 7, 8, 9, 10, 15 or 20 contiguous amino acid deletions, additions, and/or substitutions relative to SEQ ID NO: 247, SEQ ID NO: 248, SEQ ID NO: 249, SEQ ID NO: 250, SEQ ID NO: 217 or SEQ ID NO: 218.

In other aspects of this embodiment, a thrombospondin targeting domain or a BSAI targeting domain comprises a polypeptide having an amino acid identity of, e.g., at least 70%, at least 75%, at least 80%, at least 85%, at least 90% or at least 95% to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218; or at most 70%, at most 75%, at most 80%, at most 85%, at most 90% or at most 95% to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218. In yet other aspects of this embodiment, a thrombospondin targeting domain or a BSAI targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218; or at most 1, 2, 3, 4, 5, 6, 7, 8, 9, 10, 15 or 20 non-contiguous amino acid deletions, additions, and/or substitutions relative to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218. In still other aspects of this embodiment, a thrombospondin targeting domain or a BSAI targeting domain comprises a polypeptide having, e.g., at least 1, 2, 3, 4, 5, 6, 7, 8, 9, 10, 15 or 20 contiguous amino acid deletions, additions, and/or substitutions relative to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218; or at most 1, 2, 3, 4, 5, 6, 7, 8, 9, 10, 15 or 20 contiguous amino acid deletions, additions, and/or substitutions relative to amino acids 19-1170 of SEQ ID NO: 247, SEQ ID NO: 248, SEQ ID NO: 249, SEQ ID NO: 250, amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218.

An example of a targeting domain disclosed herein is a chondromodulin targeting domain or a 16 kDa prolactin fragment targeting domain. Chondromodulin and 16 kDa prolactin fragment targeting domains bind to transmembrane receptors present on the surface of endothelial cells associated with aberrant new blood vessel formation. For example, chondromodulin receptors are expressed in endothelial cells undergoing angiogenesis in response to tumor progression. As another example, 16 kDa prolactin fragment receptors are expressed in endothelial cells undergoing angiogenesis in response to tumor progression, as well as endothelial cells participating in angiogenesis and neovasculogenesis associated with several disorders including ocular disorders. As such, a TVEMP comprising a chondromodulin targeting domain or a 16 kDa prolactin fragment targeting domain would be effective in treating a disease or disorder associated with aberrant new blood vessel formation.

Thus, in an embodiment, a targeting domain comprises a chondromodulin targeting domain or a 16 kDa prolactin fragment targeting domain. In aspects of this embodiment, a targeting domain comprises a chondromodulin targeting domain including SEQ ID NO: 251, SEQ ID NO: 252, or SEQ ID NO: 253; or a 16 kDa prolactin fragment targeting domain including SEQ ID NO: 254. In yet other aspects of this embodiment, a targeting domain comprises a chondromodulin targeting domain including amino acids 214-334 of SEQ ID NO: 251 or SEQ ID NO: 253, or amino acids 215-335 of SEQ ID NO: 252.

In other aspects of this embodiment, a chondromodulin targeting domain or a 16 kDa prolactin fragment targeting domain comprises a polypeptide having an amino acid identity of, e.g., at least 70%, at least 75%, at least 80%, at least 85%, at least 90% or at least 95% to SEQ ID NO: 251, SEQ ID NO: 252, SEQ ID NO: 253, or SEQ ID NO: 254; or at most 70%, at most 75%, at most 80%, at most 85%, at most 90% or at most 95% to SEQ ID NO: 251, SEQ ID NO: 252, SEQ ID NO: 253, or SEQ ID NO: 254. In yet other aspects of this embodiment, a chondromodulin targeting domain or a 16 kDa prolactin fragment targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 251, SEQ ID NO: 252, SEQ ID NO: 253, or SEQ ID NO: 254; or at most 1, 2, 3, 4, 5, 6, 7, 8, 9, 10, 15 or 20 non-contiguous amino acid deletions, additions, and/or substitutions relative to SEQ ID NO: 251, SEQ ID NO: 252, SEQ ID NO: 253, or SEQ ID NO: 254. In still other aspects of this embodiment a chondromodulin targeting domain or a 16 kDa prolactin fragment targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 251, SEQ ID NO: 252, SEQ ID NO: 253, or SEQ ID NO: 254; or at most 1, 2, 3, 4, 5, 6, 7, 8, 9, 10, 15 or 20 contiguous amino acid deletions, additions, and/or substitutions relative to SEQ ID NO: 251, SEQ ID NO: 252, SEQ ID NO: 253, or SEQ ID NO: 254.

In other aspects of this embodiment, a chondromodulin targeting domain comprises a polypeptide having an amino acid identity of, e.g., at least 70%, at least 75%, at least 80%, at least 85%, at least 90% or at least 95% to amino acids 214-334 of SEQ ID NO: 251 or SEQ ID NO: 253, or amino acids 215-335 of SEQ ID NO: 252; or at most 70%, at most 75%, at most 80%, at most 85%, at most 90% or at most 95% to amino acids 214-334 of SEQ ID NO: 251 or SEQ ID NO: 253, or amino acids 215-335 of SEQ ID NO: 252. In yet other aspects of this embodiment, a chondromodulin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 214-334 of SEQ ID NO: 251 or SEQ ID NO: 253, or amino acids 215-335 of SEQ ID NO: 252; or at most 1, 2, 3, 4, 5, 6, 7, 8, 9, 10, 15 or 20 non-contiguous amino acid deletions, additions, and/or substitutions relative to amino acids 214-334 of SEQ ID NO: 251 or SEQ ID NO: 253, or amino acids 215-335 of SEQ ID NO: 252. In still other aspects of this embodiment, a chondromodulin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 214-334 of SEQ ID NO: 251 or SEQ ID NO: 253, or amino acids 215-335 of SEQ ID NO: 252; or at most 1, 2, 3, 4, 5, 6, 7, 8, 9, 10, 15 or 20 contiguous amino acid deletions, additions, and/or substitutions relative to amino acids 214-334 of SEQ ID NO: 251 or SEQ ID NO: 253, or amino acids 215-335 of SEQ ID NO: 252.

An example of a targeting domain disclosed herein is a granulocyte macrophage-colony stimulating factor (GM-CSF) targeting domain. GM-CSF targeting domains bind to transmembrane receptors present on the surface of endothelial cells associated with aberrant new blood vessel formation. For example, GM-CSF receptors are expressed in endothelial cells undergoing angiogenesis in response to tumor progression. As such, a TVEMP comprising a GM-CSF targeting domain would be effective in treating a disease or disorder associated with aberrant new blood vessel formation.

Thus, in an embodiment, a targeting domain comprises a GM-CSF targeting domain. In aspects of this embodiment, a GM-CSF targeting domain comprises SEQ ID NO: 255, SEQ ID NO: 256, SEQ ID NO: 257, SEQ ID NO: 258, SEQ ID NO: 259, SEQ ID NO: 260, or SEQ ID NO: 261. In yet other aspects of this embodiment, a GM-CSF targeting domain comprises amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259.

In other aspects of this embodiment, a GM-CSF targeting domain comprises a polypeptide having an amino acid identity of, e.g., at least 70%, at least 75%, at least 80%, at least 85%, at least 90% or at least 95% to SEQ ID NO: 255, SEQ ID NO: 256, SEQ ID NO: 257, SEQ ID NO: 258, SEQ ID NO: 259, SEQ ID NO: 260, or SEQ ID NO: 261; or at most 70%, at most 75%, at most 80%, at most 85%, at most 90% or at most 95% to SEQ ID NO: 255, SEQ ID NO: 256, SEQ ID NO: 257, SEQ ID NO: 258, SEQ ID NO: 259, SEQ ID NO: 260, or SEQ ID NO: 261. In yet other aspects of this embodiment, a GM-CSF targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 255, SEQ ID NO: 256, SEQ ID NO: 257, SEQ ID NO: 258, SEQ ID NO: 259, SEQ ID NO: 260, or SEQ ID NO: 261; or at most 1, 2, 3, 4, 5, 6, 7, 8, 9, 10, 15 or 20 non-contiguous amino acid deletions, additions, and/or substitutions relative to SEQ ID NO: 255, SEQ ID NO: 256, SEQ ID NO: 257, SEQ ID NO: 258, SEQ ID NO: 259, SEQ ID NO: 260, or SEQ ID NO: 261. In still other aspects of this embodiment, a GM-CSF targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 255, SEQ ID NO: 256, SEQ ID NO: 257, SEQ ID NO: 258, SEQ ID NO: 259, SEQ ID NO: 260, or SEQ ID NO: 261; or at most 1, 2, 3, 4, 5, 6, 7, 8, 9, 10, 15 or 20 contiguous amino acid deletions, additions, and/or substitutions relative to SEQ ID NO: 255, SEQ ID NO: 256, SEQ ID NO: 257, SEQ ID NO: 258, SEQ ID NO: 259, SEQ ID NO: 260, or SEQ ID NO: 261.

In other aspects of this embodiment, a GM-CSF targeting domain comprises a polypeptide having an amino acid identity of, e.g., at least 70%, at least 75%, at least 80%, at least 85%, at least 90% or at least 95% to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259; or at most 70%, at most 75%, at most 80%, at most 85%, at most 90% or at most 95% to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259. In yet other aspects of this embodiment, a GM-CSF targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259; or at most 1, 2, 3, 4, 5, 6, 7, 8, 9, 10, 15 or 20 non-contiguous amino acid deletions, additions, and/or substitutions relative to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259. In still other aspects of this embodiment, a GM-CSF targeting domain comprises a polypeptide having, e.g., at least 1, 2, 3, 4, 5, 6, 7, 8, 9, 10, 15 or 20 contiguous amino acid deletions, additions, and/or substitutions relative to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259; or at most 1, 2, 3, 4, 5, 6, 7, 8, 9, 10, 15 or 20 contiguous amino acid deletions, additions, and/or substitutions relative to amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259.

An example of a targeting domain disclosed herein is an Interferon-α (IFN-α) targeting domain. IFN-α targeting domains bind to transmembrane receptors present on the surface of endothelial cells associated with aberrant new blood vessel formation. For example, IFN-α receptors like IFNAR1 and IFNAR2 are expressed in endothelial cells undergoing angiogenesis in response to tumor progression. As such, a TVEMP comprising an IFN-α targeting domain would be effective in treating a disease or disorder associated with aberrant new blood vessel formation.

Thus, in an embodiment, a targeting domain comprises an IFN-α targeting domain. In aspects of this embodiment, an IFN-α targeting domain comprises SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270. In yet other aspects of this embodiment, an IFN-α targeting domain comprises amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270.

In other aspects of this embodiment, an IFN-α targeting domain comprises a polypeptide having an amino acid identity of, e.g., at least 70%, at least 75%, at least 80%, at least 85%, at least 90% or at least 95% to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 70%, at most 75%, at most 80%, at most 85%, at most 90% or at most 95% to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270. In yet other aspects of this embodiment, an IFN-α targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 1, 2, 3, 4, 5, 6, 7, 8, 9, 10, 15 or 20 non-contiguous amino acid deletions, additions, and/or substitutions relative to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270. In still other aspects of this embodiment, an IFN-α targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 1, 2, 3, 4, 5, 6, 7, 8, 9, 10, 15 or 20 contiguous amino acid deletions, additions, and/or substitutions relative to SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270.

In other aspects of this embodiment, an IFN-α targeting domain comprises a polypeptide having an amino acid identity of, e.g., at least 70%, at least 75%, at least 80%, at least 85%, at least 90% or at least 95% to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 70%, at most 75%, at most 80%, at most 85%, at most 90% or at most 95% to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270. In yet other aspects of this embodiment, an IFN-α targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 1, 2, 3, 4, 5, 6, 7, 8, 9, 10, 15 or 20 non-contiguous amino acid deletions, additions, and/or substitutions relative to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270. In still other aspects of this embodiment, an IFN-α targeting domain comprises a polypeptide having, e.g., at least 1, 2, 3, 4, 5, 6, 7, 8, 9, 10, 15 or 20 contiguous amino acid deletions, additions, and/or substitutions relative to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270; or at most 1, 2, 3, 4, 5, 6, 7, 8, 9, 10, 15 or 20 contiguous amino acid deletions, additions, and/or substitutions relative to amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270.

An example of a targeting domain disclosed herein is a pigment epithelium-derived factor (PEDF) targeting domain. PEDF targeting domains bind to transmembrane receptors present on the surface of endothelial cells associated with aberrant new blood vessel formation. For example, PEDF receptors are expressed in endothelial cells undergoing angiogenesis in response to tumor progression as well as endothelial cells participating in angiogenesis and neovasculogenesis associated with several disorders including ocular disorders. As such, a TVEMP comprising a PEDF targeting domain would be effective in treating a disease or disorder associated with aberrant new blood vessel formation.

Thus, in an embodiment, a targeting domain comprises a PEDF targeting domain. In aspects of this embodiment, a PEDF targeting domain comprises SEQ ID NO: 271, SEQ ID NO: 272, or SEQ ID NO: 273. In yet other aspects of this embodiment, a PEDF targeting domain comprises amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273.

In other aspects of this embodiment, a PEDF targeting domain comprises a polypeptide having an amino acid identity of, e.g., at least 70%, at least 75%, at least 80%, at least 85%, at least 90% or at least 95% to SEQ ID NO: 271, SEQ ID NO: 272, or SEQ ID NO: 273; or at most 70%, at most 75%, at most 80%, at most 85%, at most 90% or at most 95% to SEQ ID NO: 271, SEQ ID NO: 272, or SEQ ID NO: 273. In yet other aspects of this embodiment, a PEDF targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 271, SEQ ID NO: 272, or SEQ ID NO: 273; or at most 1, 2, 3, 4, 5, 6, 7, 8, 9, 10, 15 or 20 non-contiguous amino acid deletions, additions, and/or substitutions relative to SEQ ID NO: 271, SEQ ID NO: 272, or SEQ ID NO: 273. In still other aspects of this embodiment, a PEDF targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 271, SEQ ID NO: 272, or SEQ ID NO: 273; or at most 1, 2, 3, 4, 5, 6, 7, 8, 9, 10, 15 or 20 contiguous amino acid deletions, additions, and/or substitutions relative to SEQ ID NO: 271, SEQ ID NO: 272, or SEQ ID NO: 273.

In other aspects of this embodiment, a PEDF targeting domain comprises a polypeptide having an amino acid identity of, e.g., at least 70%, at least 75%, at least 80%, at least 85%, at least 90% or at least 95% to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273; or at most 70%, at most 75%, at most 80%, at most 85%, at most 90% or at most 95% to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273. In yet other aspects of this embodiment, a PEDF targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273; or at most 1, 2, 3, 4, 5, 6, 7, 8, 9, 10, 15 or 20 non-contiguous amino acid deletions, additions, and/or substitutions relative to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273. In still other aspects of this embodiment, a PEDF targeting domain comprises a polypeptide having, e.g., at least 1, 2, 3, 4, 5, 6, 7, 8, 9, 10, 15 or 20 contiguous amino acid deletions, additions, and/or substitutions relative to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273; or at most 1, 2, 3, 4, 5, 6, 7, 8, 9, 10, 15 or 20 contiguous amino acid deletions, additions, and/or substitutions relative to amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273.

An example of a targeting domain disclosed herein is a troponin targeting domain. Troponin targeting domains bind to transmembrane receptors present on the surface of endothelial cells associated with aberrant new blood vessel formation. For example, troponin receptors like FGFR2 are expressed in endothelial cells undergoing angiogenesis in response to tumor progression. As such, a TVEMP comprising a troponin targeting domain would be effective in treating a disease or disorder associated with aberrant new blood vessel formation.

Thus, in an embodiment, a targeting domain comprises a troponin targeting domain. In aspects of this embodiment, a troponin targeting domain comprises SEQ ID NO: 274, SEQ ID NO: 275, or SEQ ID NO: 276. In yet other aspects of this embodiment, a troponin targeting domain comprises amino acids 15-132 or amino acids 28-131 of SEQ ID NO: 274, SEQ ID NO: 275, or SEQ ID NO: 276.

In other aspects of this embodiment, a troponin targeting domain comprises a polypeptide having an amino acid identity of, e.g., at least 70%, at least 75%, at least 80%, at least 85%, at least 90% or at least 95% to SEQ ID NO: 274, SEQ ID NO: 275, or SEQ ID NO: 276; or at most 70%, at most 75%, at most 80%, at most 85%, at most 90% or at most 95% to SEQ ID NO: 274, SEQ ID NO: 275, or SEQ ID NO: 276. In yet other aspects of this embodiment, a troponin targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 274, SEQ ID NO: 275, or SEQ ID NO: 276; or at most 1, 2, 3, 4, 5, 6, 7, 8, 9, 10, 15 or 20 non-contiguous amino acid deletions, additions, and/or substitutions relative to SEQ ID NO: 274, SEQ ID NO: 275, or SEQ ID NO: 276. In still other aspects of this embodiment, a troponin targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 274, SEQ ID NO: 275, or SEQ ID NO: 276; or at most 1, 2, 3, 4, 5, 6, 7, 8, 9, 10, 15 or 20 contiguous amino acid deletions, additions, and/or substitutions relative to SEQ ID NO: 274, SEQ ID NO: 275, or SEQ ID NO: 276.

In other aspects of this embodiment, a troponin targeting domain comprises a polypeptide having an amino acid identity of, e.g., at least 70%, at least 75%, at least 80%, at least 85%, at least 90% or at least 95% to amino acids 15-132 or amino acids 28-131 of SEQ ID NO: 274, SEQ ID NO: 275, or SEQ ID NO: 276; or at most 70%, at most 75%, at most 80%, at most 85%, at most 90% or at most 95% to amino acids 15-132 or amino acids 28-131 of SEQ ID NO: 274, SEQ ID NO: 275, or SEQ ID NO: 276. In yet other aspects of this embodiment, a troponin targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 15-132 or amino acids 28-131 of SEQ ID NO: 274, SEQ ID NO: 275, or SEQ ID NO: 276; or at most 1, 2, 3, 4, 5, 6, 7, 8, 9, 10, 15 or 20 non-contiguous amino acid deletions, additions, and/or substitutions relative to amino acids 15-132 or amino acids 28-131 of SEQ ID NO: 274, SEQ ID NO: 275, or SEQ ID NO: 276. In still other aspects of this embodiment, a troponin targeting domain comprises a polypeptide having, e.g., at least 1, 2, 3, 4, 5, 6, 7, 8, 9, 10, 15 or 20 contiguous amino acid deletions, additions, and/or substitutions relative to amino acids 15-132 or amino acids 28-131 of SEQ ID NO: 274, SEQ ID NO: 275, or SEQ ID NO: 276; or at most 1, 2, 3, 4, 5, 6, 7, 8, 9, 10, 15 or 20 contiguous amino acid deletions, additions, and/or substitutions relative to amino acids 15-132 or amino acids 28-131 of SEQ ID NO: 274, SEQ ID NO: 275, or SEQ ID NO: 276.

An example of a targeting domain disclosed herein is a vasohibin (VASH) targeting domain or an Urocortin (Ucn) targeting domain. Non-limiting examples of a VASH targeting domain include a vasohibin-1 targeting domain and a vasohibin-2 targeting domain. Non-limiting examples of an urocortin targeting domain include a Urocortin-2 targeting domain. VASH and Ucn targeting domains bind to transmembrane receptors present on the surface of endothelial cells associated with aberrant new blood vessel formation. For example, VASH receptors are expressed in endothelial cells undergoing angiogenesis in response to tumor progression as well as endothelial cells participating in angiogenesis and neovasculogenesis associated with several disorders including ocular disorders. As another example, Ucn receptors are expressed in endothelial cells undergoing angiogenesis in response to tumor progression. As such, a TVEMP comprising a VASH targeting domain or an Ucn targeting domain would be effective in treating a disease or disorder associated with aberrant new blood vessel formation.

Thus, in an embodiment, a targeting domain comprises a VASH targeting domain or an Ucn targeting domain. In aspects of this embodiment, a VASH targeting domain is a VASH1 targeting domain or a VASH2 targeting domain. In aspects of this embodiment, a targeting domain comprises a VASH targeting domain including SEQ ID NO: 277, SEQ ID NO: 278, SEQ ID NO: 279, SEQ ID NO: 280, SEQ ID NO: 281, SEQ ID NO: 282, or SEQ ID NO: 283; or a Ucn targeting domain including SEQ ID NO: 284, SEQ ID NO: 285, or SEQ ID NO: 286. In yet other aspects of this embodiment, a targeting domain comprises a VASH targeting domain including amino acids 319-365 of SEQ ID NO: 277 or SEQ ID NO: 278, or amino acids 329-375 of SEQ ID NO: 279; or a Ucn targeting domain including amino acids 20-112 or amino acids 72-109 of SEQ ID NO: 284 or SEQ ID NO: 285, or amino acids 21-123 or amino acids 83-119 of SEQ ID NO: 286.

In other aspects of this embodiment, a VASH targeting domain or an Ucn targeting domain comprises a polypeptide having an amino acid identity of, e.g., at least 70%, at least 75%, at least 80%, at least 85%, at least 90% or at least 95% to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286; or at most 70%, at most 75%, at most 80%, at most 85%, at most 90% or at most 95% to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286. In yet other aspects of this embodiment, a VASH targeting domain or an Ucn targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286; or at most 1, 2, 3, 4, 5, 6, 7, 8, 9, 10, 15 or 20 non-contiguous amino acid deletions, additions, and/or substitutions relative to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286. In still other aspects of this embodiment, a VASH targeting domain or an Ucn targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286; or at most 1, 2, 3, 4, 5, 6, 7, 8, 9, 10, 15 or 20 contiguous amino acid deletions, additions, and/or substitutions relative to SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, or SEQ ID NO: 286.

In other aspects of this embodiment, a VASH targeting domain or an Ucn targeting domain comprises a polypeptide having an amino acid identity of, e.g., at least 70%, at least 75%, at least 80%, at least 85%, at least 90% or at least 95% to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286; or at most 70%, at most 75%, at most 80%, at most 85%, at most 90% or at most 95% to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286. In yet other aspects of this embodiment, a VASH targeting domain or an Ucn targeting domain targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286; or at most 1, 2, 3, 4, 5, 6, 7, 8, 9, 10, 15 or 20 non-contiguous amino acid deletions, additions, and/or substitutions relative to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286. In still other aspects of this embodiment, a VASH targeting domain or an Ucn targeting domain comprises a polypeptide having, e.g., at least 1, 2, 3, 4, 5, 6, 7, 8, 9, 10, 15 or 20 contiguous amino acid deletions, additions, and/or substitutions relative to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286; or at most 1, 2, 3, 4, 5, 6, 7, 8, 9, 10, 15 or 20 contiguous amino acid deletions, additions, and/or substitutions relative to amino acids 319-365 of SEQ ID NO: 277 or SEQ ID NO: 278, amino acids 329-375 of SEQ ID NO: 279, amino acids 20-112 or amino acids 72-109 of SEQ ID NO: 284 or SEQ ID NO: 285, or amino acids 21-123 or amino acids 83-119 of SEQ ID NO: 286.

An example of a targeting domain disclosed herein is a C-motif cytokine ligand (CL) targeting domain. Non-limiting examples of a C-motif cytokine ligand targeting domain is a lymphotactin targeting domain. C-motif cytokine ligand targeting domains bind to transmembrane receptors present on the surface of endothelial cells associated with aberrant new blood vessel formation. For example, lymphotactin receptors like XCR1 are expressed in endothelial cells undergoing angiogenesis in response to tumor progression as well as endothelial cells participating in angiogenesis and neovasculogenesis associated with several disorders including ocular disorders. As such, a TVEMP comprising a C-motif cytokine ligand targeting domain would be effective in treating a disease or disorder associated with aberrant new blood vessel formation.

Thus, in an embodiment, a targeting domain comprises a CL targeting domain. In aspects of this embodiment, a CL targeting domain is a lymphotactin targeting domain. In aspects of this embodiment, a CL targeting domain comprises SEQ ID NO: 287, SEQ ID NO: 288, SEQ ID NO: 289, SEQ ID NO: 290, SEQ ID NO: 291, SEQ ID NO: 292, or SEQ ID NO: 293. In yet other aspects of this embodiment, a CL targeting domain comprises amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293.

In other aspects of this embodiment, a CL targeting domain comprises a polypeptide having an amino acid identity of, e.g., at least 70%, at least 75%, at least 80%, at least 85%, at least 90% or at least 95% to SEQ ID NO: 287, SEQ ID NO: 288, SEQ ID NO: 289, SEQ ID NO: 290, SEQ ID NO: 291, SEQ ID NO: 292, or SEQ ID NO: 293; or at most 70%, at most 75%, at most 80%, at most 85%, at most 90% or at most 95% to SEQ ID NO: 287, SEQ ID NO: 288, SEQ ID NO: 289, SEQ ID NO: 290, SEQ ID NO: 291, SEQ ID NO: 292, or SEQ ID NO: 293. In yet other aspects of this embodiment, a CL targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 287, SEQ ID NO: 288, SEQ ID NO: 289, SEQ ID NO: 290, SEQ ID NO: 291, SEQ ID NO: 292, or SEQ ID NO: 293; or at most 1, 2, 3, 4, 5, 6, 7, 8, 9, 10, 15 or 20 non-contiguous amino acid deletions, additions, and/or substitutions relative to SEQ ID NO: 287, SEQ ID NO: 288, SEQ ID NO: 289, SEQ ID NO: 290, SEQ ID NO: 291, SEQ ID NO: 292, or SEQ ID NO: 293. In still other aspects of this embodiment, a CL targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 287, SEQ ID NO: 288, SEQ ID NO: 289, SEQ ID NO: 290, SEQ ID NO: 291, SEQ ID NO: 292, or SEQ ID NO: 293; or at most 1, 2, 3, 4, 5, 6, 7, 8, 9, 10, 15 or 20 contiguous amino acid deletions, additions, and/or substitutions relative to SEQ ID NO: 287, SEQ ID NO: 288, SEQ ID NO: 289, SEQ ID NO: 290, SEQ ID NO: 291, SEQ ID NO: 292, or SEQ ID NO: 293.

In other aspects of this embodiment, a CL targeting domain comprises a polypeptide having an amino acid identity of, e.g., at least 70%, at least 75%, at least 80%, at least 85%, at least 90% or at least 95% to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293; or at most 70%, at most 75%, at most 80%, at most 85%, at most 90% or at most 95% to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293. In yet other aspects of this embodiment, a CL targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293; or at most 1, 2, 3, 4, 5, 6, 7, 8, 9, 10, 15 or 20 non-contiguous amino acid deletions, additions, and/or substitutions relative to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293. In still other aspects of this embodiment, a CL targeting domain comprises a polypeptide having, e.g., at least 1, 2, 3, 4, 5, 6, 7, 8, 9, 10, 15 or 20 contiguous amino acid deletions, additions, and/or substitutions relative to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293; or at most 1, 2, 3, 4, 5, 6, 7, 8, 9, 10, 15 or 20 contiguous amino acid deletions, additions, and/or substitutions relative to amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293.

An example of a targeting domain disclosed herein is a C—C-motif cytokine ligand (CCL) targeting domain. Non-limiting examples of a C—C-motif cytokine ligand targeting domain is a CCL1 targeting domain and a CCL2 targeting domain. C—C-motif cytokine ligand targeting domains bind to transmembrane receptors present on the surface of endothelial cells associated with aberrant new blood vessel formation. For example, CCL1 receptors like CCR8 are expressed in endothelial cells participating in angiogenesis and neovasculogenesis associated with several disorders including ocular disorders. As another example, CCL2 receptors like CCR2 are expressed in endothelial cells undergoing angiogenesis in response to tumor progression. As such, a TVEMP comprising a C—C-motif cytokine ligand targeting domain would be effective in treating a disease or disorder associated with aberrant new blood vessel formation.

Thus, in an embodiment, a targeting domain comprises a CCL targeting domain. In aspects of this embodiment, a CCL targeting domain is a CCL1 targeting domain or a CCL2 targeting domain. In aspects of this embodiment, a CCL targeting domain comprises SEQ ID NO: 294, SEQ ID NO: 295, SEQ ID NO: 296, or SEQ ID NO: 297. In yet other aspects of this embodiment, a CCL targeting domain comprises amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297.

In other aspects of this embodiment, a CCL targeting domain comprises a polypeptide having an amino acid identity of, e.g., at least 70%, at least 75%, at least 80%, at least 85%, at least 90% or at least 95% to SEQ ID NO: 294, SEQ ID NO: 295, SEQ ID NO: 296, or SEQ ID NO: 297; or at most 70%, at most 75%, at most 80%, at most 85%, at most 90% or at most 95% to SEQ ID NO: 294, SEQ ID NO: 295, SEQ ID NO: 296, or SEQ ID NO: 297. In yet other aspects of this embodiment, a CCL targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 294, SEQ ID NO: 295, SEQ ID NO: 296, or SEQ ID NO: 297; or at most 1, 2, 3, 4, 5, 6, 7, 8, 9, 10, 15 or 20 non-contiguous amino acid deletions, additions, and/or substitutions relative to SEQ ID NO: 294, SEQ ID NO: 295, SEQ ID NO: 296, or SEQ ID NO: 297. In still other aspects of this embodiment, a CCL targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 294, SEQ ID NO: 295, SEQ ID NO: 296, or SEQ ID NO: 297; or at most 1, 2, 3, 4, 5, 6, 7, 8, 9, 10, 15 or 20 contiguous amino acid deletions, additions, and/or substitutions relative to SEQ ID NO: 294, SEQ ID NO: 295, SEQ ID NO: 296, or SEQ ID NO: 297.

In other aspects of this embodiment, a CCL targeting domain comprises a polypeptide having an amino acid identity of, e.g., at least 70%, at least 75%, at least 80%, at least 85%, at least 90% or at least 95% to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297; or at most 70%, at most 75%, at most 80%, at most 85%, at most 90% or at most 95% to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297. In yet other aspects of this embodiment, a CCL targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297; or at most 1, 2, 3, 4, 5, 6, 7, 8, 9, 10, 15 or 20 non-contiguous amino acid deletions, additions, and/or substitutions relative to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297. In still other aspects of this embodiment, a CCL targeting domain comprises a polypeptide having, e.g., at least 1, 2, 3, 4, 5, 6, 7, 8, 9, 10, 15 or 20 contiguous amino acid deletions, additions, and/or substitutions relative to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297; or at most 1, 2, 3, 4, 5, 6, 7, 8, 9, 10, 15 or 20 contiguous amino acid deletions, additions, and/or substitutions relative to amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297.

An example of a targeting domain disclosed herein is a C—X—C-motif cytokine ligand (CXCL) targeting domain or an endothelial-monocyte activating polypeptide (EMAP) targeting domain. Non-limiting examples of a C—X—C-motif cytokine ligand targeting domain is a CXCL1 targeting domain, a CXCL2 targeting domain, a CXCL3 targeting domain, a CXCL4 targeting domain, a CXCL5 targeting domain, a CXCL6 targeting domain, a CXCL7 targeting domain, a CXCL8 targeting domain, a CXCL9 targeting domain, a CXCL10 targeting domain, a CXCL11 targeting domain, a CXCL12 targeting domain, a CXCL13 targeting domain, a CXCL14 targeting domain, a CXCL16 targeting domain, and an endothelial-monocyte activating polypeptide II (EMAP II) targeting domain. C—X—C-motif cytokine ligand targeting domains bind to transmembrane receptors present on the surface of endothelial cells associated with aberrant new blood vessel formation. For example, CXCL receptors like CXCR1, CXCR2, CXCR3A, CXCR3B, CXCR4, CXCR5, CXCR6 are expressed in endothelial cells undergoing angiogenesis in response to tumor progression as well as participating in angiogenesis and neovasculogenesis associated with several disorders including ocular disorders. As such, a TVEMP comprising a C—X—C-motif cytokine ligand targeting domain would be effective in treating a disease or disorder associated with aberrant new blood vessel formation.

Thus, in an embodiment, a targeting domain comprises a CXCL targeting domain. In aspects of this embodiment, a CXCL targeting domain is a CXCL1 targeting domain, a CXCL2 targeting domain, a CXCL3 targeting domain, a CXCL4 targeting domain, a CXCL5 targeting domain, a CXCL6 targeting domain, a CXCL7 targeting domain, a CXCL8 targeting domain, a CXCL9 targeting domain, a CXCL10 targeting domain, a CXCL11 targeting domain, a CXCL12 targeting domain, a CXCL13 targeting domain, a CXCL14 targeting domain, a CXCL16 targeting domain, or a EMAP targeting domain. In aspects of this embodiment, a CXCL targeting domain comprises SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379.

In yet other aspects of this embodiment, a CXCL targeting domain comprises amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379.

In other aspects of this embodiment, a CXCL targeting domain comprises a polypeptide having an amino acid identity of, e.g., at least 70%, at least 75%, at least 80%, at least 85%, at least 90% or at least 95% to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379; or at most 70%, at most 75%, at most 80%, at most 85%, at most 90% or at most 95% to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379. In yet other aspects of this embodiment, a CXCL targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379; or at most 1, 2, 3, 4, 5, 6, 7, 8, 9, 10, 15 or 20 non-contiguous amino acid deletions, additions, and/or substitutions relative to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379. In still other aspects of this embodiment, a CXCL targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379; or at most 1, 2, 3, 4, 5, 6, 7, 8, 9, 10, 15 or 20 contiguous amino acid deletions, additions, and/or substitutions relative to SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, or SEQ ID NO: 379.

In other aspects of this embodiment, a CXCL targeting domain comprises a polypeptide having an amino acid identity of, e.g., at least 70%, at least 75%, at least 80%, at least 85%, at least 90% or at least 95% to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379; or at most 70%, at most 75%, at most 80%, at most 85%, at most 90% or at most 95% to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379.

In yet other aspects of this embodiment, a CXCL targeting domain comprises a polypeptide having, e.g., at least 1, 2, 3, 4, 5, 6, 7, 8, 9, 10, 15 or 20 non-contiguous amino acid deletions, additions, and/or substitutions relative to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379; or at most 1, 2, 3, 4, 5, 6, 7, 8, 9, 10, 15 or 20 non-contiguous amino acid deletions, additions, and/or substitutions relative to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379.

In still other aspects of this embodiment, a CXCL targeting domain comprises a polypeptide having, e.g., at least 1, 2, 3, 4, 5, 6, 7, 8, 9, 10, 15 or 20 contiguous amino acid deletions, additions, and/or substitutions relative to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379; or at most 1, 2, 3, 4, 5, 6, 7, 8, 9, 10, 15 or 20 contiguous amino acid deletions, additions, and/or substitutions relative to amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379.

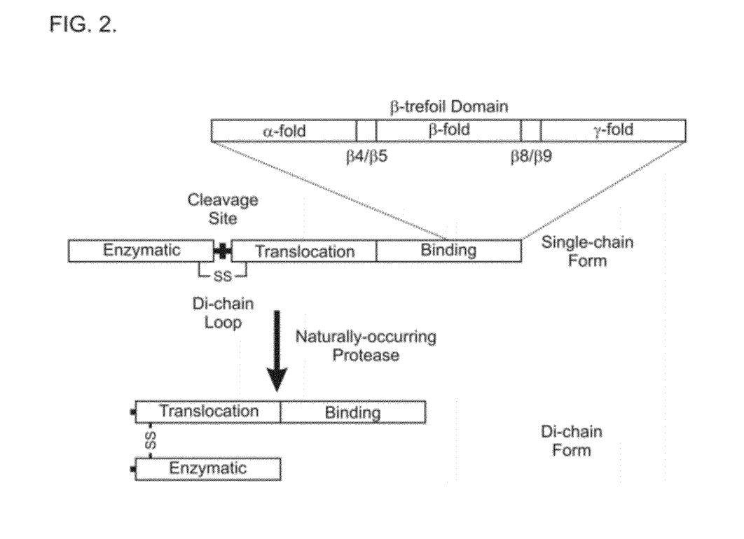

Clostridial toxins are each translated as a single-chain polypeptide of approximately 150 kDa that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains (FIG. 2). To facilitate recombinant production of a TVEMP, an exogenous protease cleavage site can be used to convert the single-chain polypeptide form of a TVEMP disclosed herein into the di-chain form. See, e.g., Steward, L. E. et al., Modified Clostridial Toxins with Enhanced Targeting Capabilities For Endogenous Clostridial Toxin Receptor Systems, U.S. Patent Publication No. US 2008/0096248 (Apr. 24, 2008); Steward, L. E. et al., Activatable Clostridial Toxins, U.S. Patent Publication No. US 2008/0032930 (Feb. 7, 2008); Steward, supra, (2007); Dolly, supra, (2007); Foster, supra, WO 2006/059093 (2006); and Foster, supra, WO 2006/059105 (2006), each of which is hereby incorporated by reference in its entirety.

It is envisioned that any and all protease cleavage sites can be used to convert the single-chain polypeptide form of a Clostridial toxin into the di-chain form, including, without limitation, endogenous di-chain loop protease cleavage sites and exogenous protease cleavage sites. Thus, in an aspect of the invention, a TVEMP comprises, in part, an endogenous protease cleavage site within a di-chain loop region. In another aspect of the invention, a TVEMP comprises, in part, an exogenous protease cleavage site within a di-chain loop region. As used herein, the term “di-chain loop region” means the amino acid sequence of a Clostridial toxin containing a protease cleavage site used to convert the single-chain form of a Clostridial toxin into the di-chain form. Non-limiting examples of a Clostridial toxin di-chain loop region, include, a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; and a di-chain loop region of TeNT comprising amino acids 439-467 of SEQ ID NO: 8 (Table 4).

TABLE 4
Di-chain Loop Region of Clostridial Toxins

| | | Di-chain Loop Region Containing |
|---|---|---|
| | SEQ | the Naturally-occurring |
| Toxin | ID NO: | Protease Cleavage Site |
| BoNT/A | 26 | CVRGIITSKTKSLDKGYNK*----ALNDLC |
| BoNT/B | 27 | CKSVK*-------------------APGIC |
| BoNT/C1 | 28 | CHKAIDGRSLYNK*------------TLDC |
| BoNT/D | 29 | CLRLTKNSR*---------------DDSTC |
| BoNT/E | 30 | CKNIVSVKGIR*--------------KSIC |
| BoNT/F | 31 | CKSVIPRKGTK*------------APPRLC |
| BoNT/G | 32 | CKPVMYKNTGK*--------------SEQC |
| TeNT | 33 | CKKIIPPTNIRENLYNRTA*SLTDLGGELC |
| BaNT | 34 | CKS-IVSKKGTK*------------NSLC |
| BuNT | 35 | CKN-IVSVKGIR*--------------KSIC |

The amino acid sequence displayed are as follows: BoNT/A, residues 430-454 of SEQ ID NO: 1;
BoNT/B, residues 437-446 of SEQ ID NO: 2; BoNT/C1, residues 437-453 of SEQ ID NO: 3; BoNT/D,
residues 437-450 of SEQ ID NO: 4; BoNT/E, residues 412-426 of SEQ ID NO: 5; BoNT/F, residues 429-
445 of SEQ ID NO: 6; BoNT/G, residues 436-450 of SEQ ID NO: 7; TeNT, residues 439-467 of SEQ ID
NO: 8; BaNT, residues 421-435 of SEQ ID NO: 9; and BuNT, residues 412-426 of SEQ ID NO: 10. An
asterisks (*) indicates the peptide bond that is cleaved by a Clostridial toxin protease.

As used herein, the term “endogenous di-chain loop protease cleavage site” is synonymous with a “naturally occurring di-chain loop protease cleavage site” and means a naturally occurring protease cleavage site found within the di-chain loop region of a naturally occurring Clostridial toxin and includes, without limitation, naturally occurring Clostridial toxin di-chain loop protease cleavage site variants, such as, e.g., Clostridial toxin di-chain loop protease cleavage site isoforms and Clostridial toxin di-chain loop protease cleavage site subtypes. Non-limiting examples of an endogenous protease cleavage site, include, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site and a TeNT di-chain loop protease cleavage site.

As mentioned above, Clostridial toxins are translated as a single-chain polypeptide of approximately 150 kDa that is subsequently cleaved by proteolytic scission within a disulfide loop by a naturally-occurring protease. This posttranslational processing yields a di-chain molecule comprising an approximately 50 kDa light chain (LC) and an approximately 100 kDa heavy chain (HC) held together by a single disulphide bond and noncovalent interactions. While the identity of the protease is currently unknown, the di-chain loop protease cleavage site for many Clostridial toxins has been determined. In BoNTs, cleavage at K448-A449 converts the single polypeptide form of BoNT/A into the di-chain form; cleavage at K441-A442 converts the single polypeptide form of BoNT/B into the di-chain form; cleavage at K449-T450 converts the single polypeptide form of BoNT/C1 into the di-chain form; cleavage at R445-D446 converts the single polypeptide form of BoNT/D into the di-chain form; cleavage at R422-K423 converts the single polypeptide form of BoNT/E into the di-chain form; cleavage at K439-A440 converts the single polypeptide form of BoNT/F into the di-chain form; and cleavage at K446-S447 converts the single polypeptide form of BoNT/G into the di-chain form. Proteolytic cleavage of the single polypeptide form of TeNT at A457-S458 results in the di-chain form. Proteolytic cleavage of the single polypeptide form of BaNT at K431-N432 results in the di-chain form. Proteolytic cleavage of the single polypeptide form of BuNT at R422-K423 results in the di-chain form. Such a di-chain loop protease cleavage site is operably-linked in-frame to a TVEMP as a fusion protein. However, it should also be noted that additional cleavage sites within the di-chain loop also appear to be cleaved resulting in the generation of a small peptide fragment being lost. As a non-limiting example, BoNT/A single-chain polypeptide cleave ultimately results in the loss of a ten amino acid fragment within the di-chain loop.

Thus, in an embodiment, a protease cleavage site comprising an endogenous Clostridial toxin di-chain loop protease cleavage site is used to convert the single-chain toxin into the di-chain form. In aspects of this embodiment, conversion into the di-chain form by proteolytic cleavage occurs from a site comprising, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site, a TeNT di-chain loop protease cleavage site, a BaNT di-chain loop protease cleavage site, or a BuNT di-chain loop protease cleavage site.

In other aspects of this embodiment, conversion into the di-chain form by proteolytic cleavage occurs from a site comprising, e.g., a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; or a di-chain loop region of TeNT comprising amino acids 439-467 of SEQ ID NO: 8. a di-chain loop region of BaNT comprising amino acids 421-435 of SEQ ID NO: 9; or a di-chain loop region of BuNT comprising amino acids 412-426 of SEQ ID NO: 10.

It is also envisioned that an exogenous protease cleavage site can be used to convert the single-chain polypeptide form of a TVEMP disclosed herein into the di-chain form. As used herein, the term “exogenous protease cleavage site” is synonymous with a “non-naturally occurring protease cleavage site” or “non-native protease cleavage site” and means a protease cleavage site that is not normally present in a di-chain loop region from a naturally occurring Clostridial toxin, with the proviso that the exogenous protease cleavage site is not a human protease cleavage site or a protease cleavage site that is susceptible to a protease being expressed in the host cell that is expressing a construct encoding an activatable polypeptide disclosed herein. It is envisioned that any and all exogenous protease cleavage sites can be used to convert the single-chain polypeptide form of a Clostridial toxin into the di-chain form are useful to practice aspects of the present invention. Non-limiting examples of exogenous protease cleavage sites include, e.g., a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus (TEV) protease cleavage site, a Tobacco Vein Mottling Virus (TVMV) cleavage site, a subtilisin cleavage site, a hydroxylamine cleavage site, or a Caspase 3 cleavage site.

It is envisioned that an exogenous protease cleavage site of any and all lengths can be useful in aspects of the present invention with the proviso that the exogenous protease cleavage site is capable of being cleaved by its respective protease. Thus, in aspects of this embodiment, an exogenous protease cleavage site can have a length of, e.g., at least 6, 7, 8, 9, 10, 15, 20, 25, 30, 40, 50, or at least 60 amino acids; or at most 6, 7, 8, 9, 10, 15, 20, 25, 30, 40, 50, or at least 60 amino acids.

In an embodiment, an exogenous protease cleavage site is located within the di-chain loop of a TVEMP. In aspects of this embodiment, a TVEMP comprises an exogenous protease cleavage site comprises, e.g., a plant papain cleavage site, an insect papain cleavage site, a crustacian papain cleavage site, a non-human enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a non-human Caspase 3 cleavage site. In other aspects of this embodiment, an exogenous protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human enterokinase cleavage site is located within the di-chain loop of a TVEMP. In other aspects of the embodiment, an exogenous protease cleavage site can comprise, e.g., a bovine enterokinase protease cleavage site located within the di-chain loop of a TVEMP. In other aspects of the embodiment, an exogenous protease cleavage site can comprise, e.g., a bovine enterokinase protease cleavage site located within the di-chain loop of a TVEMP comprises SEQ ID NO: 36. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Etch Virus protease cleavage site is located within the di-chain loop of a TVEMP. In other aspects of the embodiment, an exogenous protease cleavage site can comprise, e.g., a Tobacco Etch Virus protease cleavage site located within the di-chain loop of a TVEMP comprises the consensus sequence E-P5-P4-Y-P2-Q*-G (SEQ ID NO: 377) or E-P5-P4-Y-P2-Q*-S (SEQ ID NO: 38), where P2, P4 and P5 can be any amino acid. In other aspects of the embodiment, an exogenous protease cleavage site can comprise, e.g., a Tobacco Etch Virus protease cleavage site located within the di-chain loop of a TVEMP comprises SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48. In still other aspects of this embodiment, a Tobacco Etch Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Vein Mottling Virus protease cleavage site is located within the di-chain loop of a TVEMP. In other aspects of the embodiment, an exogenous protease cleavage site can comprise, e.g., a Tobacco Vein Mottling Virus protease cleavage site located within the di-chain loop of a TVEMP comprises the consensus sequence P6-P5-V-R-F-Q*-G (SEQ ID NO: 49) or P6-P5-V-R-F-Q*-S (SEQ ID NO: 50), where P5 and P6 can be any amino acid. In other aspects of the embodiment, an exogenous protease cleavage site can comprise, e.g., a Tobacco Vein Mottling Virus protease cleavage site located within the di-chain loop of a TVEMP comprises SEQ ID NO: 51, SEQ ID NO: 52, SEQ ID NO: 53, or SEQ ID NO: 54. In still other aspects of this embodiment, a Tobacco Vein Mottling Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In still another aspect of this embodiment, an exogenous protease cleavage site can comprise, e.g., a human rhinovirus 3C protease cleavage site is located within the di-chain loop of a TVEMP. In other aspects of the embodiment, an exogenous protease cleavage site can comprise, e.g., a human rhinovirus 3C protease cleavage site located within the di-chain loop of a TVEMP comprises the consensus sequence P5-P4-L-F-Q*-G-P (SEQ ID NO: 55), where P4 is G, A, V, L, I, M, S or T and P5 can any amino acid, with D or E preferred. In other aspects of the embodiment, an exogenous protease cleavage site can comprise, e.g., a human rhinovirus 3C protease cleavage site located within the di-chain loop of a TVEMP comprises SEQ ID NO: 56, SEQ ID NO: 57, SEQ ID NO: 58, SEQ ID NO: 59, SEQ ID NO: 60 or SEQ ID NO: 61. In other aspects of the embodiment, an exogenous protease cleavage site can comprise, e.g., a human rhinovirus 3C protease located within the di-chain loop of a TVEMP that can be cleaved by PRESCISSION®. In still other aspects of this embodiment, a human rhinovirus 3C protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a subtilisin cleavage site is located within the di-chain loop of a TVEMP. In other aspects of the embodiment, an exogenous protease cleavage site can comprise, e.g., a subtilisin cleavage site located within the di-chain loop of a TVEMP comprises the consensus sequence P6-P5-P4-P3-H*-Y (SEQ ID NO: 62) or P6-P5-P4-P3-Y-H* (SEQ ID NO: 63), where P3, P4 and P5 and P6 can be any amino acid. In other aspects of the embodiment, an exogenous protease cleavage site can comprise, e.g., a subtilisin cleavage site located within the di-chain loop of a TVEMP comprises SEQ ID NO: 64, SEQ ID NO: 65, or SEQ ID NO: 66. In other aspects of the embodiment, an exogenous protease cleavage site can comprise, e.g., a subtilisin cleavage site located within the di-chain loop of a TVEMP that can be cleaved by GENENASE®. In still other aspects of this embodiment, a subtilisin cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a hydroxylamine cleavage site is located within the di-chain loop of a TVEMP. In other aspects of the embodiment, an exogenous protease cleavage site can comprise, e.g., a hydroxylamine cleavage site comprising multiples of the dipeptide N*G. In other aspects of the embodiment, an exogenous protease cleavage site can comprise, e.g., a hydroxylamine cleavage site located within the di-chain loop of a TVEMP comprises SEQ ID NO: 67, or SEQ ID NO: 68. In still other aspects of this embodiment, a hydroxylamine cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a SUMO/ULP-1 protease cleavage site is located within the di-chain loop of a TVEMP. In other aspects of the embodiment, an exogenous protease cleavage site can comprise, e.g., a SUMO/ULP-1 protease cleavage site located within the di-chain loop of a TVEMP comprising the consensus sequence G-G*-P1′-P2′-P3′ (SEQ ID NO: 69), where P1′, P2′, and P3′ can be any amino acid. In other aspects of the embodiment, an exogenous protease cleavage site can comprise, e.g., a SUMO/ULP-1 protease cleavage site located within the di-chain loop of a TVEMP comprises SEQ ID NO: 70. In still other aspects of this embodiment, a SUMO/ULP-1 protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human Caspase 3 cleavage site is located within the di-chain loop of a TVEMP. In other aspects of the embodiment, an exogenous protease cleavage site can comprise, e.g., a mouse Caspase 3 protease cleavage site located within the di-chain loop of a TVEMP. In other aspects of the embodiment, an exogenous protease cleavage site can comprise, e.g., a non-human Caspase 3 protease cleavage site located within the di-chain loop of a TVEMP comprises the consensus sequence D-P3-P2-D*P1′ (SEQ ID NO: 71), where P3 can be any amino acid, with E preferred, P2 can be any amino acid and P1′ can any amino acid, with G or S preferred. In other aspects of the embodiment, an exogenous protease cleavage site can comprise, e.g., a non-human Caspase 3 protease cleavage site located within the di-chain loop of a TVEMP comprising SEQ ID NO: 72, SEQ ID NO: 73, SEQ ID NO: 74, SEQ ID NO: 75, SEQ ID NO: 76, or SEQ ID NO: 77. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

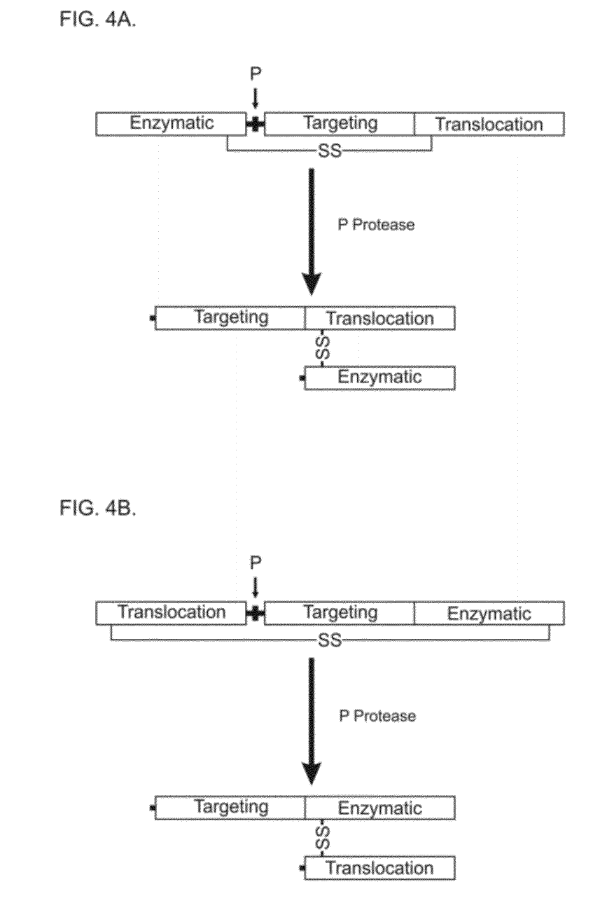

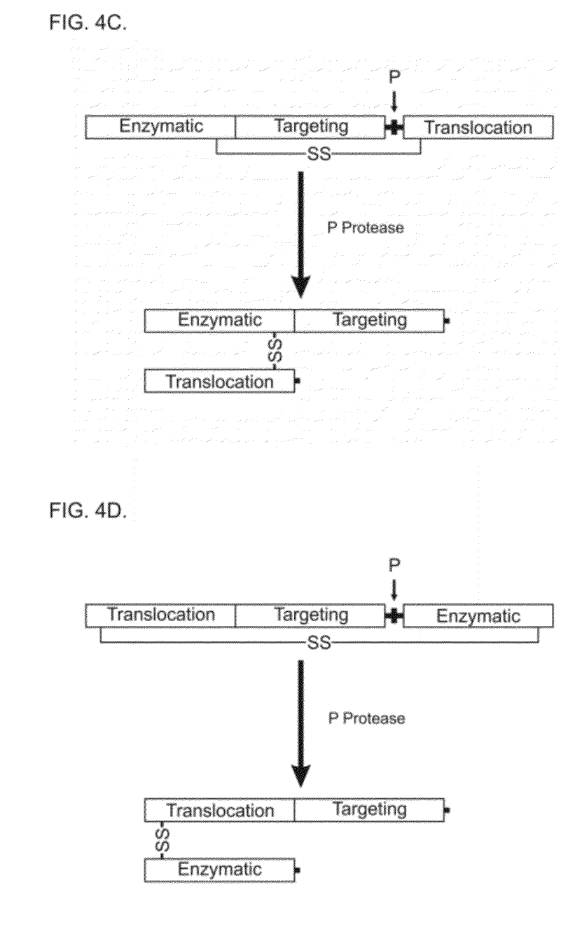

A di-chain loop region is modified to replace a naturally-occurring di-chain loop protease cleavage site for an exogenous protease cleavage site. In this modification, the naturally-occurring di-chain loop protease cleavage site is made inoperable and thus can not be cleaved by its protease. Only the exogenous protease cleavage site can be cleaved by its corresponding exogenous protease. In this type of modification, the exogenous protease site is operably-linked in-frame to a TVEMP as a fusion protein and the site can be cleaved by its respective exogenous protease. Replacement of an endogenous di-chain loop protease cleavage site with an exogenous protease cleavage site can be a substitution of the sites where the exogenous site is engineered at the position approximating the cleavage site location of the endogenous site. Replacement of an endogenous di-chain loop protease cleavage site with an exogenous protease cleavage site can be an addition of an exogenous site where the exogenous site is engineered at the position different from the cleavage site location of the endogenous site, the endogenous site being engineered to be inoperable. The location and kind of protease cleavage site may be critical because certain targeting domains require a free amino-terminal or carboxyl-terminal amino acid. For example, when a targeting domain is placed between two other domains, e.g., see FIG. 4, a criterion for selection of a protease cleavage site could be whether the protease that cleaves its site leaves a flush cut, exposing the free amino-terminal or carboxyl-terminal of the targeting domain necessary for selective binding of the targeting domain to its receptor.

A naturally-occurring protease cleavage site can be made inoperable by altering at least one of the two amino acids flanking the peptide bond cleaved by the naturally-occurring di-chain loop protease. More extensive alterations can be made, with the proviso that the two cysteine residues of the di-chain loop region remain intact and the region can still form the disulfide bridge. Non-limiting examples of an amino acid alteration include deletion of an amino acid or replacement of the original amino acid with a different amino acid. Thus, in one embodiment, a naturally-occurring protease cleavage site is made inoperable by altering at least 1, 2, 3, 4, 5, 6, 7, 8, 9, 10, 15, 20 amino acids including at least one of the two amino acids flanking the peptide bond cleaved by a naturally-occurring protease. In another embodiment, a naturally-occurring protease cleavage site is made inoperable by altering at most 1, 2, 3, 4, 5, 6, 7, 8, 9, 10, 15, 20 amino acids including at least one of the two amino acids flanking the peptide bond cleaved by a naturally-occurring protease.

It is understood that a TVEMP disclosed herein can optionally further comprise a flexible region comprising a flexible spacer. A flexible region comprising flexible spacers can be used to adjust the length of a polypeptide region in order to optimize a characteristic, attribute or property of a polypeptide. As a non-limiting example, a polypeptide region comprising one or more flexible spacers in tandem can be use to better expose a protease cleavage site thereby facilitating cleavage of that site by a protease. As another non-limiting example, a polypeptide region comprising one or more flexible spacers in tandem can be use to better present a targeting domain, thereby facilitating the binding of that targeting domain to its receptor.

A flexible space comprising a peptide is at least one amino acid in length and comprises non-charged amino acids with small side-chain R groups, such as, e.g., glycine, alanine, valine, leucine or serine. Thus, in an embodiment a flexible spacer can have a length of, e.g., at least 1, 2, 3, 4, 5, 6, 7, 8, 9, or 10 amino acids; or at most 1, 2, 3, 4, 5, 6, 7, 8, 9, or 10 amino acids. In still another embodiment, a flexible spacer can be, e.g., between 1-3 amino acids, between 2-4 amino acids, between 3-5 amino acids, between 4-6 amino acids, or between 5-7 amino acids. Non-limiting examples of a flexible spacer include, e.g., a G-spacers such as GGG, GGGG (SEQ ID NO: 78), and GGGGS (SEQ ID NO: 79) or an A-spacers such as AAA, AAAA (SEQ ID NO: 80) and AAAAV (SEQ ID NO: 81). Such a flexible region is operably-linked in-frame to the TVEMP as a fusion protein.

Thus, in an embodiment, a TVEMP disclosed herein can further comprise a flexible region comprising a flexible spacer. In another embodiment, a TVEMP disclosed herein can further comprise flexible region comprising a plurality of flexible spacers in tandem. In aspects of this embodiment, a flexible region can comprise in tandem, e.g., at least 1, 2, 3, 4, or 5 G-spacers; or at most 1, 2, 3, 4, or 5 G-spacers. In still other aspects of this embodiment, a flexible region can comprise in tandem, e.g., at least 1, 2, 3, 4, or 5 A-spacers; or at most 1, 2, 3, 4, or 5 A-spacers. In another aspect of this embodiment, a TVEMP can comprise a flexible region comprising one or more copies of the same flexible spacers, one or more copies of different flexible-spacer regions, or any combination thereof.

In other aspects of this embodiment, a TVEMP comprising a flexible spacer can be, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

It is envisioned that a TVEMP disclosed herein can comprise a flexible spacer in any and all locations with the proviso that TVEMP is capable of performing the intoxication process. In aspects of this embodiment, a flexible spacer is positioned between, e.g., an enzymatic domain and a translocation domain, an enzymatic domain and a targeting domain, an enzymatic domain and an exogenous protease cleavage site. In other aspects of this embodiment, a G-spacer is positioned between, e.g., an enzymatic domain and a translocation domain, an enzymatic domain and a targeting domain, an enzymatic domain and an exogenous protease cleavage site. In other aspects of this embodiment, an A-spacer is positioned between, e.g., an enzymatic domain and a translocation domain, an enzymatic domain and a targeting domain, an enzymatic domain and an exogenous protease cleavage site.

In other aspects of this embodiment, a flexible spacer is positioned between, e.g., a targeting domain and a translocation domain, a targeting domain and an enzymatic domain, a targeting domain and an exogenous protease cleavage site. In other aspects of this embodiment, a G-spacer is positioned between, e.g., a targeting domain and a translocation domain, a targeting domain and an enzymatic domain, a targeting domain and an exogenous protease cleavage site. In other aspects of this embodiment, an A-spacer is positioned between, e.g., a targeting domain and a translocation domain, a targeting domain and an enzymatic domain, a targeting domain and an exogenous protease cleavage site.

In yet other aspects of this embodiment, a flexible spacer is positioned between, e.g., a translocation domain and an enzymatic domain, a translocation domain and a targeting domain, a translocation domain and an exogenous protease cleavage site. In other aspects of this embodiment, a G-spacer is positioned between, e.g., a translocation domain and an enzymatic domain, a translocation domain and a targeting domain, a translocation domain and an exogenous protease cleavage site. In other aspects of this embodiment, an A-spacer is positioned between, e.g., a translocation domain and an enzymatic domain, a translocation domain and a targeting domain, a translocation domain and an exogenous protease cleavage site.

It is envisioned that a TVEMP disclosed herein can comprise a targeting domain in any and all locations with the proviso that TVEMP is capable of performing the intoxication process. Non-limiting examples include, locating a targeting domain at the amino terminus of a TVEMP; locating a targeting domain between a Clostridial toxin enzymatic domain and a translocation domain of a TVEMP; and locating a targeting domain at the carboxyl terminus of a TVEMP. Other non-limiting examples include, locating a targeting domain between a Clostridial toxin enzymatic domain and a Clostridial toxin translocation domain of a TVEMP. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a targeting domain is in the amino-terminal position, an amino acid sequence comprising a start methionine and a protease cleavage site may be operably-linked in situations in which a targeting domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

Thus, in an embodiment, a TVEMP can comprise an amino to carboxyl single polypeptide linear order comprising a targeting domain, a translocation domain, an exogenous protease cleavage site and an enzymatic domain (FIG. 3A). In an aspect of this embodiment, a TVEMP can comprise an amino to carboxyl single polypeptide linear order comprising a targeting domain, a Clostridial toxin translocation domain, an exogenous protease cleavage site and a Clostridial toxin enzymatic domain.

In another embodiment, a TVEMP can comprise an amino to carboxyl single polypeptide linear order comprising a targeting domain, an enzymatic domain, an exogenous protease cleavage site, and a translocation domain (FIG. 3B). In an aspect of this embodiment, a TVEMP can comprise an amino to carboxyl single polypeptide linear order comprising a targeting domain, a Clostridial toxin enzymatic domain, an exogenous protease cleavage site, a Clostridial toxin translocation domain.

In yet another embodiment, a TVEMP can comprise an amino to carboxyl single polypeptide linear order comprising an enzymatic domain, an exogenous protease cleavage site, a targeting domain, and a translocation domain (FIG. 4A). In an aspect of this embodiment, a TVEMP can comprise an amino to carboxyl single polypeptide linear order comprising a Clostridial toxin enzymatic domain, an exogenous protease cleavage site, a targeting domain, and a Clostridial toxin translocation domain.

In yet another embodiment, a TVEMP can comprise an amino to carboxyl single polypeptide linear order comprising a translocation domain, an exogenous protease cleavage site, a targeting domain, and an enzymatic domain (FIG. 4B). In an aspect of this embodiment, a TVEMP can comprise an amino to carboxyl single polypeptide linear order comprising a Clostridial toxin translocation domain, a targeting domain, an exogenous protease cleavage site and a Clostridial toxin enzymatic domain.

In another embodiment, a TVEMP can comprise an amino to carboxyl single polypeptide linear order comprising an enzymatic domain, a targeting domain, an exogenous protease cleavage site, and a translocation domain (FIG. 4C). In an aspect of this embodiment, a TVEMP can comprise an amino to carboxyl single polypeptide linear order comprising a Clostridial toxin enzymatic domain, a targeting domain, an exogenous protease cleavage site, a Clostridial toxin translocation domain.

In yet another embodiment, a TVEMP can comprise an amino to carboxyl single polypeptide linear order comprising a translocation domain, a targeting domain, an exogenous protease cleavage site and an enzymatic domain (FIG. 4D). In an aspect of this embodiment, a TVEMP can comprise an amino to carboxyl single polypeptide linear order comprising a Clostridial toxin translocation domain, a targeting domain, an exogenous protease cleavage site and a Clostridial toxin enzymatic domain.

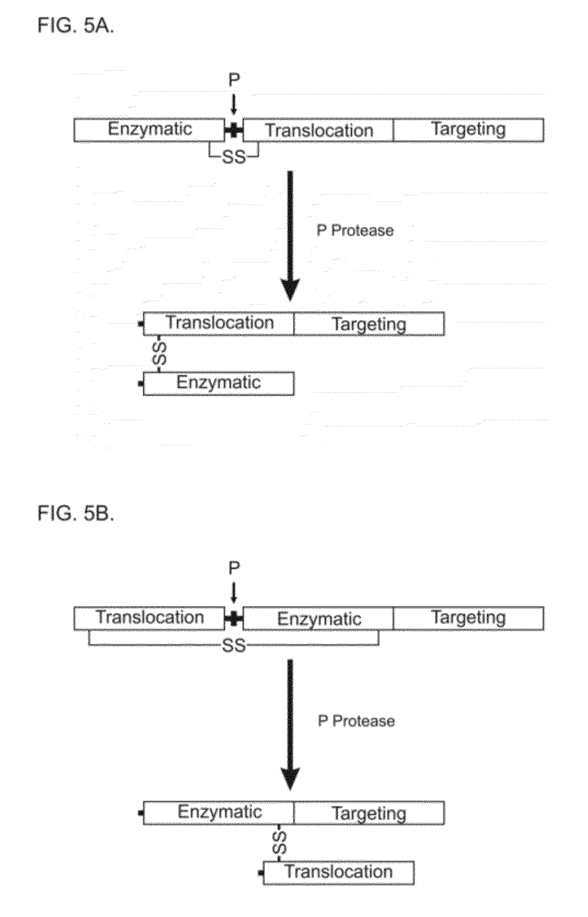

In still another embodiment, a TVEMP can comprise an amino to carboxyl single polypeptide linear order comprising an enzymatic domain, an exogenous protease cleavage site, a translocation domain, and a targeting domain (FIG. 5A). In an aspect of this embodiment, a TVEMP can comprise an amino to carboxyl single polypeptide linear order comprising a Clostridial toxin enzymatic domain, an exogenous protease cleavage site, a Clostridial toxin translocation domain, and a targeting domain.

In still another embodiment, a TVEMP can comprise an amino to carboxyl single polypeptide linear order comprising a translocation domain, an exogenous protease cleavage site, an enzymatic domain and a targeting domain, (FIG. 5B). In an aspect of this embodiment, a TVEMP can comprise an amino to carboxyl single polypeptide linear order comprising a Clostridial toxin translocation domain, a targeting domain, an exogenous protease cleavage site and a Clostridial toxin enzymatic domain.

A composition useful in the invention generally is administered as a pharmaceutical acceptable composition comprising a TVEMP. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the TVEMPs disclosed herein. A pharmaceutical composition comprising a TVEMP is useful for medical and veterinary applications. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

Aspects of the present invention provide, in part, a composition comprising a TVEMP. It is envisioned that any of the composition disclosed herein can be useful in a method of treating neurogenic inflammation in a mammal in need thereof, with the proviso that the composition prevents or reduces a symptom associated with neurogenic inflammation. Non-limiting examples of compositions comprising a TVEMP include a TVEMP comprising a targeting domain, a Clostridial toxin translocation domain and a Clostridial toxin enzymatic domain. It is envisioned that any TVEMP disclosed herein can be used, including those disclosed in, e.g., Steward, supra, (2007); Dolly, supra, (2007); Foster, supra, WO 2006/059093 (2006); Foster, supra, WO 2006/059105 (Jun. 8, 2006). It is also understood that the two or more different TVEMPs can be provided as separate compositions or as part of a single composition.

It is also envisioned that a pharmaceutical composition comprising a TVEMP can optionally include a pharmaceutically acceptable carriers that facilitate processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN′S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

It is further envisioned that a pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

In an embodiment, a composition comprising a TVEMP is a pharmaceutical composition comprising a TVEMP. In aspects of this embodiment, a pharmaceutical composition comprising a TVEMP further comprises a pharmacological carrier, a pharmaceutical component, or both a pharmacological carrier and a pharmaceutical component. In other aspects of this embodiment, a pharmaceutical composition comprising a TVEMP further comprises at least one pharmacological carrier, at least one pharmaceutical component, or at least one pharmacological carrier and at least one pharmaceutical component.

Aspects of the present invention provide, in part, a disease or disorder associated with aberrant new blood vessel formation. As used herein, the term “disease or disorder associated with aberrant new blood vessel formation” means any disease or disorder where a pathophysiology effect is aberrant new blood vessel formation. A disease or disorder associated with aberrant new blood vessel formation includes, without limitation, a retinopathy (like diabetic retinopathy), a macula degeneration (like a wet or dry macula degeneration), a choroidal neovascularization, an atherosclerosis, a coronary atherosclerotic plaque formation, an endometriosis, an idiopathic pulmonary fibrosis, chronic inflammatory/fibroproliferative disorder, a rheumatoid arthritis, a psoriasis, or a cancer. As used herein, the term “aberrant new blood vessel formation” means the formation of new blood vessels in response to a pathophysiology condition. Aberrant new blood vessel formation can be a result of angiogenesis (sprouting or splitting) or vasculogenesis. It is envisioned that the TVEMPs, compositions and methods disclosed herein can be useful to treat any disease or disorder associated with aberrant new blood vessel formation wherein the target cell is expressing the cognate receptor for the targeting domain present in the TVEMP.

Aspects of the present invention provide, in part, reducing a symptom associated with aberrant new blood vessel formation. In an aspect, the symptom reduced is an increase in the growth rate of endothelial cells, endothelial progenitor cells, tip cells, stalk cells, phalanx cells, mural cells, pericytes, and/or macrophages. In another aspect, the symptom reduced is an increase in the cell division rate of endothelial cells, endothelial progenitor cells, tip cells, stalk cells, phalanx cells, mural cells, pericytes, and/or macrophages. In yet another aspect, the symptom reduced is an increase in the extent of migration of endothelial cells, endothelial progenitor cells, tip cells, stalk cells, phalanx cells, mural cells, pericytes, and/or macrophages into the stroma. In still another aspect, the symptom reduced is an increase in angiogenesis. In a further aspect, the symptom reduced is an increase in vasculogenesis. In a yet further aspect, the symptom reduced is a decrease in apoptosis of endothelial cells, endothelial progenitor cells, tip cells, stalk cells, phalanx cells, mural cells, pericytes, and/or macrophages. In a still further aspect, the symptom reduced is a decrease in cell death or cell necrosis of endothelial cells, endothelial progenitor cells, tip cells, stalk cells, phalanx cells, mural cells, pericytes, and/or macrophages. Thus, a TVEMP treatment will decrease the growth rate of endothelial cells, endothelial progenitor cells, tip cells, stalk cells, phalanx cells, mural cells, pericytes, and/or macrophages, decrease the cell division rate of endothelial cells, endothelial progenitor cells, tip cells, stalk cells, phalanx cells, mural cells, pericytes, and/or macrophages, decrease the extent of migration of endothelial cells, endothelial progenitor cells, tip cells, stalk cells, phalanx cells, mural cells, pericytes, and/or macrophages into adjacent areas, decrease angiogenesis, decrease vasculogenesis, increase apoptosis of endothelial cells, endothelial progenitor cells, tip cells, stalk cells, phalanx cells, mural cells, pericytes, and/or macrophages, and/or increase cell death and/or cell necrosis of endothelial cells, endothelial progenitor cells, tip cells, stalk cells, phalanx cells, mural cells, pericytes, and/or macrophages.

Aspects of the present invention provide, in part, a mammal. A mammal includes a human, and a human can be a patient. Other aspects of the present invention provide, in part, an individual. An individual includes a human, and a human can be a patient.

Aspects of the present invention provide, in part, administering a composition comprising a TVEMP. As used herein, the term “administering” means any delivery mechanism that provides a composition comprising a TVEMP to a patient that potentially results in a clinically, therapeutically, or experimentally beneficial result. A TVEMP can be delivered to a patient using a cellular uptake approach where a TVEMP is delivered intracellular or a gene therapy approach where a TVEMP is express derived from precursor RNAs expressed from an expression vectors.

A composition comprising a TVEMP as disclosed herein can be administered to a mammal using a cellular uptake approach. Administration of a composition comprising a TVEMP using a cellular uptake approach comprise a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A composition comprising a TVEMP can be administered to a mammal by a variety of methods known to those of skill in the art, including, but not restricted to, encapsulation in liposomes, by ionophoresis, or by incorporation into other vehicles, such as hydrogels, cyclodextrins, biodegradable nanocapsules, and bioadhesive microspheres, or by proteinaceous vectors. Delivery mechanisms for administering a composition comprising a TVEMP to a patient are described in, e.g., Leonid Beigelman et al., Compositions for the Delivery of Negatively Charged Molecules, U.S. Pat. No. 6,395,713; and Achim Aigner, Delivery Systems for the Direct Application of siRNAs to Induce RNA Interference (RNAi) in vivo, 2006(716559) J. Biomed. Biotech. 1-15 (2006); Controlled Drug Delivery: Designing Technologies for the Future (Kinam Park & Randy J. Mrsny eds., American Chemical Association, 2000); Vernon G. Wong & Mae W. L. Hu, Methods for Treating Inflammation-mediated Conditions of the Eye, U.S. Pat. No. 6,726,918; David A. Weber et al., Methods and Apparatus for Delivery of Ocular Implants, U.S. Patent Publication No. US2004/0054374; Thierry Nivaggioli et al., Biodegradable Ocular Implant, U.S. Patent Publication No. US2004/0137059; Patrick M. Hughes et al., Anti-Angiogenic Sustained Release Intraocular Implants and Related Methods, U.S. patent application Ser. No. 11/364,687; and Patrick M. Hughes et al., Sustained Release Intraocular Drug Delivery Systems, U.S. Patent Publication 2006/0182783, each of which is hereby incorporated by reference in its entirety.

A composition comprising a TVEMP as disclosed herein can also be administered to a patient using a gene therapy approach by expressing a TVEMP within in a cell involved in the aberrant formation of new blood vessels. A TVEMP can be expressed from nucleic acid molecules operably-linked to an expression vector, see, e.g., P. D. Good et al., Expression of Small, Therapeutic RNAs in Human Cell Nuclei, 4(1) Gene Ther. 45-54 (1997); James D. Thompson, Polymerase III-based expression of therapeutic RNAs, U.S. Pat. No. 6,852,535 (Feb. 8, 2005); Maciej Wiznerowicz et al., Tuning Silence: Conditional Systems for RNA Interference, 3(9) Nat. Methods 682-688m (2006); Ola Snøve and John J. Rossi, Expressing Short Hairpin RNAi in vivo, 3(9) Nat. Methods 689-698 (2006); and Charles X. Li et al., Delivery of RNA Interference, 5(18) Cell Cycle 2103-2109 (2006). A person of ordinary skill in the art would realize that any TVEMP can be expressed in eukaryotic cells using an appropriate expression vector.

Expression vectors capable of expressing a TVEMP can provide persistent or stable expression of the TVEMP in a cell involved in the aberrant formation of new blood vessels. Alternatively, expression vectors capable of expressing a TVEMP can provide for transient expression of the TVEMP in a cell involved in the aberrant formation of new blood vessels. Such transiently expressing vectors can be repeatedly administered as necessary. A TVEMP-expressing vectors can be administered by a delivery mechanism and route of administration discussed above, by administration to target cells ex-planted from a patient followed by reintroduction into the patient, or by any other means that would allow for introduction into the desired target cell, see, e.g., Larry A. Couture and Dan T. Stinchcomb, Anti-gene Therapy: The Use of Ribozymes to Inhibit Gene Function, 12(12) Trends Genet. 510-515 (1996).

The actual delivery mechanism used to administer a composition comprising a TVEMP to a mammal can be determined by a person of ordinary skill in the art by taking into account factors, including, without limitation, the type of aberrant new blood vessel formation, the location of the aberrant new blood vessel formation, the cause of the aberrant new blood vessel formation, the severity of the aberrant new blood vessel formation, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof.

In an embodiment, a composition comprising a TVEMP is administered to the site to be treated by injection. In aspects of this embodiment, injection of a composition comprising a TVEMP is by, e.g., intramuscular injection, intraorgan injection, subdermal injection, dermal injection, or injection into any other body area for the effective administration of a composition comprising a TVEMP. In aspects of this embodiment, injection of a composition comprising a TVEMP is a region of a disease or disorder associated with aberrant new blood vessel formation or into the area surrounding such a region.

A composition comprising a TVEMP can be administered to a mammal using a variety of routes. Routes of administration suitable for a method of treating a disease or disorder associated with aberrant new blood vessel formation as disclosed herein include both local and systemic administration. Local administration results in significantly more delivery of a composition to a specific location as compared to the entire body of the mammal, whereas, systemic administration results in delivery of a composition to essentially the entire body of the patient. Routes of administration suitable for a method of treating a disease or disorder associated with aberrant new blood vessel as disclosed herein also include both central and peripheral administration. Central administration results in delivery of a composition to essentially the central nervous system of the patient and includes, e.g., intrathecal administration, epidural administration as well as a cranial injection or implant. Peripheral administration results in delivery of a composition to essentially any area of a patient outside of the central nervous system and encompasses any route of administration other than direct administration to the spine or brain. The actual route of administration of a composition comprising a TVEMP used in a mammal can be determined by a person of ordinary skill in the art by taking into account factors, including, without limitation, the type of aberrant new blood vessel, the location of the aberrant new blood vessel, the cause of the associated with aberrant new blood vessel, the severity of the aberrant new blood vessel, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the mammal, such as, e.g., age, weight, general health and the like, or any combination thereof.

In an embodiment, a composition comprising a TVEMP is administered systemically to a mammal. In another embodiment, a composition comprising a TVEMP is administered locally to a mammal. In an aspect of this embodiment, a composition comprising a TVEMP is administered to a disease or disorder associated with aberrant new blood vessel formation of a mammal. In another aspect of this embodiment, a composition comprising a TVEMP is administered to the area surrounding a disease or disorder associated with aberrant new blood vessel formation of a mammal.

Aspects of the present invention provide, in part, administering a therapeutically effective amount of a composition comprising a TVEMP. As used herein, the term “therapeutically effective amount” is synonymous with “therapeutically effective dose” and when used in reference to treating a disease or disorder associated with aberrant new blood vessel formation means the minimum dose of a TVEMP necessary to achieve the desired therapeutic effect and includes a dose sufficient to reduce or inhibit aberrant new blood vessel formation. In aspects of this embodiment, a therapeutically effective amount of a composition comprising a TVEMP reduces or inhibits aberrant new blood vessel formation by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TVEMP reduces or inhibits aberrant new blood vessel formation by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TVEMP reduces or inhibits aberrant new blood vessel formation by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of the TVEMP is the dosage sufficient to reduce or inhibit aberrant new blood vessel formation for, e.g., at least one week, at least one month, at least two months, at least three months, at least four months, at least five months, at least six months, at least seven months, at least eight months, at least nine months, at least ten months, at least eleven months, or at least twelve months.

The actual therapeutically effective amount of a composition comprising a TVEMP to be administered to a mammal can be determined by a person of ordinary skill in the art by taking into account factors, including, without limitation, the type of aberrant new blood vessel formation, the location of the aberrant new blood vessel formation, the cause of the aberrant new blood vessel formation, the severity of the aberrant new blood vessel formation, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof. Additionally, where repeated administration of a composition comprising a TVEMP is used, the actual effect amount of a composition comprising a TVEMP will further depend upon factors, including, without limitation, the frequency of administration, the half-life of the composition comprising a TVEMP, or any combination thereof. It is known by a person of ordinary skill in the art that an effective amount of a composition comprising a TVEMP can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a composition comprising a TVEMP to a mammal, a therapeutically effective amount generally is in the range of about 1 fg to about 3.0 mg. In aspects of this embodiment, an effective amount of a composition comprising a TVEMP can be, e.g., about 100 fg to about 3.0 mg, about 100 pg to about 3.0 mg, about 100 μg to about 3.0 mg, or about 100 pg to about 3.0 mg. In other aspects of this embodiment, an effective amount of a composition comprising a TVEMP can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a composition comprising a TVEMP can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TVEMP can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

As another non-limiting example, when administering a composition comprising a TVEMP to a mammal, a therapeutically effective amount generally is in the range of about 0.00001 mg/kg to about 3.0 mg/kg. In aspects of this embodiment, an effective amount of a composition comprising a TVEMP can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a composition comprising a TVEMP can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a composition comprising a TVEMP can be, e.g., at most 0.00001 mg/kg, at most 0.0001 mg/kg, at most 0.001 mg/kg, at most 0.01 mg/kg, at most 0.1 mg/kg, or at most 1 mg/kg.

Dosing can be single dosage or cumulative (serial dosing), and can be readily determined by one skilled in the art. For instance, treatment of a disease or disorder associated with aberrant new blood vessel formation may comprise a one-time administration of an effective dose of a composition comprising a TVEMP. As a non-limiting example, an effective dose of a composition comprising a TVEMP can be administered once to a patient, e.g., as a single injection or deposition at or near the site exhibiting a symptom of aberrant new blood vessel formation. Alternatively, treatment of a cancer may comprise multiple administrations of an effective dose of a composition comprising a TVEMP carried out over a range of time periods, such as, e.g., daily, once every few days, weekly, monthly or yearly. As a non-limiting example, a composition comprising a TVEMP can be administered once or twice yearly to a mammal. The timing of administration can vary from mammal to mammal, depending upon such factors as the severity of a mammal's symptoms. For example, an effective dose of a composition comprising a TVEMP can be administered to a mammal once every three months for an indefinite period of time, or until the patient no longer requires therapy. A person of ordinary skill in the art will recognize that the condition of the mammal can be monitored throughout the course of treatment and that the effective amount of a composition comprising a TVEMP that is administered can be adjusted accordingly.

A composition comprising a TVEMP as disclosed herein can also be administered to a mammal in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A TVEMP comprising a targeting domain, a Clostridial toxin translocation domain and a Clostridial toxin enzymatic domain.
2. A TVEMP comprising a targeting domain, a Clostridial toxin translocation domain and a Clostridial toxin enzymatic domain, and an exogenous protease cleavage site.
3. The TVEMP of embodiment 1, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
4. The TVEMP of embodiment 2, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
5. The TVEMP of embodiments 1-4, wherein the targeting domain is an apelin targeting domain, an anthrax protective antigen (APA) targeting domain, an angiogenic factor with G patch and FHA domain 1 (AGGF1) targeting domain, an angiopoietin targeting domain, an angiostatin targeting domain, an angiogenin targeting domain, an endothelin targeting domain, an endothelial cell-specific molecule (ECSM) targeting domain, an ephrin targeting domain, an erythropoietin targeting domain, a hepatocyte growth factor (HGF) targeting domain, a netrin targeting domain, a brain specific angiogenesis inhibitor (BSAI) targeting domain, a Delta-like (DII) targeting domain, a jagged targeting domain, a semaphorin (Sema) targeting domain, a thrombospondin targeting domain, a chondromodulin targeting domain, a 16 kDa prolactin fragment targeting domain, a granulocyte macrophage-colony stimulating factor (GM-CSF) targeting domain, an Interferon-α (IFN-α) targeting domain, a pigment epithelium-derived factor (PEDF) targeting domain, a troponin targeting domain, a Vasohibin (VASH) targeting domain, a Urocortin (Ucn) targeting domain, a C-motif cytokine ligand targeting domain, a C—C-motif cytokine ligand targeting domain, a C—X—C-motif cytokine ligand targeting domain, and an endothelial-monocyte activating polypeptide (EMAP) targeting domain.
6. The TVEMP of embodiment 5, wherein the apelin targeting domain comprises amino acids 42-77 of SEQ ID NO: 82, SEQ ID NO: 83, SEQ ID NO: 84, SEQ ID NO: 85, SEQ ID NO: 86, SEQ ID NO: 87, SEQ ID NO: 88; the APA targeting domain comprises amino acids 30-764, amino acids 30-287, amino acids 30-196, amino acids 47-178, amino acids 204-628, amino acids 288-516, amino acids 517-624, amino acids 625-764 of SEQ ID NO: 89; or the AGGF1 targeting domain comprises amino acids 410-516 of SEQ ID NO: 90, amino acids 95-201 of SEQ ID NO: 91, amino acids 406-512 of SEQ ID NO: 92, amino acids 410-516 of SEQ ID NO: 93, amino acids 408-514 of SEQ ID NO: 94, amino acids 420-513 of SEQ ID NO: 95, amino acids 414-507 of SEQ ID NO: 96, amino acids 407-512 of SEQ ID NO: 97, or amino acids 469-574 of SEQ ID NO: 98.
7. The TVEMP of embodiment 5, wherein the angiopoietin targeting domain include an angiopoietin-1 targeting domain, an angiopoietin-2 targeting domain, an angiopoietin-3 targeting domain, or an angiopoietin-4 targeting domain.
8. The TVEMP of embodiment 7, wherein the angiopoietin targeting domain comprises amino acids 280-495 or amino acids 282-496 of SEQ ID NO: 99, SEQ ID NO: 100, or SEQ ID NO: 101; amino acids 280-492 or amino acids 281-493 of SEQ ID NO: 102, SEQ ID NO: 103, or SEQ ID NO: 104; amino acids 286-501, amino acids 239-451, or amino acids 292-507 of SEQ ID NO: 105, SEQ ID NO: 106, or SEQ ID NO: 107; or amino acids 286-501 or amino acids 292-507 of SEQ ID NO: 108 or SEQ ID NO: 109.
9. The TVEMP of embodiment 5, wherein the angiostatin targeting domain comprises amino acids 1-182, amino acids 1-262, amino acids 1-349, or amino acids 1-455 of SEQ ID NO: 110 or SEQ ID NO: 111; or the angiogenin targeting domain including amino acids 25-145 or amino acids 29-140 of SEQ ID NO: 112, SEQ ID NO: 113, SEQ ID NO: 114, SEQ ID NO: 115, or SEQ ID NO: 116.
10. The TVEMP of embodiment 5, wherein the endothelin targeting domain is an endothelin-1 targeting domain, an endothelin-2 targeting domain or an endothelin-3 targeting domain.
11. The TVEMP of embodiment 5, wherein the endothelin targeting domain including amino acids 53-73, amino acids 60-78, or amino acids 109-129 of SEQ ID NO: 117, SEQ ID NO: 118, SEQ ID NO: 119, SEQ ID NO: 120, SEQ ID NO: 121, or SEQ ID NO: 122, amino acids 49-69 or amino acids 96-111 of SEQ ID NO: 123 or SEQ ID NO: 124, amino acids 48-68 or amino acids 95-110 of SEQ ID NO: 125, amino acids 46-66 or amino acids 93-108 of SEQ ID NO: 126, amino acids 47-67 or amino acids 94-109 of SEQ ID NO: 127, amino acids 43-63 or amino acids 90-105 of SEQ ID NO: 128, amino acids 97-117 or amino acids 159-173 of SEQ ID NO: 129, SEQ ID NO: 130, SEQ ID NO: 131, or SEQ ID NO: 136, amino acids 85-114 or amino acids 150-171 of SEQ ID NO: 132, SEQ ID NO: 133, SEQ ID NO: 134, amino acids 83-103 or amino acids 144-158 of SEQ ID NO: 135, amino acids 53-73 or amino acids 115-135 of SEQ ID NO: 137, or amino acids 8-33 of SEQ ID NO 138; or the ECSM targeting domain including amino acids 22-184, amino acids 26-100, amino acids 46-100, or amino acids 72-146 of SEQ ID NO: 139, SEQ ID NO: 140, SEQ ID NO: 141, SEQ ID NO: 142, SEQ ID NO: 144, SEQ ID NO: 145, SEQ ID NO: 146, SEQ ID NO: 147; or amino acids 22-229 or amino acids 72-146 of SEQ ID NO: 143.
12. The TVEMP of embodiment 5, wherein the ephrin targeting domain comprises an ephrin-A1 targeting domain or an ephrin-B2 targeting domain.
13. The TVEMP of embodiment 12, wherein the ephrin targeting domain comprises amino acids 17-152 or amino acids 17-136 of SEQ ID NO: 148, SEQ ID NO: 149, SEQ ID NO: 150, or SEQ ID NO: 151; amino acids 34-170 of SEQ ID NO: 152 or SEQ ID NO: 153; or amino acids 30-170 of SEQ ID NO: 154.
14. The TVEMP of embodiment 5, wherein the erythropoietin targeting domain comprises amino acids 33-193 of SEQ ID NO: 155, or amino acids 26-192 of SEQ ID NO: 156 or SEQ ID NO: 157.
15. The TVEMP of embodiment 5, wherein the HGF targeting domain comprises a HGF targeting domain, a NK1 targeting domain, a NK2 targeting domain, a NK3 targeting domain, or a NK4 targeting domain.
16. The TVEMP of embodiment 15, wherein the HGF targeting domain comprises SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 171, or SEQ ID NO: 172.
17. The TVEMP of embodiment 5, wherein the netrin targeting domain is a netrin-1 targeting domain, a netrin-2 targeting domain, a netrin-3 targeting domain, a netrin-4 targeting domain, a netrin-5 targeting domain, a netrin-G1 targeting domain, and a netrin-G2 targeting domain.
18. The TVEMP of embodiment 17, wherein the netrin targeting domain comprises amino acids 26-604; 46-283, amino acids 285-328, amino acids 341-390, amino acids 403-451, or amino acids 487-601 of SEQ ID NO: 173, SEQ ID NO: 174, SEQ ID NO: 177, amino acids 26-603, amino acids 45-282, amino acids 284-327, amino acids 340-389, or amino acids 486-600 of SEQ ID NO: 175, amino acids 25-600, amino acids 46-283, amino acids 285-328, amino acids 403-451, or amino acids 487-598 of SEQ ID NO: 176, amino acids 26-605, amino acids 46-283, amino acids 285-328, amino acids 341-390, amino acids 403-451, or amino acids 487-601 of SEQ ID NO: 178, amino acids 26-606, amino acids 47-285, amino acids 287-330, amino acids 343-392, amino acids 405-453, or amino acids 489-603 of SEQ ID NO: 179; amino acids 34-253, amino acids 254-299, amino acids 373-422, or amino acids 456-562 of SEQ ID NO: 180, amino acids 15-581, amino acids 33-306, amino acids 262-305, amino acids 318-387, amino acids 380-428, or amino acids 464-563 of SEQ ID NO: 181; amino acids 28-580, amino acids 34-253, amino acids 254-299, amino acids 373-422, or amino acids 456-562 of SEQ ID NO: 182, SEQ ID NO: 183, SEQ ID NO: 184, or SEQ ID NO: 185; amino acids 19-628, amino acids 29-260, amino acids 262-321, amino acids 332-385, amino acids 395-445, or amino acids 516-626 of SEQ ID NO: 186 or SEQ ID NO: 189, amino acids 19-627, amino acids 29-260, amino acids 261-320, amino acids 332-392, amino acids 395-445, or amino acids 515-625 of SEQ ID NO: 187, amino acids 19-637, amino acids 37-269, amino acids 270-329, amino acids 341-396, amino acids 404-454, amino acids 525-635 of SEQ ID NO: 188, amino acids 19-630, amino acids 28-260, amino acids 261-320, amino acids 332-385, amino acids 394-445, amino acids 395-446, amino acids 517-626 of SEQ ID NO: 190, amino acids 19-778, amino acids 128-410, amino acids 412-470, amino acids 481-537, amino acids 545-595, amino acids 666-776 of SEQ ID NO: 191; amino acids 17-489, amino acids 211-265, amino acids 274-323, amino acids 360-475 of SEQ ID NO: 192 and SEQ ID NO: 193, amino acids 17-452, amino acids 172-216, amino acids 234-276, or amino acids 313-438 of SEQ ID NO: 194, amino acids 17-456, amino acids 172-216, amino acids 234-276, or amino acids 313-442 of SEQ ID NO: 195; amino acids 29-539, amino acids 29-510, amino acids 71-295, amino acids 297-341, or amino acids 420-467 of SEQ ID NO: 196, amino acids 29-480, amino acids 71-295, or amino acids 297-341 of SEQ ID NO: 197, amino acids 29-438, amino acids 71-295, or amino acids 297-341 of SEQ ID NO: 198, amino acids 76-123 of SEQ ID NO: 199, amino acids 20-55 of SEQ ID NO: 200, amino acids 118-153 of SEQ ID NO: 205, amino acids 41-77 of SEQ ID NO: 206, amino acids 62-97 of SEQ ID NO: 207, amino acids 19-510, amino acids 71-295, amino acids 276-341, or amino acids 420-455 of SEQ ID NO: 208, amino acids 19-483, amino acids 71-295, amino acids 297-341, or amino acids 364-411 of SEQ ID NO: 209, amino acids 19-438, amino acids 71-295, amino acids 297-341, or amino acids 364-411 of SEQ ID NO: 210, amino acids 19-480, amino acids 71-295, amino acids 297-341, or amino acids 364-411 of SEQ ID NO: 211, amino acids 19-460, amino acids 71-295, amino acids 297-341, or amino acids 364-411 of SEQ ID NO: 212, amino acids 19-364, amino acids 71-295, or amino acids 297-341 of SEQ ID NO: 213; amino acids 18-530, amino acids 18-507, amino acids 60-285, or amino acids 408-446 of SEQ ID NO: 214, SEQ ID NO: 215, or SEQ ID NO: 216.
19. The TVEMP of embodiment 5, wherein the DII targeting domain comprises a Delta-like-1 targeting domain, a Delta-like-2 targeting domain, or a Delta-like-3 targeting domain and the jagged targeting domain comprises a jagged-1 targeting domain or a jagged-2 targeting domain.
20. The TVEMP of embodiment 19, wherein the DII targeting domain comprises amino acids 159-221 of SEQ ID NO: 219 or SEQ ID NO: 220, amino acids 158-220 of SEQ ID NO: 221 or SEQ ID NO: 222, amino acids 47-618, amino acids 47-587, or amino acids 33-585 of SEQ ID NO: 223, SEQ ID NO: 224, SEQ ID NO: 225, or SEQ ID NO: 226, amino acids 27-685 or amino acids 155-217 of SEQ ID NO: 228, amino acids 27-686 or amino acids 156-218 of SEQ ID NO: 229; or the jagged targeting domain comprises amino acids 34-1218 or amino acids 167-229 of SEQ ID NO: 230, SEQ ID NO: 231, SEQ ID NO: 232, or SEQ ID NO: 233, 27-1200 or amino acids 178-240 of SEQ ID NO: 234, SEQ ID NO: 235, or SEQ ID NO: 237, or amino acids 37-99 or SEQ ID NO: 236.
21. The TVEMP of embodiment 5, wherein the Sema targeting domain comprises a semaphoring-3A targeting domain, a semaphoring-3F targeting domain, a semaphoring-4D targeting domain, or a semaphoring-5A targeting domain.
22. The TVEMP of embodiment 21, wherein the Sema targeting domain comprises amino acids 22-771, amino acids 57-498, amino acids 517-552, or amino acids 581-670 of SEQ ID NO: 238, SEQ ID NO: 239, amino acids 19-785, amino acids 57-529, amino acids 548-584, or amino acids 609-697 of SEQ ID NO: 240, amino acids, 57-498, amino acids 517-553, amino acids 517-553, or amino acids 578-666 of SEQ ID NO: 241, amino acids 22-862, amino acids 50-482, amino acids 503-554, or amino acids 559-637 of SEQ ID NO: 242, amino acids 22-738, amino acids 50-482, amino acids 503-549, or amino acids 600-679 of SEQ ID NO: 243, amino acids 24-861, amino acids 50-482, amino acids 503-550, amino acids 559-646 of SEQ ID NO: 244, amino acids, 23-1074, amino acids 58-468 or amino acids 486-533 of SEQ ID NO: 245, SEQ ID NO: 246.
23. The TVEMP of embodiment 5, wherein the thrombospondin targeting domain comprises a thrombospondin-1 targeting domain or a thrombospondin-2 targeting domain or the BSAI targeting domain comprises a BSAI-1 targeting domain and a BSAI-2 targeting domain.
24. The TVEMP of embodiment 5, wherein the thrombospondin targeting domain comprises amino acids 19-1170 of SEQ ID NO: 247, SEQ ID NO: 248, SEQ ID NO: 249, or SEQ ID NO: 250; or the BSAI targeting domain comprises amino acids 31-1584, amino acids 31-930, amino acids 359-520, amino acids 412-575, amino acids 264-314, amino acids 359-407, amino acids 412-462, amino acids 486-520, amino acids 525-575, amino acids 577-643, amino acids 653-875, or amino acids 880-938 of SEQ ID NO: 217, amino acids 21-1549, amino acids 21-920, amino acids 305-519, amino acids 413-586, amino acids 305-353, amino acids 413-463, amino acids 469-519, amino acids 522-586, or amino acids 597-857 of SEQ ID NO: 218.
25. The TVEMP of embodiment 5, wherein the chondromodulin targeting domain comprises amino acids 214-334 of SEQ ID NO: 251 or SEQ ID NO: 253, or amino acids 215-335 of SEQ ID NO: 252 or the 16 kDa prolactin fragment targeting domain comprises SEQ ID NO: 254.
26. The TVEMP of embodiment 5, wherein the GM-CSF targeting domain comprises amino acids 18-144, amino acids 18-138, or amino acids 27-138 of SEQ ID NO: 255, SEQ ID NO: 256, SEQ ID NO: 258, SEQ ID NO: 260, or SEQ ID NO: 261, amino acids 18-143, amino acids 18-137, or amino acids 30-137 of SEQ ID NO: 257, amino acids 18-141, amino acids 18-135, or amino acids 31-135 of SEQ ID NO: 259.
27. The TVEMP of embodiment 5, wherein the IFN-α targeting domain comprises amino acids 24-189 or amino acids 26-178 of SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, or SEQ ID NO: 270.
28. The TVEMP of embodiment 5, wherein the PEDF targeting domain comprises amino acids 20-418 or amino acids 40-415 of SEQ ID NO: 271, amino acids 20-417 or amino acids 38-410 of SEQ ID NO: 272, or amino acids 20-417 or amino acids 39-414 of SEQ ID NO: 273.
29. The TVEMP of embodiment 5, wherein the troponin targeting domain comprises amino acids 15-132 or amino acids 28-131 of SEQ ID NO: 274, SEQ ID NO: 275, or SEQ ID NO: 276.
30. The TVEMP of embodiment 5, wherein the VASH targeting domain including amino acids 319-365 of SEQ ID NO: 277 or SEQ ID NO: 278, or amino acids 329-375 of SEQ ID NO: 279; or the Ucn targeting domain including amino acids 20-112 or amino acids 72-109 of SEQ ID NO: 284 or SEQ ID NO: 285, or amino acids 21-123 or amino acids 83-119 of SEQ ID NO: 286.
31. The TVEMP of embodiment 5, wherein the CL targeting domain is a lymphotactin targeting domain.
32. The TVEMP of embodiment 31, wherein the CL targeting domain comprises amino acids 22-114, amino acids 22-93, or amino acids 30-93 of SEQ ID NO: 287, SEQ ID NO: 288, SEQ ID NO: 291, or SEQ ID NO: 292, amino acids 22-110, amino acids 22-88, or amino acids 26-83 of SEQ ID NO: 290, amino acids 22-97, amino acids 22-89, or amino acids 26-89 of SEQ ID NO: 293.
33. The TVEMP of embodiment 5, wherein the CCL targeting domain a CCL1 targeting domain and a CCL2 targeting domain.
34. The TVEMP of embodiment 33, wherein the CCL targeting domain comprises amino acids 24-96, amino acids 33-89, or amino acids 34-62 SEQ ID NO: 294 or SEQ ID NO: 295, or amino acids 24-148, amino acids 34-91, or amino acids 24-85 SEQ ID NO: 296 or SEQ ID NO: 297.
35. The TVEMP of embodiment 5, wherein the CXCL targeting domain a CXCL1 targeting domain, a CXCL2 targeting domain, a CXCL3 targeting domain, a CXCL4 targeting domain, a CXCL5 targeting domain, a CXCL6 targeting domain, a CXCL7 targeting domain, a CXCL8 targeting domain, a CXCL9 targeting domain, a CXCL10 targeting domain, a CXCL11 targeting domain, a CXCL12 targeting domain, a CXCL13 targeting domain, a CXCL14 targeting domain, a CXCL16 targeting domain, and an endothelial-monocyte activating polypeptide II (EMAP II) targeting domain.
36. The TVEMP of embodiment 35, wherein the CXCL targeting domain comprises amino acids 35-107 or amino acids 40-102 of SEQ ID NO: 298, SEQ ID NO: 299, SEQ ID NO: 303, SEQ ID NO: 304, SEQ ID NO: 306, SEQ ID NO: 309, or SEQ ID NO: 310, amino acids 31-104 or amino acids 37-99 of SEQ ID NO: 300, or SEQ ID NO: 305, amino acids 24-96 or amino acids 30-92 of SEQ ID NO: 301, SEQ ID NO: 302, or SEQ ID NO: 312, amino acids 30-100 or amino acids 33-95 of SEQ ID NO: 307 or SEQ ID NO: 308, amino acids 30-100 or amino acids 33-87 of SEQ ID NO: 311, amino acids 32-101 or amino acids 38-100 of SEQ ID NO: 313, amino acids 22-83 or amino acids 22-71 of SEQ ID NO: 314 or SEQ ID NO: 315, amino acids 30-105, amino acids, 33-104 or amino acids 41-77 of SEQ ID NO: 317, amino acids 37-114 or amino acids 46-107 of SEQ ID NO: 318, or SEQ ID NO: 319, amino acids 35-112 or amino acids 45-106 of SEQ ID NO: 320, amino acids 41-132 or amino acids 50-11 of SEQ ID NO: 321, amino acids 38-130 or amino acids 47-109 of SEQ ID NO: 322, amino acids 38-114 or amino acids 46-107 of SEQ ID NO: 323, or SEQ ID NO: 324, amino acids 37-112 or amino acids 45-106 of SEQ ID NO: 325, amino acids 35-128, amino acids 44-128, amino acids 44-124 or amino acids 60-120 of SEQ ID NO: 326, amino acids 40-119 or amino acids 51-113 of SEQ ID NO: 327, amino acids 34-113 or amino acids 49-111 of SEQ ID NO: 328, amino acids 32-111 or amino acids 47-109 of SEQ ID NO: 329, amino acids 21-99 or amino acids 31-94 of SEQ ID NO: 330, SEQ ID NO: 331, or SEQ ID NO: 333, amino acids 23-101 or amino acids 31-84 of SEQ ID NO: 332, amino acids 17-103 or amino acids 30-93 SEQ ID NO: 334, amino acids 23-125 or amino acids 28-86 of SEQ ID NO: 335, or SEQ ID NO: 336, amino acids 23-125 or amino acids 27-89 of SEQ ID NO: 337, amino acids 24-126 or amino acids 28-90 of SEQ ID NO: 338, amino acids 22-125 or amino acids 29-85 of SEQ ID NO: 339, or SEQ ID NO: 340, amino acids 22-98 or amino acids 27-91 of SEQ ID NO: 341, SEQ ID NO: 342, or SEQ ID NO: 344, amino acids 22-102 or amino acids 27-89 of SEQ ID NO: 343, SEQ ID NO: 345, or SEQ ID NO: 346, amino acids 22-94, amino acids 28-91, or amino acids 28-77 of SEQ ID NO: 347, SEQ ID NO: 348, SEQ ID NO: 349, SEQ ID NO: 350, SEQ ID NO: 351, or SEQ ID NO: 352, amino acids 22-89 or amino acids 27-88 of SEQ ID NO: 353, SEQ ID NO: 354, SEQ ID NO: 355, SEQ ID NO: 356, SEQ ID NO: 357, SEQ ID NO: 358, or SEQ ID NO: 359, amino acids 23-109, amino acids 29-93 or amino acids 30-92 of SEQ ID NO: 360, SEQ ID NO: 361, SEQ ID NO: 362, or SEQ ID NO: 363, amino acids 22-108 or amino acids 29-88 of SEQ ID NO: 364, amino acids 35-111 or amino acids 45-97 SEQ ID NO: 365, or SEQ ID NO: 366, amino acids 223-99 or amino acids 33-85 of SEQ ID NO: 367, SEQ ID NO: 368, SEQ ID NO: 369, SEQ ID NO: 370, or SEQ ID NO: 371, amino acids 30-254, amino acids 30-205, or amino acids 32-107 of SEQ ID NO: 372, amino acids 26-252, amino acids 27-247, or amino acids 27-198 of SEQ ID NO: 373 or SEQ ID NO: 376, amino acids 25-250 or amino acids 25-200 of SEQ ID NO: 374, amino acids 27-246 or amino acids 27-201 of SEQ ID NO: 375, amino acids 147-312 or amino acids 150-252 of SEQ ID NO: 377, amino acids 145-310 or amino acids 148-250 of SEQ ID NO: 378, or amino acids 137-299 or amino acids 137-239 of SEQ ID NO: 379.
37. The TVEMP of embodiments 1-36, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
38. The TVEMP of embodiments 1-37, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
39. The TVEMP of embodiments 2 and 4-38, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
40. A composition comprising a TVEMP of 1-39.
41. The composition of 40, wherein the composition is a pharmaceutical composition.
42. The composition of 41, wherein the pharmaceutical composition comprises a pharmaceutical carrier, pharmaceutical excipient, or any combination thereof.
43. A use of a TVEMP of embodiments 1-42 in the manufacturing a medicament for treating a disease or disorder associated with aberrant new blood vessel formation in a mammal in need thereof.
44. A method of a disease or disorder associated with aberrant new blood vessel formation in a mammal, the method comprising the step of administering to the mammal in need thereof a therapeutically effective amount of a composition including a TVEMP of embodiments 1-43, wherein administration of the composition reduces a symptom of a disease or disorder associated with aberrant new blood vessel formation.
45. A use of a TVEMP for the treatment of a disease or disorder associated with aberrant new blood vessel formation in a mammal in need thereof, the use comprising the step of administering to the mammal a therapeutically effective amount of a composition including a TVEMP of embodiments 1-43, wherein administration of the TVEMP reduces a symptom of a disease or disorder associated with aberrant new blood vessel formation.
46. The method of embodiment 44 or use of embodiment 45, wherein the disease or disorder associated with aberrant new blood vessel formation is a retinopathy, a macula degeneration, a choroidal neovascularization, an atherosclerosis, a coronary atherosclerotic plaque formation, an endometriosis, an idiopathic pulmonary fibrosis, chronic inflammatory/fibroproliferative disorder, a rheumatoid arthritis, a psoriasis, or a cancer.

### EXAMPLES

The following examples illustrate representative embodiments now contemplated, but should not be construed to limit the disclosed TVEMPs, compositions including TVEMPs, and methods of treating cancer using such compositions.

### Example 1

### Light Chain Assays

This example illustrates how to screen target cells in order to determine which Clostridial toxin light chain had an effect sufficient to provide a therapeutic benefit in a disease treatment.

To identify which Clostridial toxin light chain or active fragment thereof was useful in making a TVEMP for treating a target cell using a method disclosed herein, a Clostridial toxin light chain cleavage assay was conducted. These assays address two fundamental issues. First, the light chains of the various botulinum neurotoxin serotypes cleave different SNARE substrates. In addition, some cells may only express SNAP-23 which is not cleavable by naturally-occurring botulinum neurotoxins. These cells would not be sensitive to LC/A, but may be sensitive to LC/B and LC/C1 if they express synaptobrevin-2 (VAMP-2) and/or Syntaxin, respectively. Second, this transfection assay allows the examination of the cellular effects of the light chains on target cells in a way that is independent of receptor binding and translocation into the cell. Taken together, this assay allows the examination of the effects of cleaving SNARE proteins on a variety of target cell lines encompassing several types of human diseases.

Mammalian expression constructs encoding a fusion protein comprising a green fluorescent protein (GFP) linked to a light chain of different botulinum neurotoxin serotypes were made using standard procedures. These expression constructs were designated 1) pQBI25/GFP, a construct expressing GFP of SEQ ID NO: 201 encoded by the polynucleotide of SEQ ID NO: 202; 2) pQBI25/GFP-LC/A, a construct expressing GFP-LC/A fusion protein of SEQ ID NO: 203 encoded by the polynucleotide of SEQ ID NO: 204; 3) pQBI/GFP-LC/B, a construct expressing GFP-LC/B fusion protein of SEQ ID NO: 205 encoded by the polynucleotide of SEQ ID NO: 206; 4) pQBI/GFP-LC/C1, a construct expressing GFP-LC/C1 fusion protein of SEQ ID NO: 207 encoded by the polynucleotide of SEQ ID NO: 208; and 5) pQBI/GFP-LC/E, a construct expressing GFP-LC/E fusion protein of SEQ ID NO: 209 encoded by the polynucleotide of SEQ ID NO: 210. The light chains for these particular botulinum toxin serotypes were selected because overall, the light chains cleave one of the three predominant SNARE proteins SNAP-25, VAMP, or Syntaxin.

To culture cells, an appropriate density of cells were plated into the wells of 6-well tissue culture plates containing 3 mL of an appropriate medium (Table 5). The cells were grown in a 37° C. incubator under 5% carbon dioxide until cells reached the appropriate density (about 1×10⁶ cells). A 500 μL transfection solution was prepared by adding 250 μL of OPTI-MEM Reduced Serum Medium containing 10 μL of LipofectAmine 2000 (Invitrogen Inc., Carlsbad, Calif.), incubated at room temperature for 5 minutes, to 250 μL of OPTI-MEM Reduced Serum Medium containing 5 μg of the desired mammalian expression construct. This transfection mixture was incubated at room temperature for approximately 25 minutes. The growth media was replaced with fresh unsupplemented serum-free media and the 500 μL transfection solution was added to the cells. The cells were then incubated in a 37° C. incubator under 5% carbon dioxide for approximately 8 hours. The transfection media was replaced with fresh unsupplemented serum-free media and the cells then incubated in a 37° C. incubator under 5% carbon dioxide for approximately 48 hours. After this incubation, the cells were washed by aspirating the media and rinsing each well with 3 mL of 1×PBS.

TABLE 5
Cell Lines and Media

| Cell Line | Origin | Source | Serum Growth Media Composition |
|---|---|---|---|
| RT4 | Human urinary | ATCC HTB-2 | McCoy's 5a media with 10% fetal bovine |
| | bladder transitional | | serum, 100 U/mL Penicillin, and 100 μg/mL |
| | cell carcinoma | | Streptomycin |
| P19 | Mouse embryonic | ATCC CRL-1825 | Alpha Minimal Essential Medium media |
| | carcinoma | | with 7.5% bovine calf serum, 2.5% fetal |
| | | | bovine calf serum, 100 U/mL Penicillin, and |
| | | | 100 μg/mL Streptomycin |
| NCI H69 | Human small lung | ATCC HTB-119 | RPMI-1640 media with 10% fetal bovine |
| | carcinoma | | serum, 100 U/mL Penicillin, and 100 μg/mL |
| | | | Streptomycin |
| NCI H82 | Human small lung | ATCC HTB-175 | RPMI-1640 media with 10% fetal bovine |
| | carcinoma | | serum, 100 U/mL Penicillin, and 100 μg/mL |
| | | | Streptomycin |
| DU-145 | Human prostate | ATCC HTB-81 | Eagle's Minimum Essential Medium with 10% |
| | carcinoma derived | | fetal bovine serum, 100 U/mL Penicillin, |
| | from brain | | and 100 μg/mL Streptomycin |
| T24 | Human urinary | ATCC HTB-4 | McCoy's 5a media with 10% fetal bovine |
| | bladder transitional | | serum, 100 U/mL Penicillin, and 100 μg/mL |
| | cell carcinoma | | Streptomycin |
| J82 | Human urinary | ATCC HTB-1 | Eagle's Minimum Essential Medium with 10% |
| | bladder transitional | | fetal bovine serum, 100 U/mL Penicillin, |
| | cell carcinoma | | and 100 μg/mL Streptomycin |
| HIT-T15 | Syrian Golden | ATCC CRL-1777 | Eagle's Minimum Essential Medium (low |
| | Hamster, pancreatic | | glucose) with 10% fetal bovine serum, 100 U/mL |
| | islet of Langerhans | | Penicillin, and 100 μg/mL |
| | beta cells | | Streptomycin |
| ARPE-19 | Human Retinal | ATCC: CRL-2302 | DMEM: F12 media with 10% fetal bovine |
| | Epithelial Cells | | serum, 100 U/ml Penicillin, and 100 μg/ml |
| | | | Streptomycin |
| RF/6A | Macaca Choroid | ATCC: CRL-1780 | EMEM media with 10% fetal bovine serum, |
| | Retinal Endothelial | | 100 U/ml Penicillin, and 100 μg/ml |
| | Cells | | Streptomycin |

The cells were first analyzed using fluorescent microscopy for the expression of GFP, which also indicated the simultaneous expression of the attached light chain. To detect the expression and subcellular localization of the GFP-LC fusion proteins, the cells were examined by confocal microscopy. Cells from the cell lines RT4, P19, NCl H69, NCl H82, DU145, T24, ARPE-19, RF/6A, and J82, transfected and washed as described above, were fixed with 4% paraformaldehyde. The fixed cells were imaged with a confocal microscope using a 488 nm excitation laser and an emission path of 510-530 nm. The data shows that each cell type was successfully transfected and, that except the small cell lung cancer cell lines NCl H69 and NCl H82, cells from each cell line expressed both GFP and the GFP-light chain fusion proteins (Table 6).

TABLE 6
Expression of Mammalian Constructs in Cells

| | | Expression | | | | |
|---|---|---|---|---|---|---|
| | | | GFP- | GFP- | GFP- | |
| Cell Line | Origin | GFP | LC/A | LC/B | LC/C1 | GFP-LC/E |
| RT4 | Bladder | + | + | + | + | + |
| | carcinoma | | | | | |
| P19 | Embryonic | + | + | + | + | + |
| | carcinoma | | | | | |
| NCI H69 | Small Cell Lung | − | − | − | − | − |
| | carcinoma | | | | | |
| NCI H82 | Small Cell Lung | − | − | − | − | − |
| | carcinoma | | | | | |
| DU145 | Prostate | + | + | + | + | + |
| | carcinoma | | | | | |
| T24 | Bladder | + | + | + | + | + |
| | carcinoma | | | | | |
| J82 | Bladder | + | + | + | + | + |
| | carcinoma | | | | | |
| ARPE-19 | Retinal Epithelial | + | + | +/− | + | − |
| | pigment | | | | | |
| RF/6A | Retinal | + | + | + | + | + |
| | Endothelial | | | | | |

In order for cancer cells to be sensitive to the endoproteolytic cleavage, the target SNARE protein must be endogenously expressed and accessible to the light chain cleavage. To detect the presence of cleaved SNARE products a Western blot analysis was performed. Cells from the cell lines RT4, P19, NCl H69, NCl H82, DU145, T24, ARPE-19, RF/6A, and J82, transfected and washed as described above, were lysed, by adding 200 μL of 2×SDS-PAGE Loading Buffer to each well, and the lysates were transferred to tubes and heated to 95° C. for 5 minutes. A 12 μL of each sample was separated by MOPS polyacrylamide gel electrophoresis using NuPAGE® Novex 4-12% Bis-Tris precast polyacrylamide gels (Invitrogen Inc., Carlsbad, Calif.) under denaturing, reducing conditions. Separated peptides were transferred from the gel onto nitrocellulose membranes by Western blotting using an electrophoretic tank transfer apparatus. The membranes were blocked by incubation, at room temperature, for 1 hour with gentle agitation, in a Blocking Solution containing Tris-Buffered Saline (TBS) (25 mM 2-amino-2-hydroxymethyl-1,3-propanediol hydrochloric acid (Tris-HCl)(pH 7.4), 137 mM sodium chloride, 2.7 mM potassium chloride), 0.1% polyoxyethylene (20) sorbitan monolaureate, 2% Bovine Serum Albumin (BSA), and 5% nonfat dry milk. Blocked membranes were incubated at 4° C. over night in TBS, 0.1% polyoxyethylene (20) sorbitan monolaureate, 2% BSA, and either 1) a 1:5,000 dilution of S9684 α-SNAP-25 rabbit polyclonal antiserum as the primary antibody (Sigma, St. Louis, Mo.); 2) a 1:5,000 dilution of sc17836 α-Syntaxin-1 rabbit polyclonal antiserum as the primary antibody (Santa Cruz Biotechnology, Santa Cruz, Calif.); or 3) a 1:5,000 dilution of sc69706 a-VAMP-2 mouse polyclonal antiserum as the primary antibody (Santa Cruz Biotechnology, Santa Cruz, Calif.). Primary antibody probed blots were washed three times for 5 minutes each time in TBS, polyoxyethylene (20) sorbitan monolaureate. Washed membranes were incubated at room temperature for 1 hour in TBS, 0.1% polyoxyethylene (20) sorbitan monolaureate, 2% BSA containing either 1) a 1:5,000 dilution of 81-6720 goat polyclonal α-mouse immunoglobulin G, heavy and light chains (IgG, H+L) antibody conjugated to horseradish peroxidase (Invitrogen, Inc., Carlsbad, Calif.) as a secondary antibody; or 2) a 1:5,000 dilution of 81-6120 goat polyclonal α-rabbit immunoglobulin G, heavy and light chains (IgG, H+L) antibody conjugated to horseradish peroxidase (Invitrogen, Inc., Carlsbad, Calif.) as a secondary antibody. Secondary antibody-probed blots were washed three times for 5 minutes each time in TBS, 0.1% polyoxyethylene (20) sorbitan monolaureate. Signal detection of the labeled SNARE products were visualized using the ECL Plus™ Western Blot Detection System, a chemiluminescence-based detection system, (GE Healthcare-Amersham, Piscataway, N.J.). The membranes were imaged and the percent of cleaved SNARE product were quantified with a Typhoon 9410 Variable Mode Imager and Imager Analysis software (GE Healthcare-Amersham, Piscataway, N.J.). The data shows that SNAP-25 and VAMP-2 were expressed in some cell types, while Syntaxin was expressed in each cell type tested (Table 7).

TABLE 7
Presence of SNARE in Cells

| | | SNARE Presence in Cells | | |
|---|---|---|---|---|
| Cell Line | Origin | SNAP-25 | VAMP-2 | Syntaxin-1 |
| RT4 | Bladder | − | + | + |
| | carcinoma | | | |
| P19 | Embryonic | + | − | + |
| | carcinoma | | | |
| NCI H69 | Small cell Lung | ND | ND | ND |
| | carcinoma | | | |
| NCI H82 | Small cell Lung | ND | ND | ND |
| | carcinoma | | | |
| DU145 | Prostate | + | + | + |
| | carcinoma | | | |
| T24 | Bladder | − | + | + |
| | carcinoma | | | |
| J82 | Bladder | + | − | + |
| | carcinoma | | | |
| ARPE-19 | Retinal Epithelial | − | − | + |
| | pigment | | | |
| RF/6A | Retinal | + | − | + |
| | Endothelial | | | |

In addition, the data shows that 1) BoNT/A light chain was able to cleave SNAP-25 present in cells from a P19 embryonic carcinoma cell line, a DU145 prostate carcinoma cell line, RF/6A retinal endothelial and a J82 urinary bladder carcinoma cell line (Table 8); 2) BoNT/E light chain was able to cleave SNAP-25 present in cells from a P19 embryonic carcinoma cell line, RF/6A retinal endothelial, and a J82 urinary bladder carcinoma cell line (Table 8); 3) BoNT/B light chain was unable to cleave VAMP-2 in all cell lines tested except ARPE-19 and RF/6A that do not express VAMP-2 (Table 8); and 4) BoNT/C1 light chain was able to cleave Syntaxin-1 present in cells from a T24 urinary bladder carcinoma cell line, ARPE-19 retinal epithelial pigment cells, and RF/6A retinal endothelial cells (Table 8). These results indicate that treatment of target cells with the appropriate Clostridial toxin light chain will cleave one of three SNARE proteins to inhibit exocytosis. This inhibition will prevent the release of growth factors, angiogenic factors, proteases, and anti-apoptotic survival factors necessary for cancer cell growth and survival. Moreover, this inhibition will inhibit the delivery of receptors involved in proliferation, migration, survival, and chemotaxis to the surface of cells involved in aberrant blood vessel formation.

TABLE 8
Cleavage of SNARE by Light Chain

| | | SNARE Cleavage by Light Chain | | | |
|---|---|---|---|---|---|
| | | SNAP-25 | | VAMP-2 | Syntaxin-1 |
| Cell Line | Origin | LC/A | LC/E | LC/B | LC/C1 |
| RT4 | Bladder | − | − | − | − |
| | carcinoma | | | | |
| P19 | Embryonic | + | + | − | − |
| | carcinoma | | | | |
| NCI H69 | Small Cell Lung | ND | ND | ND | ND |
| | carcinoma | | | | |
| NCI H82 | Small Cell Lung | ND | ND | ND | ND |
| | carcinoma | | | | |
| DU145 | Prostate | + | − | − | − |
| | carcinoma | | | | |
| T24 | Bladder | − | − | − | + |
| | carcinoma | | | | |
| J82 | Bladder | + | + | − | − |
| | carcinoma | | | | |
| ARPE-19 | Retinal Epithelial | − | − | − | + |
| | pigment | | | | |
| RF/6A | Retinal | + | + | − | + |
| | Endothelial | | | | |

To further test whether SNARE cleavage disrupts exocytosis of hormones and growth factors, an insulin release assay was performed. HIT-T15 cells release insulin when placed in high concentration of glucose. It has also been shown these cells express SNAP-25, and that SNAP-25 is an integral component of the SNARE complex needed for insulin release. HIT-T15 cells, transfected and washed as described above, were placed in DMEM media containing either 1) 5.6 mM glucose for basal insulin release (low glucose); or 2) 25.2 mM glucose for evoked insulin release (high glucose). Cells were incubated in a 37° C. incubator under 5% carbon dioxide for approximately 1 hour to allow for insulin release. The incubated media was collected and the amount of insulin released was determined using an insulin ELISA kit. The assay was performed according to the manufacturer's instructions (APLCO Diagnostics, Salem, N.H.). Exocytosis was expressed as the amount of insulin released per 1×10⁶ cells per hour.

The data shows that HIT-T15 cells transfected with GFP-LC/A, GFP-LC/B, and GFP-LC/E released less insulin than untransfected cells or cells transfected with GFP (Table 9). In addition, the basal insulin released in media containing a low glucose concentration (5.6 mM) remained unchanged between the transfected cells. The data indicate that BoNT/A, BoNT/B and BoNT/E light chains inhibited the release of insulin by cleaving SNAP-25 or VAMP-2 in HIT-T15 cells.

TABLE 9
Insulin Release from HIT-H15 Cells

| Construct | 5.6 mM Glucose (Low) | 25.2 mM Glucose (High) |
|---|---|---|
| Untransfected | 6.5 +/− 0.1 | 9.9 +/− 2.9 |
| Control | | |
| GFP | 4.3 +/− 0.7 | 10.8 +/− 2.1 |
| GFP-LCA | 3.2 +/− 0.4 | 4.5 +/− 0.6 |
| GFP-LCB | 3.4 +/− 0.2 | 5.5 +/− 0.9 |
| GFP-LCE | 4.2 +/− 0.7 | 4.4 +/− 1.0 |

The botulinum toxin light chain activity may also inhibit the trafficking of proteins to and from the plasma membrane. To test whether SNARE cleavage disrupts delivery and localization of receptors to the plasma membrane, the presence or absence of cell membrane proteins was determined in cells transfected with botulinum toxin light chains. Cells from the cell lines DU145, J82, ARPE-19, and PF/6A transfected and washed as described above, were treated with 2 mM NHS-LC-Biotin (Thermo Scientific, Rockford, Ill.) at 4° C. for 2 hours. The cells were then treated with 250 mM Tris-HCl (pH 7.5) for 30 minutes at 4° C., and then washed three times in TBS. Membranes proteins were isolated using the Membrane Protein extraction kit (Calbiochem, San Diego, Calif.) according to the manufacturer's instructions. The biotinylated proteins were precipitated with immobilized-avidin (Thermo Scientific, Rockford, Ill.). After three washes with TBS, the samples were suspended in 50 μL 2×SDS-PAGE loading buffer and separated by MOPS polyacrylamide gel electrophoresis using NuPAGE® Novex 4-12% Bis-Tris precast polyacrylamide gels (Invitrogen Inc., Carlsbad, Calif.) under denaturing, reducing conditions. The gel was washed and fixed in 10% methanol and 7% acetic acid for 30 minutes. The wash solution was removed and the gel incubated in SYPRO® Ruby protein gel stain solution (Bio-Rad Laboratories, Hercules, Calif.) for 3 hours to overnight at room temperature. The stained gel was destained in 10% methanol and 7% acetic acid for 30 minutes. Chemiluminescence from the destained gel was visualized with a Typhoon 9410 Variable Mode Imager and Imager Analysis software (GE Healthcare-Amersham, Piscataway, N.J.). The data show that treatment with a BoNT/A light chain inhibits the trafficking of proteins to and from the plasma membrane, which would necessarily affect the population of receptors located on the surface of the cell. This disrupted trafficking may cause the target cells to become more sensitive to apoptotic factors and less sensitive to growth signals and angiogenic factors.

By establishing the SNARE cleavage effects by the light chains, and which light chains cleaved which SNARE proteins in each cell line, TVEMPs were subsequently designed in a manner that targeted the TVEMP to receptors that were overexpressed or uniquely expressed in target cells in order to deliver the catalytic light chain.

### Example 2

### Presence of Receptor and Target in Disease Relevant Cells

This example illustrates how to determine the presence of a cognate receptor that can bind with the targeting moiety of a TVEMP disclosed herein as well as the presence of the target SNARE protein of the enzymatic domain of a TVEMP disclosed herein.

In order for a TVEMP to be an effective agent for the methods of treating cancer disclosed herein, the cancer cells must express the appropriate receptor that can bind with the targeting moiety of a TVEMP as well as the appropriate SNARE protein that can be cleaved by the enzymatic domain of the TVEMP.

To culture cells, an appropriate density of cells were plated into the wells of 96-well tissue culture plates containing 100 μL of an appropriate medium (Table 10), but without serum, and with or without 25 pg/mL of GT1b (Alexis Biochemicals, San Diego, Calif.). Cells were plated and incubated in a 37° C. incubator under 5% carbon dioxide until the cells differentiated, as assessed by standard and routine morphological criteria, such as growth arrest (approximately 3 days). The media was aspirated from each well and replaced with 100 μL of fresh media containing various concentrations of the botulinum toxin or TVEMP being tested in order to generate a full dose-response. The assay was done in triplicate. After 24 hrs treatment, the cells were washed, incubated for an additional two days without toxin or TVEMP to allow for the cleavage of the SNARE substrate. After this incubation, the cells were washed by aspirating the media and rinsing each well with 3 mL of 1×PBS. The cells were harvested by lysing in freshly prepared Lysis Buffer (50 mM HEPES, 150 mM NaCl, 1.5 mM MgCl₂, 1 mM EGTA, 1%, 4-octylphenol polyethoxylate) at 4° C. for 30 minutes with constant agitation. Lysed cells were centrifuged at 4000 rpm for 20 min at 4° C. to eliminate debris using a bench-top centrifuge. The total protein concentrations of the cell lysates were measured by Bradford assay.

TABLE 10
Cell Lines and Media

| Cell Line | Origin | Source | Serum Growth Media Composition |
|---|---|---|---|
| RT4 | Human urinary | ATCC HTB-2 | McCoy's 5a media with 10% fetal bovine |
| | bladder transitional | | serum, 100 U/mL Penicillin, and 100 μg/mL |
| | cell carcinoma | | Streptomycin |
| P19 | Mouse embryonic | ATCC CRL-1825 | Alpha Minimal Essential Medium media |
| | carcinoma | | with 7.5% bovine calf serum, 2.5% fetal |
| | | | bovine calf serum, 100 U/mL Penicillin, and |
| | | | 100 μg/mL Streptomycin |
| NCI H69 | Human small lung | ATCC HTB-119 | RPMI-1640 media with 10% fetal bovine |
| | carcinoma | | serum, 100 U/mL Penicillin, and 100 μg/mL |
| | | | Streptomycin |
| NCI H82 | Human small lung | ATCC HTB-175 | RPMI-1640 media with 10% fetal bovine |
| | carcinoma | | serum, 100 U/mL Penicillin, and 100 μg/mL |
| | | | Streptomycin |
| DU-145 | Human prostate | ATCC HTB-81 | Eagle's Minimum Essential Medium with 10% |
| | carcinoma derived | | fetal bovine serum, 100 U/mL Penicillin, |
| | from brain | | and 100 μg/mL Streptomycin |
| PC-3 | Human prostate | ATCC CRL-1435 | F-12K media with 10% fetal bovine serum, |
| | carcinoma derived | | 100 U/mL Penicillin, and 100 μg/mL |
| | from brain | | Streptomycin |
| LNCaP clone | Human prostate | ATCC CRL-1740 | RPMI-1640 Eagle's with 10% fetal bovine |
| FGC | carcinoma derived | | serum, 100 U/mL Penicillin, and 100 μg/mL |
| | from brain | | Streptomycin |
| RWPE-1 | Human prostate | ATCC CRL-11609 | Dulbecco's Minimum Essential Medium with |
| | | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| T24 | Human urinary | ATCC HTB-4 | McCoy's 5a media with 10% fetal bovine |
| | bladder transitional | | serum, 100 U/mL Penicillin, and 100 μg/mL |
| | cell carcinoma | | Streptomycin |
| J82 | Human urinary | ATCC HTB-1 | Eagle's Minimum Essential Medium with 10% |
| | bladder transitional | | fetal bovine serum, 100 U/mL Penicillin, |
| | cell carcinoma | | and 100 μg/mL Streptomycin |
| MCF-7 | Human breast | ATCC HTB-22 | Dulbecco's Minimum Essential Medium with |
| | carcinoma | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| SiMa | Human | DSMZ ACC 164 | RPMI 1640 with 10% Fetal Bovine Serum, |
| | neuroblastoma | | 0.1 mM Non-Essential Amino-Acids, 10 mM |
| | | | HEPES, 1 mM Sodium Pyruvate, 100 U/mL |
| | | | Penicillin, and 100 μg/mL Streptomycin, |
| 266.6 | Mouse pancreatic | ATCC CRL-2151 | Dulbecco's Minimum Essential Medium with |
| | | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| HIT-T15 | Hamster pancreatic | ATCC CRL-1777 | Eagle's Minimum Essential Medium (low |
| | islet of Langerhans | | glucose) with 10% fetal bovine serum, 100 U/mL |
| | beta cells | | Penicillin, and 100 μg/mL |
| | | | Streptomycin |
| HUVEC | Human Umbilical | Cell Applications, Inc., | Endothelial Cell Growth Medium (Cell |
| | Vein Endothelial | San Diego, CA, Cat. | Applications, Inc., San Diego, CA, Cat. No. |
| | Cells | No. 200-05n | 211-500) |
| ARPE-19 | Human Retinal | ATCC: CRL-2302 | DMEM: F12 media with 10% fetal bovine |
| | Epithelial Cells | | serum, 100 U/ml Penicillin, and 100 μg/ml |
| | | | Streptomycin |
| RF/6A | Macaca Choroid | ATCC: CRL-1780 | EMEM media with 10% fetal bovine serum, |
| | Retinal Endothelial | | 100 U/ml Penicillin, and 100 μg/ml |
| | Cells | | Streptomycin |

To determine whether a target cell expresses the appropriate receptor and target SNARE protein, a Western blot analysis can be performed.

In one experiment, cells from the cell lines RT4, P19, NCl H69, NCl H82, DU-145, T24, J82, LNCaP, ARPE-19, RF/6A and PC-3, transfected and washed as described above, were harvested by adding 40 μL of 2×SDS-PAGE Loading Buffer (Invitrogen, Inc., Carlsbad, Calif.) and heating the plate to 95° C. for 5 min. A 12 μL of the harvested sample was separated by MOPS polyacrylamide gel electrophoresis under denaturing, reducing conditions using 1) CRITERION® 12% Bis-Tris precast polyacrylamide gels (Bio-Rad Laboratories, Hercules, Calif.), when separating the SNAP-25₁₉₇ cleavage product; 2) NuPAGE® 12% Bis-Tris precast polyacrylamide gels (Invitrogen Inc., Carlsbad, Calif.), when separating both the uncleaved SNAP-25₂₀₆ substrate and the SNAP-25₁₉₇ cleavage product; or 3) NuPAGE® Novex 4-12% Bis-Tris precast polyacrylamide gels (Invitrogen Inc., Carlsbad, Calif.), when separating all other proteins. Separated peptides were transferred from the gel onto nitrocellulose membranes by Western blotting using a electrophoretic tank transfer apparatus. The membranes were blocked by incubation at room temperature for 1 hour with gentle agitation in a Blocking Solution containing Tris-Buffered Saline (TBS) (25 mM 2-amino-2-hydroxymethyl-1,3-propanediol hydrochloric acid (Tris-HCl)(pH 7.4), 137 mM sodium chloride, 2.7 mM potassium chloride), 0.1% polyoxyethylene (20) sorbitan monolaureate, 2% Bovine Serum Albumin (BSA), and 5% nonfat dry milk. Blocked membranes were incubated at 4° C. overnight in TBS, 0.1% polyoxyethylene (20) sorbitan monolaureate, 2% BSA, and either 1) a 1:5,000 dilution of S9684 α-SNAP-25 rabbit polyclonal antiserum as the primary antibody (Sigma, St. Louis, Mo.); 2) a 1:5,000 dilution of sc123 α-Syntaxin-1 rabbit polyclonal antiserum as the primary antibody (Santa Cruz Biotechnology, Santa Cruz, Calif.); 3) a 1:5,000 dilution of sc13992 α-VAMP-1/2/3 rabbit polyclonal antiserum as the primary antibody (Santa Cruz Biotechnology, Santa Cruz, Calif.); 4) a 1:5,000 dilution of sc50371 α-SNAP-23 rabbit polyclonal antiserum as the primary antibody (Santa Cruz Biotechnology, Santa Cruz, Calif.); 5) a 1:5,000 dilution of sc28955 α-SVC2 rabbit polyclonal antiserum as the primary antibody (Santa Cruz Biotechnology, Santa Cruz, Calif.); 6) a 1:5,000 dilution of sc123 α-FGFR3 rabbit polyclonal antiserum as the primary antibody (Santa Cruz Biotechnology, Santa Cruz, Calif.); 7) a 1:5,000 dilution of sc9112 α-KOR1 rabbit polyclonal antiserum as the primary antibody (Santa Cruz Biotechnology, Santa Cruz, Calif.); 8) a 1:5,000 dilution of H00004987-D01P α-OPRL1 rabbit polyclonal antiserum as the primary antibody (Novus Biologicals, Littleton, Colo.); and 9) a 1:5,000 dilution of sc47778 α-β-actin mouse monoclonal antiserum as the primary antibody (Santa Cruz Biotechnology, Santa Cruz, Calif.). Primary antibody probed blots were washed three times for 5 minutes each time in TBS, polyoxyethylene (20) sorbitan monolaureate. Washed membranes were incubated at room temperature for 1 hour in TBS, 0.1% polyoxyethylene (20) sorbitan monolaureate, 2% BSA containing either 1) a 1:5,000 dilution of 81-6720 goat polyclonal a-mouse immunoglobulin G, heavy and light chains (IgG, H+L) antibody conjugated to horseradish peroxidase (Invitrogen, Inc., Carlsbad, Calif.) as a secondary antibody; or 2) a 1:5,000 dilution of 81-6120 goat polyclonal a-rabbit immunoglobulin G, heavy and light chains (IgG, H+L) antibody conjugated to horseradish peroxidase (Invitrogen, Inc., Carlsbad, Calif.) as a secondary antibody. Secondary antibody-probed blots were washed three times for 5 minutes each time in TBS, 0.1% polyoxyethylene (20) sorbitan monolaureate. Signal detection of the labeled SNARE products were visualized using the ECL Plus™ Western Blot Detection System, a chemiluminescence-based detection system (GE Healthcare-Amersham, Piscataway, N.J.). The membranes were imaged and the percent of cleaved SNARE product was quantified with a Typhoon 9410 Variable Mode Imager and Imager Analysis software (GE Healthcare-Amersham, Piscataway, N.J.). The data shows that this approach can identify the receptors and SNARE proteins present in the cells comprising each cell line (Table 11).

TABLE 11
Expression of Receptors and SNARE Proteins in Target Cells

| | Expression | | | | | | | |
|---|---|---|---|---|---|---|---|---|
| Cell Line | SNAP-25 | SNAP-23 | VAMP-2 | Syntaxin-1 | FGFR3 | SV2C | OPRL-1 | KOR-1 |
| RT4 | + | − | + | + | + | + | ND | + |
| P19 | + | − | − | + | + | − | ND | + |
| NCI H69 | + | − | + | + | + | − | ND | + |
| NCI H82 | + | − | + | + | + | − | ND | + |
| DU-145 | ++ | + | ++ | ++ | +++ | ND | ND | + |
| PC-3 | − | ++ | +/− | ++ | +++ | ND | ND | + |
| LNCaP | + | + | + | + | +++ | +++ | ++ | + |
| clone FGC | | | | | | | | |
| T24 | − | ++ | + | + | ++ | ++ | ++ | + |
| J82 | ++ | +/− | ++ | + | +++ | ++ | ++ | + |
| ARPE-19 | − | + | − | + | + | + | − | + |
| RF/6A | + | − | − | + | + | + | − | + |

ND, not determined

Once cell lines comprising cells including the appropriate receptor and SNARE proteins were identified, the ability of a botulinum toxin or TVEMP to intoxicate these cells can be determined by detecting the presence of cleaved SNARE products using Western blot analysis. An appropriate density of cells from each cell line to be tested are plated into the wells of 96-well tissue culture plates containing 100 μL of an appropriate medium (Table 7) with or without 25 μg/mL of GT1b (Alexis Biochemicals, San Diego, Calif.). Cells are plated and incubated in a 37° C. incubator under 5% carbon dioxide until the cells differentiated, as assessed by standard and routine morphological criteria, such as growth arrest (approximately 3 days). The media is aspirated from each well and is replaced with 100 μL of fresh media containing various concentrations of the botulinum toxin or TVEMP being tested sufficient to generate a full dose-response. The assay is done in triplicate. After 24 hrs treatment, the cells are washed, incubated for an additional two days without toxin or TVEMP to allow for the cleavage of the SNARE substrate. After this incubation, the cells are washed by aspirating the media and rinsing each well with 3 mL of 1×PBS. The cells are harvested by lysing in freshly prepared Lysis Buffer (50 mM HEPES, 150 mM NaCl, 1.5 mM MgCl₂, 1 mM EGTA, 1%, 4-octylphenol polyethoxylate) at 4° C. for 30 minutes with constant agitation. Lysed cells are centrifuged at 4000 rpm for 20 min at 4° C. to eliminate debris using a bench-top centrifuge. The protein concentrations of cell lysates are measured by Bradford assay. Samples of the cell lysates are analyzed by Western blot analysis as described above.

In one experiment, differentiated cells from the cell lines LNCaP, J82, and MCF-7, transfected as described above. The media was aspirated from each well and the differentiated cells were treated by replacing with fresh media containing either 1) 0 (untreated sample), 0.12 nM, 0.36 nM, 1.1 nM, 3.3 nM, 10 nM, 30 nM, and 90 nM of a BoNT/A; 2) 0 (untreated sample), and 50 nM of a BoNT/A; 3) 0 (untreated sample), 0.12 nM, 0.36 nM, 1.1 nM, 3.3 nM, 10 nM, 30 nM, and 90 nM of a TVEMP designated Noci-LH_{N}/A; or 4) 0 (untreated sample), and 166 nM of a TVEMP designated Noci-LHN/A. After 1) 3-15 hours; 2) 6 hours or 3) 24 hours treatment, the cells were washed, incubated for an additional 16 hours without toxin or TVEMP to allow for the cleavage of the SNAP-25 substrate. After this incubation, the cells were washed and harvested as described above. The presence of cleaved SNAP-25 product was detected using Western blot analysis as described above using a 1:5,000 dilution of S9684 a-SNAP-25 rabbit polyclonal antiserum as the primary antibody (Sigma, St. Louis, Mo.) as the primary antibody and a 1:5,000 dilution of 81-6120 goat polyclonal a-rabbit immunoglobulin G, heavy and light chains (IgG, H+L) antibody conjugated to horseradish peroxidase (Invitrogen, Inc., Carlsbad, Calif.) as a secondary antibody. These results are shown in Table 12.

TABLE 12
Cleavage of SNARE Substrate

Lowest Concentration and Earliest Time
for Cleavage Detection

| Cell Line | BoNT/A | Noci-LH_{N}/A |
|---|---|---|
| LNCaP | 50 nM at 9 hours | 166 nM at 9 hours |
| J82 | 50 nM at 3 hours | 166 nM at 3 hours |
| | 1.1 nM at 24 hours | |
| MCF-7 | 1.1 nM at 6 hours | ND |

ND, not determined

Taken together, the data shows that 1) BoNT/A was able to cleave SNAP-25 present in cells from a LNCaP prostate carcinoma cell line, a J82 urinary bladder carcinoma cell line, and a MCF-7 breast carcinoma cell line (Table 12); 2) Noci-LH_{N}/A was able to cleave SNAP-25 present in cells from a LNCaP prostate carcinoma cell line and a J82 urinary bladder carcinoma cell line (Table 12). These results indicate that treatment of cancer cells with the appropriate Clostridial toxin light chain will cleave one of three SNARE proteins to inhibit exocytosis. This inhibition will prevent the release of growth factors, angiogenic factors, and anti-apoptotic survival factors necessary for cancer cell growth and survival. Lastly, these experiments illustrate the validity of the general concept that intracellular delivery of a botulinum light chain into cancer cells results in cleavage of the appropriate SNARE protein not only by transfecting light chain constructs, but also by using the endogenous signal transduction pathway for the targeting domain.

### Example 3

### Effects of Light Chain Delivery on Angiogenesis

This example illustrates that treatment with a botulinum toxin or TVEMP will affect angiogenesis to a degree sufficient to provide a therapeutic benefit to an individual suffering from a disease or disorder associated with aberrant new vessel formation.

The blockade of exocytosis resulting from a treatment with botulinum toxin or TVEMP based on LHN/A-G will likely prevent the release of angiogenic factors, including, e.g., Vascular endothelial growth factor (VEGF), Fibroblast Growth Factor-1 (FGF1) and FGF2. Preventing the release of these angiogenic factors will reduce, or altogether inhibit, angiogenesis in the area where the toxin or TVEMP is administered. To test whether such a treatment reduces or inhibits angiogenesis, four different assays were performed: a VEGF release assay, a cell migration assay, an in vitro blood vessel formation assay, and a human angiogenesis protein array assay.

VEGF is known to be a potent mitogen for vascular endothelial cells and an inducer of physiological and pathological angiogenesis. To validate the potential for a botulinum toxin or TVEMP in inhibiting angiogenesis, the ability of a toxin or TVEMP to inhibit release of VEGF from a cell was assessed. To conduct a VEGF release assay, about 600,000 cells from a SiMa cell line were plated into the wells of 6-well collagen IV tissue culture plates containing 3 mL of a serum-free medium containing Minimum Essential Medium, 2 mM GlutaMAX™ I with Earle's salts, 1×B27 supplement, 1×N2 supplement, 0.1 mM Non-Essential Amino Acids, 10 mM HEPES and 25 μg/mL GT1b. These cells were incubated in a 37° C. incubator under 5% carbon dioxide until the cells differentiated, as assessed by standard and routine morphological criteria, such as growth arrest and neurite extension (approximately 3 days). The media from the differentiated cells was aspirated from each well and replaced with fresh media containing either 0.77 mg/mL of a BoNT/A or 1 mg/mL of a Noci-LH_{N}/A TVEMP. As a control, cells were treated with media alone in parallel. After treatment the media was removed and replaced with fresh differentiation media. A 60 μL aliquot of media was removed from each well and replaced with 100 μL differentiation media 1 day, 2 days, 3 days, and 4 days after the addition of fresh differentiation media. The removed media was stored at −20° C. until needed. After the last sample was removed, the cells were trypsinized and the number of cells in each well was counted.

The presence of VEGF in the collected samples was detected using a K151BMB-1 VEGF tissue culture assay (Meso Scale Discovery, Gaithersburg, Md.). A MULTI-ARRAY® 96-well Small Spot Plate VEGF plate was blocked with 150 μL Blocking Buffer (PBS with 0.05% polyoxyethylene (20) sorbitan monolaureate, 2% ECL Blocking reagent (GE Healthcare-Amersham, Piscataway, N.J.), and 1% goat serum (Rockland Immunochemicals, Gilbertsville, Pa.) and shaken at 600 rpm for one hour. The blocking buffer was discharged and 25 μL of each sample was added to each well of the VEGF plate and the plate was incubated at 4° C. for 2 hours. The plate was washed three times with 200 μL PBS-T (PBS plus 0.05% Tween-20) and then 25 μl of SULFO-TAG α-hVEGF mouse monoclonal antibody 5 pg/mL in 2% antibody buffer (PBS plus 0.05% polyoxyethylene (20) sorbitan monolaureate, and 2% ECL Blocking reagent (GE Healthcare-Amersham, Piscataway, N.J.) added and incubated on a shaker at 600 rpm at RT for 1 hour. Plates were washed three times with PBS-T and then 150 μL Read Buffer (MSD, Cat# R92TC-1) were added per well. Plates were read in a SECTOR™ Imager 6000 Image Reader (Meso Scale Discovery, Gaithersburg, Md.). The data was then exported into Microsoft Office Excel 2007. The amount of VEGF detected was normalized to the number of cells present in the well and the percent VEGF release value was calculated using the control as the 100% value.

The data shows that treatment with BoNT/A inhibits VEGF release by about 50% in SiMa cells (Table 13). Although the addition of Noci-LH_{N}/A TVEMP did not appear to inhibit VEGF release, this result could be due to the lower potency of Noci-LH_{N}/A TVEMP compared to BoNT/A in SiMa cells. The EC₅₀ of BoNT/A in differentiated SiMa cells is less than about 0.5 nM, while the EC₅₀ of Noci-LH_{N}/A TVEMP is more than 30 nM. As such, the lack of effect of Noci-LH_{N}/A TVEMP in SiMa cells is simply due to the low amount of OPRL-1 receptor present in these cells. This lack of effect corroborates the concept that cells expressing low levels of the targeted receptor will not be affected by botulinum toxin or TVEMP treatment (i.e. normal cells surrounding tumors over-expressing a receptor of interest). In addition, the finding that the addition of IL-6, a known transcriptional regulator of VEGF, had no effect on VEGF release is consistent with reports that the addition of exogenous IL-6 does not affect VEGF secretion.

TABLE 13
VEGF Release Assay

| | VEGF Release | | |
|---|---|---|---|
| Time Point | Control | BoNT/A | Noci-LH_{N}/A TVEMP |
| Day 1 | 100% | 69% | 119% |
| Day 2 | 100% | 57% | 123% |
| Day 3 | 100% | 53% | 125% |
| Day 4 | 100% | 57% | 104% |

Since VEGF is an inducer of migration, a compound that affects the release of VEGF should effect migration as well. Moreover, inhibition of exocytosis by a compound will also inhibit the release of additional factors involved in cell migration. To determine whether a botulinum toxin or TVEMP treatment could reduce or inhibit cell migration, a cell migration assay (Essen Bioscience, Ann Arbor, Mich.) was performed according to the manufacturer's instructions. On day 1, DU-145 cells were plated at 25,000 cells per well in a 96-well Essen ImageLock plate in growth media. On day 2 the cells were treated with either 10 nM BoNT/A, 40 nM Noci-LH_{N}/A TVEMP, or 90 nM Gal-LH_{N}/A TVEMP in growth media. As a positive control for inhibition of migration, cells were treated with 0.11 μM, 0.33 μM, or 1 μM Cytochalasin-D. As a negative control, cells were treated with media alone. On day 3, after the cells had reached 100% confluence, the cells were washed with media and then a 96-pin WoundMaker (Essen Bioscience, Ann Arbor, Mich.) was used to simultaneously create wounds in all the wells. After cell wounding, the media was removed and the cells were washed two times with 150 μL Dulbecco's Phosphate Buffered Saline with Ca²⁺ and Mg²⁺ and then 100 μL of media was added. The plate was then placed in an INCUCYTE™ scanner (Essen Bioscience, Ann Arbor, Mich.) and images were taken every 1 hour for 45 consecutive hours. The data was analyzed as relative wound density versus time using the INCUCYTE™ Cell Migration software. Relative wound density is designed to be zero at time zero, and 100% when the cell density inside the wound is the same as the cell density outside the initial wound.

The results are presented in Table 14. The results showed that cells pre-treated with either Noci-LH_{N}/A TVEMP or Gal-LH_{N}/A TVEMP migrated slightly slower than cells treated with media alone. The result showed that treatment with Noci-LH_{N}/A TVEMP or Gal-LH_{N}/A TVEMP resulted in a significant reduction in cell migration after 24 hours, about 10% reduction when compared to cells treated with media alone. Cells treated with BoNT/A did not exhibit an affect on cell migration. The cells treated with Cytochalasin-D did not migrate. When the same experiment was performed with PC-3 cells, that do not contain SNAP-25, rather than a reduction, an increase in migration was observed (data not shown), suggesting that initially, likely via activation of their ligand receptors, BoNT/A, Noci-LH_{N}/A TVEMP, and Gal-LH_{N}/A TVEMP function to increase migration. But after cleavage of SNAP-25 migration is reduced. As such, a longer exposure to a botulinum toxin and/or TVEMP will most likely result in more dramatic reduction in migration of such treated cells.

TABLE 14
Cell Migration Assay

Relative Wound Density at 24 Hours

| Treatment | Mean | Percent Relative to Media |
|---|---|---|
| Media Control | 78.2 ± 2.4 | 100% |
| BoNT/A | 78.6 ± 1.1 | 101% |
| Noci-LH_{N}/A TVEMP | 71.5 ± 3.3 | 91% |
| Gal-LH_{N}/A TVEMP | 69.5 ± 4.4 | 89% |
| Cytochalasin-D | 3.3 ± 0.2 | 4% |

Angiogenesis involves multiple steps; to achieve new blood vessel formation, endothelial cells must first escape their stable location by breaking through the basement membrane. Once this is achieved, endothelial cells migrate towards an angiogenic stimulus that might be released from cancer cells, or wound-associated macrophages. In addition, endothelial cells proliferate to provide the necessary number of cells for making a new vessel. Subsequent to this proliferation, the new outgrowth of endothelial cells needs to reorganize into a three-dimensionally tubular structure. To determine whether a botulinum toxin or TVEMP treatment could reduce or inhibit blood vessel formation, an in vitro Endothelial Tube Formation assay (Cell Biolabs, Inc., San Diego, Calif.) was performed according to the manufacturer's instructions. Human Umbilical Vein Endothelial Cells (HUVECs) were grown to 80% confluence in T-75 culture flasks until confluent. Cells were harvested and then plated at 500,000 cells per well for HUVECs in a 6-well plate for 24 hours. After incubation, cells were either kept untreated or treated with 2 nM or 5 nM of BoNT/A or 6 nM or 25 nM of Noci-LH_{N}/A TVEMP for 24 hours. As a positive control for inhibition, cells were treated with a collagenase inhibitor. As a negative control for inhibition, cells were treated with media alone. The cells were then harvested again and plated at 35,000 cells per well onto the ECM gel prepared from murine Engelbreth-Holm-Swan (EHS) tumor cells, which contain multiple angiogenic stimulating factors, such as, e.g., laminin, type IV collagen, heparan sulfate proteoglycans, entactin and growth factors such as FGF2 and TGF-βs. The cells were incubated for 3-4 hours on the ECM gels and then inspected under a microscope and photographed, either before or after staining with Calcein AM.

An Endothelial Tube Formation assay was also modified to use cells from a tumor cell line since it is known that tumor cells can form tubular structures to allow blood to flow into tumors. In this modified assay, cells from LNCaP, PC-3, DU-145, T24, and J82 cell lines were grown to 80% confluence in T-75 culture flasks. Cells were then harvested and plated at 400,000 cell per well in a 6-well plate containing 3 mL of an appropriate medium (Table 10), but with 1% serum. Cells were incubated in a 37° C. incubator under 5% carbon dioxide for 3 days. After incubation, cells were either kept untreated or treated with 20 nM of BoNT/A or 40 nM of Noci-LH_{N}/A TVEMP for 24 hours. The cells were then harvested, plated on ECM gel plates and inspected as described above.

The results show that in HUVEC, DU145 and J82 cells, and to a lesser degree in T24 and LNCaP cells, tubes formed on ECM plates treated with media alone, whereas treatment with a collagenase inhibitor prevented the formation of tubes (Table 15). No tubes formed in PC-3 cells. BoNT/A and Noci-LH_{N}/A TVEMP treatment of cells from a LNCaP prostate carcinoma cell line and a J82 bladder carcinoma cell line inhibited the formation of tubes. BoNT/A and Noci-LH_{N}/A TVEMP treatment had no effect on tube formation from HUVEC cultures. This inhibition of tube formation maybe due to inhibition of migration, delivery of receptors and other proteins to the membrane (motility factors and their receptors), adhesion molecules that interact with the matrix or other cells, and/or secretion of proteases.

TABLE 15
Endothelial Tube Formation Assay

| | Inhibition of Endothelial Tube Formation | | | |
|---|---|---|---|---|
| Cell Line | Media | Collagenase Inhibitor | BoNT/A | Noci-LH_{N}/A |
| LNCaP | No | Yes | Yes | Yes |
| PC-3 | — | — | — | — |
| DU-145 | No | ND | ND | ND |
| T24 | No | ND | ND | ND |
| J82 | No | Yes | Yes | Yes |
| HUVEC | No | ND | No | No |

ND, not determined

To conduct a human angiogenesis protein array screen, cells from a DU-145 prostate cancer cell line were plated in a 100 mm² plate containing Eagle's Minimum Essential Medium with 1% charcoal stripped FBS, 100 U/mL Penicillin, and 100 μg/mL Streptomycin. Cells were grown to a density of 5×10⁶ cells by incubating in a 37° C. incubator under 5% carbon dioxide overnight. After this incubation, the cells were washed by aspirating the media and rinsing the plate with 10 mL of 1×PBS. The washed cells were treated by replacing with fresh media containing 50 nM BoNT/A. For comparison, cells treated with media alone were run in parallel. After 24 hour treatment, the cells were washed, and harvested by lysing in freshly prepared Lysis Buffer (50 mM HEPES, 150 mM NaCl, 1.5 mM MgCl₂, 1 mM EGTA, 1%, 4-octylphenol polyethoxylate) on ice for 30 minutes with constant gentle agitation. Lysed cells were centrifuged at 14,000 g for 5 minutes at 4° C. to eliminate debris. The protein concentrations of cell lysates were measured by Bradford assay. To perform an assay, an array was incubated with 250 μL of each cell lysate containing 500 pg of protein. Array images were captured by scanning the blots with a Typhoon 9410 Imager and quantitation of array was performed with Image Quant TL V2005. Fold increased was determined by dividing signal from untreated over treated sample.

The results show that the majority of the 35 angiogenesis-related proteins detected were up-regulated in the cells treated with BoNT/A, compared to the untreated control (Table 16). Proteins that increased in expression were involved in promoting angiogenesis except for two proteins that are anti-angiogenic (endostatin and angiostatin). There was increased presence of GDNF, PDGF-AA, and FGF1 that promote cell proliferation, differentiation, cell growth and development. Proteins that promote or initiate angiogenesis were; Coagulation Factor III, EG-VEGF, Angiopoetin-1, Angiopoetin-2, and PD-ECGF. Expressions in proteins involved in glucose metabolism were; DPPIV, IGFBP-1, IGFBP-2, and IGFBP-3. Proteins that enhance cell-cell adhesion were also up-regulated; MIP-1, MMP-9, Endothelin-1, Platelet Factor 4 and TGF-61. The most significant increase was observed for Endocrine gland-derived vascular endothelial growth factor (EG-VEGF), which was almost 100-fold increased. The increase of these proteins in cell lysates may reflect their accumulation in the cytoplasm since exocytosis has been inhibited and the cells cannot release them to the media.

TABLE 16
Human Angiogenesis Array in DU145 Cell line

| | Mean Pixels Density | | Fold | |
|---|---|---|---|---|
| Analyte | Untreated | Treated | Increased | Function |
| External Control | 65451 | 68877 | 1.1 | — |
| Internal Control | 50052 | 59543 | 1.2 | — |
| Coagulation Factor III/TF | 12736 | 26726 | 2.1 | Promotes angiogenesis |
| GDNF | 156 | 428 | 2.7 | Promotes survival and differentiation |
| MIP-1 alpha | 153 | 535 | 3.5 | Chemotaxis |
| CXCL 16 | 3465 | 2352 | 0.7 | Cytokine |
| GM-CSF | 5001 | 1457 | 0.3 | Cytokine |
| Serpin E1 | 677 | 2214 | 3.3 | Inhibit proteases |
| Activin A | 552 | 1672 | 3.0 | Regulate morphogenesis in prostate |
| DPPIV | 3790 | 8923 | 2.4 | Glucose metabolism |
| HB-EGF | 8990 | 6717 | 0.7 | Cell proliferation |
| MMP-9 | 2454 | 5050 | 2.1 | Breakdown extracellular matrix |
| Serpin F1 | 743 | 882 | 1.2 | Inhibit proteases |
| TIMP-1 | 95918 | 86280 | 0.9 | Anti-angiogenic |
| Angiogenin | 6022 | 5468 | 0.9 | Promotes angiogenesis |
| EG-VEGF | 15 | 1368 | 88.3 | Promotes angiogenesis |
| IGFBP-1 | 122 | 1147 | 9.4 | Insulin growth factor protein |
| Pentraxin 3 | 119 | 732 | 6.2 | Involved in complement-mediated |
| | | | | clearance of apoptotic cells |
| TIMP-4 | 152 | 845 | 5.6 | Matrix metalloproteinases inhibitor |
| Angiopoietin-1 | 137 | 807 | 5.9 | Promotes angiogenesis |
| IGFBP-2 | 2379 | 8330 | 3.5 | Insulin growth factor protein |
| PD-ECGF | 942 | 12924 | 13.7 | Promotes angiogenesis |
| Thrombospondin-1 | 2138 | 12359 | 5.8 | Anti-angiogenic |
| Angiopoietin-2 | 129 | 1985 | 15.3 | Antagonist of angiopoietin 1 |
| Endostatin/Collagen XVIII | 2388 | 6800 | 2.8 | Anti-angiogenic |
| IGFBP-3 | 1145 | 11329 | 9.9 | Insulin like promotes cell survivor |
| PDGF-AA | 202 | 908 | 4.5 | Regulates cell proliferation, cellular |
| | | | | differentiation, cell growth, development |
| Angiostatin/Plasminogen | 142 | 893 | 6.3 | Anti-angiogenic |
| Endothelin-1 | 581 | 5828 | 10.0 | Vascular homeostasis |
| uPA | 30656 | 57108 | 1.9 | Serine protease |
| Amphiregulin | 33908 | 20736 | 0.6 | Interacts with the EGF/TGF-alpha |
| | | | | receptor to promote the growth |
| FGF1 | 1189 | 1875 | 1.6 | Promotes proliferation & differentiation |
| IL-8 | 45837 | 19261 | 0.4 | Angiogenic factor |
| FGF2 | 28018 | 23513 | 0.8 | Promotes proliferation & differentiation |
| LAP/TGF-β1 | 360 | 1914 | 5.3 | Increases extracellular matrix |
| | | | | production |
| Platelet Factor 4 | 456 | 819 | 1.8 | Cytokine |
| VEGF | 33513 | 31434 | 0.9 | Affects permeability |

Taken together, the experiments described in this Example show an overall decrease in angiogenic potential after treatment with botulinum toxin of TVEMP together with an observed increase in intracellular angiogenic proteins. This could be due to either activation of receptors for botulinum toxin or TVEMP that promotes angiogenesis and/or accumulation of vesicular proteins due to blockage of exocytosis after cleavage of SNARE proteins.

### Example 4

### Effects of Light Chain Delivery on Apoptosis

This example illustrates that treatment with a botulinum toxin or TVEMP will affect apoptosis to a degree sufficient to provide a therapeutic benefit to an individual suffering from a disease or disorder associated with aberrant new blood vessel formation.

The blockade of exocytosis resulting from a treatment with botulinum toxin or TVEMP based on LHN/A-G will likely result in decreased metabolic activity and decreased cell viability. As such, cancer cells with inhibited exocytosis capability due to a toxin or TVEMP effect will have a reduced ability to survive. To test whether such a treatment causes decreased cancer cell viability, three different assays were performed: a cell viability and metabolism assay, a Caspase-⅜ activity assay, and a human apoptotic protein array assay.

To determine whether a botulinum toxin or TVEMP treatment could decrease cancer cell viability, a CELLTITER 96® AQueous One Solution Cell Proliferation Assay cell metabolic activity assay (Promega Corp., Madison, Wis.) was performed according to the manufacturer's instructions. This assay is a colorimetric assay containing a tetrazolium compound [3-(4,5-dimethylthiazol-2-yl)-5-(3-carboxymethoxyphenyl)-2-(4-sulfophenyl)-2H-tetrazolium, inner salt; MTS] that is reduced by NADPH or NADH in metabolically active cells. The reduced MTS is a colored formazan product that can be measured at an absorbance of 490 nm. An appropriate density of cells from the cell lines MCF-7, SiMa, PC-12, 266.6, RWPE-1, and N2a, were plated into the wells of 96-well tissue culture plates containing 100 μL of an appropriate medium (Table 7), but without serum, and with or without 25 μg/mL of GT1b (Alexis Biochemicals, San Diego, Calif.). Cells were plated and incubated in a 37° C. incubator under 5% carbon dioxide until the cells differentiated, as assessed by standard and routine morphological criteria, such as growth arrest (approximately 3 days). The media was aspirated from each well and the differentiated cells were treated by replacing with fresh media containing 0 (untreated sample), 0.3125 nM, 1.25 nM, and 20 nM of a BoNT/A. After 24 hrs treatment, the cells were washed by aspirating the media and rinsing each well with 100 μL of 1×PBS. After washing, 100 μL of MTS solution was added to each well, incubated for 2 hours, and then the absorbance at 490 nm recorded with a 96-well plate reader. The quantity of formazan product as measured by the amount of 490 nm absorbance is directly proportional to the number of living cells in culture. A similar design can be employed to examine the effects of a TVEMP on cell viability.

The results show that a BoNT/A treatment decreased the metabolic activity in the cancerous cell lines tested (Table 17).

TABLE 17
Cell Metabolic Activity Assay

BoNT/A Concentration

| Cell Line | 0 nM | 0.3125 nM | 1.25 nM | 20 nM |
|---|---|---|---|---|
| MCF-7 | 1.60 | 1.45 | 1.41 | 1.30 |
| SiMa | 1.68 | 1.40 | 1.07 | 0.33 |
| PC-12 | 1.68 | 1.66 | 1.45 | 1.15 |
| 266.6 | 1.10 | 1.05 | 1.02 | 0.82 |
| RWPE-1 | 0.99 | 1.01 | 0.89 | 0.67 |
| N2a | 1.63 | 1.50 | 1.43 | 1.28 |

To further demonstrate that a botulinum toxin or TVEMP treatment could decrease cancer cell viability, a CELLTITER GLO® Luminescent Cell Viability Assay (Promega Corp., Madison, Wis.) was performed according to the manufacturer's instructions. In this assay, cell viability is quantified on the bases of the presence of ATP, which signals the presence of metabolically active cells. A decreased in ATP content corresponds to less metabolically active cells. Cells from the cell lines LNCaP, J82, T24, and DU-145 were differentiated as described above. The media was aspirated from each well and the differentiated cells were treated by replacing with fresh media containing either 1) 0 (untreated sample), 25 nM, and 50 nM of a BoNT/A; or 2) 0 (untreated sample), 250 nM, and 500 nM of a Noci-LH_{N}/A TVEMP. After 24 hrs treatment, the cells were washed by aspirating the media and rinsing each well with 100 μL of 1×PBS. After washing, 100 μL of CELLTITER GLO® reagent was added to each well. After ten minutes incubation at room temperature, the sample luminescence was measured using a SpectraMAX L luminescence reader (Molecular Devices, Sunnyvale, Calif.). Assays were performed in triplicate and cell viability was noted every day for four or five days.

The data shows that decreased viability was observed in cells from both a DU-145 prostate carcinoma cell line and a J82 bladder carcinoma cell line after BoNT/A treatments (Table 18) or Noci-LH_{N}/A TVEMP treatments (Table 19).

TABLE 18
Cell Viability Assay for BoNT/A

| | BoNT/A Concentration | | | | | | | |
|---|---|---|---|---|---|---|---|---|
| | DU-145 | | | | J82 | | | |
| Time | 0 nM | 25 nM | 0 nM | 50 nM | 0 nM | 25 nM | 0 nM | 50 nM |
| Day 1 | 3356 | 3291 | 404219 | 301228 | 3077 | 2853 | 543436 | 318900 |
| | | (0.385) | | (0.325) | | (0.223) | | (0.398) |
| Day 2 | 2360 | 2433 | 649139 | 394645 | 5211 | 4646 | 741025 | 493817 |
| | | (0.433) | | (0.174) | | (0.016) | | (0.129) |
| Day 4 | ND | ND | 1277552 | 809182 | ND | ND | 1242627 | 649797 |
| | | | | (0.058) | | | | (0.010) |
| Day 5 | 4823 | 2325 | ND | ND | 7384 | 4262 | ND | ND |
| | | (0.0001) | | | | (0.0001) | | |

P value indicating significant difference relative to non-treated control is listed in parenthesis.
ND, not determined

TABLE 19
Cell Viability Assay for Noci-LH_{N}/A TVEMP

| | Noci-LH_{N}/A TVEMP Concentration | | | | | | | |
|---|---|---|---|---|---|---|---|---|
| | DU-145 | | | | J82 | | | |
| Time | 0 nM | 250 nM | 0 nM | 500 nM | 0 nM | 250 nM | 0 nM | 500 nM |
| Day 1 | 3356 | 3630 | 404219 | 408023 | 3077 | 3189 | 543436 | 406420 |
| | | (0.087) | | (0.959) | | (0.223) | | (0.103) |
| Day 2 | 2360 | 2379 | 649139 | 622596 | 5211 | 4639 | 741025 | 677236 |
| | | (0.876) | | (0.802) | | (0.015) | | (0.581) |
| Day 4 | | | 1277552 | 1030346 | | | 1242627 | 854124 |
| | | | | (0.171) | | | | (0.020) |
| Day 5 | 4823 | 3595 | | | 7384 | 6349 | | |
| | | (0.0003) | | | | (0.009) | | |

P value indicating significant difference relative to non-treated control is listed in parenthesis.
ND, not determined

To determine whether a botulinum toxin or TVEMP treatment decreased cancer cell viability by an apoptotic process, the activity of Caspase-⅜ was measured in cell treated with BoNT/A. Cells from the cell lines LNCaP, J82, and T24 were differentiated as described above. The media was aspirated from each well and the differentiated cells were treated by replacing with fresh media containing either 1) 0 (untreated sample), 0.5 nM, 5 nM, and 50 nM of a BoNT/A; or 2) 0 (untreated sample), 1.6 nM, 16 nM, and 166 nM of a Noci-LH_{N}/A TVEMP. After 24 hrs treatment, the cells were washed by aspirating the media and rinsing each well with 100 μL of 1×PBS To measure cellular caspase 9 activity, 50 μL of CASPASE-GLO® 9 (Promega, Corp., Madison, Wis.) reagent was added to the culture media of each well. After 30 minute incubation at 37° C., the luminescence of each sample was measured using a Spectramax L luminometer (Molecular Devices, Sunnyvale, Calif.). T24 does not express SNAP-25 and should not be sensitive to treatment with BoNT/A or Noci-LH_{N}/A TVEMP.

The data shows that an effect on Caspase ⅜ activity was most prevalent in LNCaP cell after exposure to BoNT/A, indicating that LNCaP cell line viability decreases with BoNT/A treatment (Table 20). These data are supported by the cell viability assays measuring the number of live and dead cells in populations treated with BoNT/A (Table 18). Although cells from a J82 cell line did not show significant differences in Caspase ⅜ activity, this cell line did contain a higher amount of dead cells after BoNT/A or Noci-LH_{N}/A TVEMP treatments (Table 19). The reason for the observation of no caspase activity in J82 cells could be due to at least two possibilities: 1) the timing of BoNT/A treatment to detect Caspase ⅜ activity is different for J82 and LNCaP (e.g., Caspase ⅜ activation may had occur earlier in J82 cells); or 2) the cell death pathway for J82 is independent of Caspase ⅜.

TABLE 20
Caspase 3/8 Activity Assay

| | BoNT/A Concentration | | | | Noci-LH_{N}/A TVEMP | | | |
|---|---|---|---|---|---|---|---|---|
| Cell | | | | 50 | | | | 166 |
| Line | 0 nM | 0.5 nM | 5 nM | nM | 0 nM | 1.6 nM | 16 nM | nM |
| LNCaP | 270 | 283 | 239 | 572 | 218 | 232 | 233 | 263 |
| T24 | 656 | 612 | 634 | 646 | 637 | 602 | 623 | 617 |
| J82 | 235 | 146 | 256 | 194 | 132 | 133 | 103 | 98 |

To test whether cell death of cells treated with a botulinum toxin or TVEMP was directed by a process independent of Caspase ⅜ pathway, cells were assayed for the presence of cleaved nuclear poly (ADP-ribose) polymerase (PARP). PARP is a 116 kDa nuclear poly (ADP-ribose) polymerase and appears to be involved in DNA repair in response to environmental stress. This protein can be cleaved by many ICE-like caspases in vitro and is one of the main cleavage targets of Caspase-3 in vivo. In human PARP, the cleavage occurs between Asp214 and Gly215, which separates the PARP amino-terminal DNA binding domain (24 kDa) from the carboxy-terminal catalytic domain (89 kDa). PARP helps cells to maintain their viability; cleavage of PARP facilitates cellular disassembly and serves as a marker of cells undergoing apoptosis. To determine whether changes in cell viability are due to cells undergoing apoptosis, cells from the cell lines DU-145 and J82 were differentiated as described above. The media was aspirated from each well and the differentiated cells were treated by replacing with fresh media containing either 1) 0 (untreated sample) and 50 nM of a BoNT/A; or 2) 0 (untreated sample) and 500 nM of a Noci-LH_{N}/A TVEMP. After 48 hrs treatment, the cells were washed, harvested and Western blot analysis performed as described in Example 1, except an α-PARP antibodies were used as the primary antibody. Cells from both cell lines showed an increased of cleaved PARP after 2 days of Noci-LH_{N}/A TVEMP treatment. However, the presence of cleaved PARP was minimal in cells from both cell lines treated with a BoNT/A.

To conduct a human apoptosis protein array screen, cells from a DU-145 prostate cancer cell line were treated with a BoNT/A, harvested, and assayed as described above in Example 3. The results show that after treatment of cells from the DU-145 cell line with 50 nM BonT/A for 24 hours, most of apoptosis-related proteins remained unchanged when compared to control. There were only 10 apoptotic-related proteins where expression decreased from 1.5-fold to 2.4-fold (Table 21). A decreased in expression was noted in three anti-apoptotic proteins (Livin, survivin, and BCL-x), two cell cycle related proteins (Claspin and P27), antioxidant related protein (PON2), chaperone protein (clusterin) and two pro-apoptotic related proteins (Bax and Cytochrome C).

TABLE 21
Human Apoptosis Array in DU-145 Cell line

| | Mean Pixel density | | | |
|---|---|---|---|---|
| Analyte | Untreated | Treated | Fold Decrease | Function |
| Livin | 644.1 | 469.7 | 1.7 | Anti-apoptotic |
| Cytochrome c | 3423 | 1889 | 1.9 | Pro-apoptotic |
| XIAP | 10099 | 10045 | 1.0 | Anti-apoptotic |
| HTRA2/Omi | 7542 | 9368 | 0.8 | IAP antagonist |
| Clusterin | 1139 | 816 | 1.6 | Chaperones misfolded proteins |
| TNF rRI/TNFRSF1A | 2036 | 1467 | 1.5 | Activates NFkB |
| HSP70 | 7058 | 9669 | 0.7 | Stress response chaperone |
| Claspin | 6630 | 3390 | 2.0 | Cell cycle check point |
| Survivin | 8717 | 3739 | 2.4 | Anti-apoptotic |
| HSP60 | 945 | 855 | 1.2 | Stress response chaperone |
| cIAP-2 | 2862 | 3156 | 0.9 | Inhibitor of Apoptosis (IAP) |
| SMAC/Diablo | 8379 | 7132 | 1.2 | Promotes caspase activation by interaction |
| | | | | with IAP proteins |
| HSP27 | 5716 | 5683 | 1.0 | Stress response chaperone |
| cIAP-1 | 16916 | 15297 | 1.1 | Inhibitor of Apoptosis (IAP) |
| Phospho-Rad17 | 1646 | 999 | 1.8 | cell cycle check point |
| HO-2/HMOX2 | 8930 | 8934 | 1.0 | Microsomal enzyme |
| Catalase | 18742 | 18710 | 1.0 | Prevent cell damage from oxidative stress |
| p53 | 19134 | 22007 | 0.9 | Induces apoptosis |
| HO-1/HMOX1/HSP32 | 9878 | 11333 | 0.9 | Microsomal enzyme |
| Cleaved Caspase-3 | 715 | 614 | 1.3 | Downstream mediator |
| | | | | of apoptotis |
| p53 | 8623 | 11225 | 0.8 | Induces apoptosis |
| HIF-1 alpha | 6832 | 6703 | 1.0 | Binds to hypoxia response elements |
| Pro-Caspase-3 | 36318 | 42668 | 0.9 | Downstream mediator |
| | | | | of apoptotis |
| p53 | 20019 | 24725 | 0.8 | Induces apoptosis |
| Fas/TNFSF6 | 34978 | 35878 | 1.0 | Induces apoptosis |
| Bcl-x | 571 | 445 | 1.6 | Anti-apoptotic |
| p27 | 1293 | 852 | 1.7 | Cell cycle check point |
| FADD | 9996 | 8647 | 1.2 | Induces apoptosis |
| Bcl-2 | 967 | 1427 | 0.7 | Anti-apoptotic |
| p21 | 1062 | 1029 | 1.1 | Blocks cell cycle |
| TRAIL R2/DR5 | 25985 | 21477 | 1.2 | Induces apoptosis |
| Bax | 2097 | 1436 | 1.6 | Apoptotic activator |
| PON2 | 2611 | 1784 | 1.5 | Antioxidant enzyme |
| TRAIL R1 | 28443 | 20518 | 1.4 | Induces apoptosis |
| Bad | 5097 | 5932 | 0.9 | Pro-apoptotic |

Taken together, the experiments described in this Example show that treatment with a BoNT/A or TVEMP results in decreased metabolic activity and decreased cells viability. Events related to apoptosis were identified following light chain delivery into cancer cells, Caspase ⅜ activity was observed after treatment with BoNT/A in LNCaP cells as well as increased cleavage of PARP, the main substrate for Caspase 3 was observed after treatment with Noci-LH_{N}/A TVEMP in the DU-145 and J82 cells, showing that cells are pushed towards apoptosis after treatment with a BoNT/A or a TVEMP. Overall, the amounts of proteins involved with apoptosis in the cell lysates did not change after treatment with BoNT/A. Most of the pro-apoptotic and anti-apoptotic proteins exert their function by translocating from the cytoplasm to the mitochondria without changes in total protein amount. The small changes detected may be a short term response of the tumor cells to the inhibition of exocytosis and the interference with the input from the autocrine or paracrine loops that the cancer cell needs to survive. Eventually these cells will be pushed into apoptosis due to the lack of survival signals.

### Example 5

### Treatment of Cancer Associated with Aberrant New Blood Vessel Formation

The following examples are provided by way of describing specific embodiments without intending to limit the scope of the invention in any way.

A physician examines a patient who complains of a lump in her left breast and diagnoses her with breast cancer. The patient is treated by local administration a composition comprising a TVEMP as disclosed herein in the vicinity of the affected area. The patient's condition is monitored and after about 1-7 days after treatment, the physician notes that the growth of the malignant tumor has slowed down. At one and three month check-ups, the physician determines that there is a decrease in the blood vessel architecture associated with the tumor as well as a decrease in the size of the tumor. This reduction in tumor size and/or aberrant new blood vessel formation indicate successful treatment with the composition comprising a TVEMP. In addition, a systemic administration of a composition comprising a TVEMP as disclosed herein could also be used to administer a disclosed TVEMP to treat the breast cancer.

A physician examines a patient who complains of difficulty in urinating and diagnoses him with prostate cancer. The patient is treated systemically by intravenous administration a composition comprising a TVEMP as disclosed herein. The patient's condition is monitored and after about 1-7 days after treatment, the physician determines that the size of the prostate has become smaller. At one and three month check-ups, the physician determines that there is a decrease in the blood vessel architecture associated with the tumor as well as a decrease in the size of the tumor and that serum PSA levels are within the normal range. This reduction in tumor size and/or reduces serum PSA levels indicates successful treatment with the composition comprising a TVEMP. In addition, a local administration of a composition comprising a TVEMP as disclosed herein could also be used to administer a disclosed TVEMP to treat the prostate cancer.

A physician examines a patient who complains of wheezing when he breathes and diagnoses him with lung cancer. The patient is treated systemically by intravenous administration a composition comprising a TVEMP as disclosed herein. The patient's condition is monitored and after about 1-7 days after treatment, the physician notes that the growth of the malignant tumor has slowed down. At one and three month check-ups, the patient indicates that his breathing is normal and the physician determines that there is a decrease in the blood vessel architecture associated with the tumor as well as a decrease in the size of the tumor. The normal breathing, reduction in tumor size and/or aberrant new blood vessel formation indicate successful treatment with the composition comprising a TVEMP. In addition, systemic administration could also be used to administer a disclosed TVEMP to treat cancer. In addition, administration by inhalation could also be used to administer a disclosed TVEMP to treat the lung cancer.

A physician examines a patient who complains of pelvic pain and diagnoses her with bladder cancer. The patient is treated by local administration a composition comprising a TVEMP as disclosed herein in the vicinity of the affected area. The patient's condition is monitored and after about 1-7 days after treatment, the physician notes that the growth of the malignant tumor has slowed down. At one and three month check-ups, the patient indicates that the pelvic pain is gone and the physician determines that there is a decrease in the blood vessel architecture associated with the tumor as well as a decrease in the size of the tumor. The reduction in pain, tumor size and/or aberrant new blood vessel formation indicate successful treatment with the composition comprising a TVEMP. In addition, a systemic administration of a composition comprising a TVEMP as disclosed herein could also be used to administer a disclosed TVEMP to treat the bladder cancer.

A physician examines a patient who complains of abdominal pain and diagnoses her with colon cancer. The patient is treated by systemically by intravenous administration of a composition comprising a TVEMP as disclosed herein. The patient's condition is monitored and after about 1-7 days after treatment, and the physician notes that the growth of the malignant tumor has slowed down. At one and three month check-ups, the patient indicates that the abdominal pain is gone and the physician determines that there is a decrease in the blood vessel architecture associated with the tumor as well as a decrease in the size of the tumor. The reduced pain, tumor size and/or aberrant new blood vessel formation indicate successful treatment with the composition comprising a TVEMP. In addition, a local administration of a composition comprising a TVEMP as disclosed herein could also be used to administer a disclosed TVEMP to treat the colon cancer.

A physician examines a patient who complains of headaches and dizziness and diagnoses him with a neuroblastoma. The patient is treated by intracranial administration a composition comprising a TVEMP as disclosed herein in the vicinity of the affected area. The patient's condition is monitored and after about 1-7 days after treatment, the physician determines that there is a decrease in the blood vessel architecture associated with the tumor as well as a decrease in the size of the tumor. At one and three month check-ups, the patient indicates that he no longer suffers from headaches and dizziness and the physician determines that there is a decrease in the blood vessel architecture associated with the tumor as well as a decrease in the size of the tumor. The disappearance of headache, dizziness and/or the reduction in tumor size and/or aberrant new blood vessel formation indicate successful treatment with the composition comprising a TVEMP.

### Example 6

Treatment of Disease or Disorder Associated with Aberrant New Blood Vessel Formation

The following examples are provided by way of describing specific embodiments without intending to limit the scope of the invention in any way.

A physician examines a patient who complains of poor vision and diagnoses her with age-related macular degeneration. The patient is treated by local administration a composition comprising a TVEMP as disclosed herein in the vicinity of the affected area. The patient's condition is monitored and after about 1-7 days after treatment, the physician notes that the growth of new blood vessels has slowed down. At one and three month check-ups, the physician determines that the architecture of the blood vessels in the retina appears normal and the patient's vision improves. This reduction in aberrant new blood vessel formation indicates successful treatment with the composition comprising a TVEMP. A similar approach can be used to treat an individual suffering from a retinopathy, a different macula degeneration, or a choroidal neovascularization.

A physician examines a patient who complains of chest pains and diagnoses him with arteriosclerosis and high blood pressure. The patient is treated by local administration a composition comprising a TVEMP as disclosed herein in the vicinity of the affected area. The patient's condition is monitored and after about 1-7 days after treatment, the physician notes that the patient's blood pressure has dropped. At one and three month check-ups, the physician determines that there is a decrease in vessel blockage, a further lowering of the patient's blood pressure, and that the frequency of chest pains is lower. This reduction in vessel blockage, blood pressure and chest pain indicates successful treatment with the composition comprising a TVEMP. In addition, a systemic administration of a composition comprising a TVEMP as disclosed herein could also be used to administer a disclosed TVEMP to treat arteriosclerosis and high blood pressure.

A physician examines a patient who complains of painful skin itching and diagnoses her with psoriasis. The patient is treated by topical administration of a composition comprising a TVEMP as disclosed herein. The patient's condition is monitored and after about 1-7 days after treatment, the physician determines that the extent of psoriasis is reduced slightly. At one and three month check-ups, the patient indicates that he no longer suffers any itching and the physician determines that the psoriasis is gone. The reduction in itching and disappearance of the psoriasis indicates successful treatment with the composition comprising a TVEMP. In addition, a systemic or injection-based administration of a composition comprising a TVEMP as disclosed herein could also be used to administer a disclosed TVEMP to treat the psoriasis.

A physician examines a patient who complains of wheezing when she breathes and diagnoses her with idiopathic pulmonary fibrosis. The patient is treated systemically by intravenous administration a composition comprising a TVEMP as disclosed herein. The patient's condition is monitored and after about 1-7 days after treatment, the physician notes that the patient is not wheezing as often. At one and three month check-ups, the patient indicates that her breathing is normal and the physician determines that there is a decrease in the blood vessel architecture associated with pulmonary fibrosis. The normal breathing and/or the reduction in aberrant new blood vessel formation indicate successful treatment with the composition comprising a TVEMP. In addition, systemic administration could also be used to administer a disclosed TVEMP to treat idiopathic pulmonary fibrosis. In addition, administration by inhalation could also be used to administer a disclosed TVEMP to treat the idiopathic pulmonary fibrosis.

A physician examines a patient who complains of pain in his joints and diagnoses him with a rheumatoid arthritis. The patient is treated by systemic administration of a composition comprising a TVEMP as disclosed herein. The patient's condition is monitored and after about 1-7 days after treatment, the physician determines that there is a decrease in the patient's joint pain. At one and three month check-ups, the patient indicates that he no longer feels joint pain and the physician determines that there is a decrease in the blood vessel architecture associated with joints that gave the patient pain. The disappearance of pain and/or reduction in aberrant new blood vessel formation indicate successful treatment with the composition comprising a TVEMP.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a disease or disorder associated with aberrant new blood vessel formation in a mammal, the method comprising the step of administering to the mammal in need thereof a therapeutically effective amount of a composition including a TVEMP comprising a targeting domain, a Clostridial toxin translocation domain and a Clostridial toxin enzymatic domain, and an exogenous protease cleavage site, wherein administration of the composition decreases a symptom of a disease or disorder associated with aberrant new blood vessel formation.

2. The method of claim 1, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

3. The method of claim 1, wherein the targeting domain is an apelin targeting domain, an anthrax protective antigen (APA) targeting domain, an angiogenic factor with G patch and FHA domain 1 (AGGF1) targeting domain, an angiopoietin targeting domain, an angiostatin targeting domain, an angiogenin targeting domain, an endothelin targeting domain, an endothelial cell-specific molecule (ECSM) targeting domain, an ephrin targeting domain, an erythropoietin targeting domain, a hepatocyte growth factor (HGF) targeting domain, a netrin targeting domain, a brain specific angiogenesis inhibitor (BSAI) targeting domain, a Delta-like (DII) targeting domain, a jagged targeting domain, a semaphorin (Sema) targeting domain, a thrombospondin targeting domain, a chondromodulin targeting domain, a 16 kDa prolactin fragment targeting domain, a granulocyte macrophage-colony stimulating factor (GM-CSF) targeting domain, an Interferon-α (IFN-α) targeting domain, a pigment epithelium-derived factor (PEDF) targeting domain, a troponin targeting domain, a Vasohibin (VASH) targeting domain, a Urocortin (Ucn) targeting domain, a C-motif cytokine ligand targeting domain, a C—C-motif cytokine ligand targeting domain, a C—X—C-motif cytokine ligand targeting domain, and an endothelial-monocyte activating polypeptide (EMAP) targeting domain.

4. The method of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

5. The method of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

6. The method of claim 1, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

7. The method of claim 1, wherein the disease or disorder associated with aberrant new blood vessel formation is a retinopathy, a macula degeneration, a choroidal neovascularization, an atherosclerosis, a coronary atherosclerotic plaque formation, an endometriosis, an idiopathic pulmonary fibrosis, chronic inflammatory/fibroproliferative disorder, a rheumatoid arthritis, a psoriasis, or a cancer.

8. A TVEMP comprising a targeting domain, a Clostridial toxin translocation domain and a Clostridial toxin enzymatic domain, and an exogenous protease cleavage site.

9. The TVEMP of claim 8, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

10. The TVEMP of claim 8, wherein the targeting domain is an apelin targeting domain, an anthrax protective antigen (APA) targeting domain, an angiogenic factor with G patch and FHA domain 1 (AGGF1) targeting domain, an angiopoietin targeting domain, an angiostatin targeting domain, an angiogenin targeting domain, an endothelin targeting domain, an endothelial cell-specific molecule (ECSM) targeting domain, an ephrin targeting domain, an erythropoietin targeting domain, a hepatocyte growth factor (HGF) targeting domain, a netrin targeting domain, a brain specific angiogenesis inhibitor (BSAI) targeting domain, a Delta-like (DII) targeting domain, a jagged targeting domain, a semaphorin (Sema) targeting domain, a thrombospondin targeting domain, a chondromodulin targeting domain, a 16 kDa prolactin fragment targeting domain, a granulocyte macrophage-colony stimulating factor (GM-CSF) targeting domain, an Interferon-α (IFN-α) targeting domain, a pigment epithelium-derived factor (PEDF) targeting domain, a troponin targeting domain, a Vasohibin (VASH) targeting domain, a Urocortin (Ucn) targeting domain, a C-motif cytokine ligand targeting domain, a C—C-motif cytokine ligand targeting domain, a C—X—C-motif cytokine ligand targeting domain, and an endothelial-monocyte activating polypeptide (EMAP) targeting domain.

11. The TVEMP of claim 8, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

12. The TVEMP of claim 8, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

13. The TVEMP of claim 8, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

14. A composition comprising a TVEMP of claim 8.

15. The composition of claim 14, wherein the composition is a pharmaceutical composition.
